SEQUENCE LISTING (175 sequences)

SEQ ID NO: 1 - protein, 117 aa, Homo sapiens
ALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 2 - protein, 72 aa, Homo sapiens
ITLEDVAVDFTWEEWQLLGAAQKDLYRDVMLENYSNLVAVGYQASKPDALFKLEQGEQLWTIEDGIHSGACS

SEQ ID NO: 3 - protein, 73 aa, Homo sapiens
VMFEEVSVCFTSEEWACLGPIQRALYWDVMLENYGNVTSLEWETMTENEEVTSKPSSSQRADSHKGTSKRLQG

SEQ ID NO: 4 - protein, 72 aa, Homo sapiens
VSFKDVAVDFTQEEWQQLDPDEKITYRDVMLENYSHLVSVGYDTTKPNVIIKLEQGEEPWIMGGEFPCQHSP

SEQ ID NO: 5 - protein, 74 aa, Homo sapiens
VKIEDMAVSLILEEWGCQNLARRNLSRDNRQENYGSAFPQGGENRNENEESTSKAETSEDSASRGETTGRSQKE

SEQ ID NO: 6 - protein, 72 aa, Homo sapiens
LTFKDVFVDFTLEEWQQLDSAQKNLYRDVMLENYSHLVSVGYLVAKPDVIFRLGPGEESWMADGGTPVRTCA

SEQ ID NO: 7 - protein, 72 aa, Homo sapiens
VTFEDVTLGFTPEEWGLLDLKQKSLYREVMLENYRNLVSVEHQLSKPDVVSQLEEAEDFWPVERGIPQDTIP

SEQ ID NO: 8 - protein, 322 aa, Homo sapiens
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 9 - protein, 322 aa, Homo sapiens
CHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 10 - protein, 478 aa, Homo sapiens
LRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 11 - protein, 386 aa, Homo sapiens
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 12 - protein, 1291 aa, Homo sapiens
MSSLPGCIGLDAATATVESEEIAELQQAVVEELGISMEELRHFIDEELEKMDCVQQRKKQLAELETWVIQKESEVAHVDQLFDDASRAVTNCESLVKDFYSKLGLQYRDSSSEDESSRPTEIIEIPDEDDDVLSIDSGDAGSRTPKDQKLREAMAALRKSAQDVQKFMDAVNKKSSSQDLHKGTLSQMSGELSKDGDLIVSMRILGKKRTKTWHKGTLIAIQTVGPGKKYKVKFDNKGKSLLSGNHIAYDYHPPADKLYVGSRVVAKYKDGNQVWLYAGIVAETPNVKNKLRFLIFFDDGYASYVTQSELYPICRPLKKTWEDIEDISCRDFIEEYVTAYPNRPMVLLKSGQLIKTEWEGTWWKSRVEEVDGSLVRILFLDDKRCEWIYRGSTRLEPMFSMKTSSASALEKKQGQLRTRPNMGAVRSKGPVVQYTQDLTGTGTQFKPVEPPQPTAPPAPPFPPAPPLSPQAGDSDLESQLAQSRKQVAKKSTSFRPGSVGSGHSSPTSPALSENVSGGKPGINQTYRSPLGSTASAPAPSALPAPPAPPVFHGMLERAPAEPSYRAPMEKLFYLPHVCSYTCLSRVRPMRNEQYRGKNPLLVPLLYDFRRMTARRRVNRKMGFHVIYKTPCGLCLRTMQEIERYLFETGCDFLFLEMFCLDPYVLVDRKFQPYKPFYYILDITYGKEDVPLSCVNEIDTTPPPQVAYSKERIPGKGVFINTGPEFLVGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKRIRAGTELTWDYNYEVGSVEGKELLCCCGAIECRGRLL

SEQ ID NO: 13 - protein, 565 aa, Homo sapiens
VGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKRIRAGTELTWDYNYEVGSVEGKELLCCCGAIECRGRLL

SEQ ID NO: 14 - protein, 826 aa, synthetic construct
  source 1..826: note = Transcription-activator like effector (TALE) domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVA

SEQ ID NO: 15 - protein, 826 aa, synthetic construct
  source 1..826: note = TALE domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVA

SEQ ID NO: 16 - protein, 831 aa, synthetic construct
  source 1..831: note = TALE domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 17 - DNA, 19 bp, synthetic construct
  source 1..19: mol_type = other DNA; note = Binding site
tctctcctaccctcccgct

SEQ ID NO: 18 - protein, 831 aa, synthetic construct
  source 1..831: note = TALE domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 19 - DNA, 19 bp, synthetic construct
  source 1..19: mol_type = other DNA; note = Binding site
tggtccttcctctcccgct

SEQ ID NO: 20 - protein, 831 aa, synthetic construct
  source 1..831: note = TALE domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 21 - DNA, 19 bp, synthetic construct
  source 1..19: mol_type = other DNA; note = Binding site
tcgctccgtgacttccctt

SEQ ID NO: 22 - protein, 1441 aa, synthetic construct
  source 1..1441: note = Artificial transcription repressor (ATR) sequence, catalytically inactive Cas9
MGGRRVRWEVYISRALWLTREPTAYWLIEINTTHYRETQATGATMYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVG

SEQ ID NO: 23 - DNA, 20 bp, synthetic construct
  source 1..20: mol_type = other DNA; note = Genomic target site for guide RNA (gRNA) #1 for beta2-microglobulin gene
tataagtggaggcgtcgcgc

SEQ ID NO: 24 - DNA, 20 bp, synthetic construct
  source 1..20: mol_type = other DNA; note = Genomic target site for gRNA #2 for beta2-microglobulin gene
gcccgaatgctgtcagcttc

SEQ ID NO: 25 - DNA, 20 bp, synthetic construct
  source 1..20: mol_type = other DNA; note = Genomic target site for gRNA #3 for beta2-microglobulin gene
tgcgtcgctggcttggagac

SEQ ID NO: 26 - DNA, 20 bp, synthetic construct
  source 1..20: mol_type = other DNA; note = Genomic target site for gRNA #4 for beta2-microglobulin gene
ccaatcaggacaaggcccgc

SEQ ID NO: 27 - DNA, 20 bp, synthetic construct
  source 1..20: mol_type = other DNA; note = Genomic target site for gRNA #5 for beta2-microglobulin gene
agggtaggagagactcacgc

SEQ ID NO: 28 - DNA, 20 bp, synthetic construct
  source 1..20: mol_type = other DNA; note = Genomic target site for gRNA #6 for beta2-microglobulin gene
gcgggccaccaaggagaact

SEQ ID NO: 29 - DNA, 20 bp, synthetic construct
  source 1..20: mol_type = other DNA; note = Genomic target site for gRNA #7 for beta2-microglobulin gene
gctactctctctttctggcc

SEQ ID NO: 30 - DNA, 20 bp, synthetic construct
  source 1..20: mol_type = other DNA; note = Genomic target site for gRNA #8 for beta2-microglobulin gene
ctcccgctctgcaccctctg

SEQ ID NO: 31 - DNA, 20 bp, synthetic construct
  source 1..20: mol_type = other DNA; note = Genomic target site for gRNA #9 for beta2-microglobulin gene
tttggcctacggcgacggga

SEQ ID NO: 32 - DNA, 20 bp, synthetic construct
  source 1..20: mol_type = other DNA; note = Genomic target site for gRNA #10 for beta2-microglobulin gene
ggggcaagtagcgcgcgtcc

SEQ ID NO: 33 - DNA, 20 bp, synthetic construct
  source 1..20: mol_type = other DNA; note = Genomic target site for gRNA #11 for beta2-microglobulin gene
tagtccagggctggatctcg

SEQ ID NO: 34 - DNA, 19 bp, synthetic construct
  source 1..19: mol_type = other DNA; note = Binding site
tacccagattggccccact

SEQ ID NO: 35 - DNA, 19 bp, synthetic construct
  source 1..19: mol_type = other DNA; note = Binding site
tacctagaggagaaaggtt

SEQ ID NO: 36 - protein, 846 aa, Homo sapiens
MVAELISEEDLEFMKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 37 - protein, 831 aa, synthetic construct
  source 1..831: note = TALE domain BCL11A #1
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALESIVAQLSRPDPALAALTNDHLVALACLGGRPALDAVKKGLPHAPALIKRTNRRIPERTSHRVAGSGGG

SEQ ID NO: 38 - DNA, 19 bp, synthetic construct
  source 1..19: mol_type = other DNA; note = BCL11A gene binding site
tccaaaagccagtctcacc

SEQ ID NO: 39 - protein, 831 aa, synthetic construct
  source 1..831: note = TALE domain BCL11A #2
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPALDAVKKGLPHAPALIKRTNRRIPERTSHRVAGSGGG

SEQ ID NO: 40 - DNA, 19 bp, synthetic construct
  source 1..19: mol_type = other DNA; note = BCL11A gene binding site
tctccccgggaatcgtttt

SEQ ID NO: 41 - protein, 831 aa, synthetic construct
  source 1..831: note = TALE domain BCL11A #3
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 42 - DNA, 19 bp, synthetic construct
  source 1..19: mol_type = other DNA; note = BCL11A gene binding site
tcctcccgctgcacacttg

SEQ ID NO: 43 - protein, 831 aa, synthetic construct
  source 1..831: note = TALE domain BCL11A #4
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALESIVAQLSRPDPALAALTNDHLVALACLGGRPALDAVKKGLPHAPALIKRTNRRIPERTSHRVAGSGGG

SEQ ID NO: 44 - DNA, 19 bp, synthetic construct
  source 1..19: mol_type = other DNA; note = BCL11A gene binding site
tagtcatccccacaatagt

SEQ ID NO: 45 - protein, 831 aa, synthetic construct
  source 1..831: note = TALE domain BCL11A #5
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 46 - DNA, 19 bp, synthetic construct
  source 1..19: mol_type = other DNA; note = BCL11A gene binding site
tcccgctgccttttgtgcc

SEQ ID NO: 47 - protein, 831 aa, synthetic construct
  source 1..831: note = TALE domain BCL11A #6
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 48 - DNA, 19 bp, synthetic construct
  source 1..19: mol_type = other DNA; note = BCL11A gene binding site
tcctcgcgcttgccctccc

SEQ ID NO: 49 - protein, 831 aa, synthetic construct
  source 1..831: note = TALE domain BCL11A #7
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALESIVAQLSRPDPALAALTNDHLVALACLGGRPALDAVKKGLPHAPALIKRTNRRIPERTSHRVAGSGGG

SEQ ID NO: 50 - DNA, 19 bp, synthetic construct
  source 1..19: mol_type = other DNA; note = BCL11A gene binding site
tcccccggccctagctcct

SEQ ID NO: 51 - protein, 831 aa, synthetic construct
  source 1..831: note = TALE domain BCL11A #8
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 52 - DNA, 19 bp, synthetic construct
  source 1..19: mol_type = other DNA; note = BCL11A gene binding site
tcctggtccgcccccagca

SEQ ID NO: 53 - protein, 831 aa, synthetic construct
  source 1..831: note = TALE domain BCL11A #9
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 54 - DNA, 19 bp, synthetic construct
  source 1..19: mol_type = other DNA; note = BCL11A gene binding site
tgccgagacctcttctcga

SEQ ID NO: 55 - protein, 831 aa, synthetic construct
  source 1..831: note = TALE domain BCL11A #10
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVKRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 56 - DNA, 19 bp, synthetic construct
  source 1..19: mol_type = other DNA; note = BCL11A gene binding site
tcggctttgcaaagcattt

SEQ ID NO: 57 - protein, 831 aa, synthetic construct
  source 1..831: note = TALE domain BCL11A #11
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 58 - DNA, 19 bp, synthetic construct
  source 1..19: mol_type = other DNA; note = BCL11A gene binding site
tgcaaagccgagtttcacc

SEQ ID NO: 59 - protein, 831 aa, synthetic construct
  source 1..831: note = TALE domain BCL11A #12
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNHGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALESIVAQLSRPDPALAALTNDHLVALACLGGRPALDAVKKGLPHAPALIKRTNRRIPERTSHRVAGSGGG

SEQ ID NO: 60 - DNA, 19 bp, synthetic construct
  source 1..19: mol_type = other DNA; note = BCL11A gene binding site
tacagttgccctgcaaaat

SEQ ID NO: 61 - protein, 831 aa, synthetic construct
  source 1..831: note = TALE domain BCL11A #13
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALESIVAQLSRPDPALAALTNDHLVALACLGGRPALDAVKKGLPHAPALIKRTNRRIPERTSHRVAGSGGG

SEQ ID NO: 62 - DNA, 19 bp, synthetic construct
  source 1..19: mol_type = other DNA; note = BCL11A gene binding site
tccgccctgggtactttct

SEQ ID NO: 63 - protein, 831 aa, synthetic construct
  source 1..831: note = TALE domain BCL11A #14
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 64 - DNA, 19 bp, synthetic construct
  source 1..19: mol_type = other DNA; note = BCL11A gene binding site
tctcttgtccacagctcgg

SEQ ID NO: 65 - protein, 831 aa, synthetic construct
  source 1..831: note = TALE domain BCL11A #15
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALESIVAQLSRPDPALAALTNDHLVALACLGGRPALDAVKKGLPHAPALIKRTNRRIPERTSHRVAGSGGG

SEQ ID NO: 66 - DNA, 19 bp, synthetic construct
  source 1..19: mol_type = other DNA; note = BCL11A gene binding site
tctcccgctgactgcgcct

SEQ ID NO: 67 - protein, 831 aa, synthetic construct
  source 1..831: note = TALE domain BCL11A #16
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALESIVAQLSRPDPALAALTNDHLVALACLGGRPALDAVKKGLPHAPALIKRTNRRIPERTSHRVAGSGGG

SEQ ID NO: 68 - DNA, 19 bp, synthetic construct
  source 1..19: mol_type = other DNA; note = BCL11A gene binding site
tcccttgctgccaaacttt

SEQ ID NO: 69 - protein, 831 aa, synthetic construct
  source 1..831: note = TALE domain BCL11A #17
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 70 - DNA, 19 bp, synthetic construct
  source 1..19: mol_type = other DNA; note = BCL11A gene binding site
tgggccctcacgcctttct

SEQ ID NO: 71 - RNA, 20 nt, synthetic construct
  source 1..20: mol_type = other RNA; note = gRNA #1 against CpG 105, BCL11A gene
gcctttctgcagacgttccc

SEQ ID NO: 72 - RNA, 20 nt, synthetic construct
  source 1..20: mol_type = other RNA; note = gRNA #2 against CpG 105, BCL11A gene
tgggtgtgcgccttggccgg

SEQ ID NO: 73 - RNA, 20 nt, synthetic construct
  source 1..20: mol_type = other RNA; note = gRNA #3 against CpG 105, BCL11A gene
cggtggtgagatgaccgcct

SEQ ID NO: 74 - RNA, 20 nt, synthetic construct
  source 1..20: mol_type = other RNA; note = gRNA #4 against CpG 105, BCL11A gene
ggaatgtgctcacggcgccg

SEQ ID NO: 75 - RNA, 20 nt, synthetic construct
  source 1..20: mol_type = other RNA; note = gRNA #5 against CpG 105, BCL11A gene
gactgcccgcgctttgtcct

SEQ ID NO: 76 - RNA, 20 nt, synthetic construct
  source 1..20: mol_type = other RNA; note = gRNA #6 against CpG 105, BCL11A gene
ccagagtctggcccccggag

SEQ ID NO: 77 - RNA, 20 nt, synthetic construct
  source 1..20: mol_type = other RNA; note = gRNA #7 against CpG 105, BCL11A gene
tctgcgacccttaggagccg

SEQ ID NO: 78 - RNA, 20 nt, synthetic construct
  source 1..20: mol_type = other RNA; note = gRNA #8 against CpG 105, BCL11A gene
gagcgccccgccaagcgact

SEQ ID NO: 79 - RNA, 20 nt, synthetic construct
  source 1..20: mol_type = other RNA; note = gRNA #9 against CpG 105, BCL11A gene
caagtctccaggagcccgcg

SEQ ID NO: 80 - RNA, 20 nt, synthetic construct
  source 1..20: mol_type = other RNA; note = gRNA #10 against CpG 105, BCL11A gene
cgcggaatccagcctaagtt

SEQ ID NO: 81 - RNA, 20 nt, synthetic construct
  source 1..20: mol_type = other RNA; note = gRNA #11 against CpG 105, BCL11A gene
cccgctgcggagctgtaact

SEQ ID NO: 82 - RNA, 20 nt, synthetic construct
  source 1..20: mol_type = other RNA; note = gRNA #1 against CpG 31, BCL11A gene
cgctcctgagtccgcggagt

SEQ ID NO: 83 - RNA, 20 nt, synthetic construct
  source 1..20: mol_type = other RNA; note = gRNA #2 against CpG 31, BCL11A gene
cacggctctccccgtcgccg

SEQ ID NO: 84 - RNA, 20 nt, synthetic construct
  source 1..20: mol_type = other RNA; note = gRNA #3 against CpG 31, BCL11A gene
ccgccttttgttccggccag

SEQ ID NO: 85 - RNA, 20 nt, synthetic construct
  source 1..20: mol_type = other RNA; note = gRNA #4 against CpG 31, BCL11A gene
gcgcgaggagccggcacaaa

SEQ ID NO: 86 - RNA, 20 nt, synthetic construct
  source 1..20: mol_type = other RNA; note = gRNA #5 against CpG 31, BCL11A gene
gccactttctcactattgtg

SEQ ID NO: 87 - RNA, 20 nt, synthetic construct
  source 1..20: mol_type = other RNA; note = gRNA #6 against CpG 31, BCL11A gene
gctgcctctgaggttcggtc

SEQ ID NO: 88 - RNA, 20 nt, synthetic construct
  source 1..20: mol_type = other RNA; note = gRNA #7 against CpG 31, BCL11A gene
aagggcaggagctagggccg

SEQ ID NO: 89 - RNA, 20 nt, synthetic construct
  source 1..20: mol_type = other RNA; note = gRNA #8 against CpG 31, BCL11A gene
gagcccggactgctgcctcc

SEQ ID NO: 90 - RNA, 20 nt, synthetic construct
  source 1..20: mol_type = other RNA; note = gRNA #1 against CpG 38, BCL11A gene
gtttacaagcaccgcgtgtg

SEQ ID NO: 91 - RNA, 20 nt, synthetic construct
  source 1..20: mol_type = other RNA; note = gRNA #2 against CpG 38, BCL11A gene
aacagacagaggaccgagcg

SEQ ID NO: 92 - RNA, 20 nt, synthetic construct
  source 1..20: mol_type = other RNA; note = gRNA #3 against CpG 38, BCL11A gene
ggcgccgggtgggcgatccg

SEQ ID NO: 93 - RNA, 20 nt, synthetic construct
  source 1..20: mol_type = other RNA; note = gRNA #4 against CpG 38, BCL11A gene
ggtcgggcaaggcccgggcg

SEQ ID NO: 94 - RNA, 20 nt, synthetic construct
  source 1..20: mol_type = other RNA; note = gRNA #5 against CpG 38, BCL11A gene
aagaggtctcggcattgtgc

SEQ ID NO: 95 - RNA, 22 nt, synthetic construct
  source 1..22: mol_type = other RNA; note = gRNA #6 against CpG 38, BCL11A gene
gttccacagcttcgggaccgcg

SEQ ID NO: 96 - RNA, 20 nt, synthetic construct
  source 1..20: mol_type = other RNA; note = gRNA #7 against CpG 38, BCL11A gene
gaaatcggctgggtgaaact

SEQ ID NO: 97 - RNA, 20 nt, synthetic construct
  source 1..20: mol_type = other RNA; note = gRNA #8 against CpG 38, BCL11A gene
gcagtgtctccgcgccagcc

SEQ ID NO: 98 - RNA, 23 nt, synthetic construct
  source 1..23: mol_type = other RNA; note = gRNA #9 against CpG 38, BCL11A gene
cctcccctcccctccgccctggg

SEQ ID NO: 99 - RNA, 23 nt, synthetic construct
  source 1..23: mol_type = other RNA; note = gRNA #1 against CpG 115, BCL11A gene
tcctcctgtcccggggttaaagg

SEQ ID NO: 100 - RNA, 25 nt, synthetic construct
  source 1..25: mol_type = other RNA; note = gRNA #2 against CpG 115, BCL11A gene
catcttttgggacactctaggctgg

SEQ ID NO: 101 - RNA, 23 nt, synthetic construct
  source 1..23: mol_type = other RNA; note = gRNA #3 against CpG 115, BCL11A gene
aagtcaggcccttcttcggaagg

SEQ ID NO: 102 - RNA, 23 nt, synthetic construct
  source 1..23: mol_type = other RNA; note = gRNA #4 against CpG 115, BCL11A gene
gcagcctggactgcgcgccccgg

SEQ ID NO: 103 - RNA, 20 nt, synthetic construct
  source 1..20: mol_type = other RNA; note = gRNA #5 against CpG 115, BCL11A gene
tgcccggcgattctcgtccg

SEQ ID NO: 104 - RNA, 20 nt, synthetic construct
  source 1..20: mol_type = other RNA; note = gRNA #6 against CpG 115, BCL11A gene
tgagccattcggtcgctagg

SEQ ID NO: 105 - RNA, 20 nt, synthetic construct
  source 1..20: mol_type = other RNA; note = gRNA #7 against CpG 115, BCL11A gene
ggtggtactgaggaccggga

SEQ ID NO: 106 - RNA, 20 nt, synthetic construct
  source 1..20: mol_type = other RNA; note = gRNA #1 for IFNAR1 gene
aggaacggcgcgtgcgcgga

SEQ ID NO: 107 - RNA, 20 nt, synthetic construct
  source 1..20: mol_type = other RNA; note = gRNA #8 against CpG 115, BCL11A gene
attttctgggtgctcagagg

SEQ ID NO: 108 - RNA, 20 nt, synthetic construct
  source 1..20: mol_type = other RNA; note = gRNA #9 against CpG 115, BCL11A gene
tggtctcagctcgcgcacgg

SEQ ID NO: 109 - RNA, 20 nt, synthetic construct
  source 1..20: mol_type = other RNA; note = gRNA #10 against CpG 115, BCL11A gene
tggtctcagctcgcgcacgg

SEQ ID NO: 110 - DNA, 23 bp, synthetic construct
  source 1..23: mol_type = other DNA; note = sequence of gRNA targeting exon 3
ggagctctaatccccacgcctgg

SEQ ID NO: 111 - DNA, 23 bp, synthetic construct
  source 1..23: mol_type = other DNA; note = sequence of gRNA for B2M
aggctactagccccatcaagagg

SEQ ID NO: 112 - DNA, 23 bp, synthetic construct
  source 1..23: mol_type = other DNA; note = Primer BIS B2M #1 F
gttgtgttttttggggaagttag

SEQ ID NO: 113 - DNA, 22 bp, synthetic construct
  source 1..22: mol_type = other DNA; note = Primer BIS B2M #1 R
aaaattcctccctatatcctta

SEQ ID NO: 114 - DNA, 27 bp, synthetic construct
  source 1..27: mol_type = other DNA; note = Primer BIS B2M #2 F
aagaatggagaaattttgtagggaatt

SEQ ID NO: 115 - DNA, 25 bp, synthetic construct
  source 1..25: mol_type = other DNA; note = Primer BIS B2M #2 R
accaccaaaaaaaacttaaaaaaaa

SEQ ID NO: 116 - DNA, 25 bp, synthetic construct
  source 1..25: mol_type = other DNA; note = Primer BIS B2M #3 F
tttttttggtttggaggttatttag

SEQ ID NO: 117 - DNA, 25 bp, synthetic construct
  source 1..25: mol_type = other DNA; note = Primer BIS B2M #3 R
caaaacacataaaatccttaacaca

SEQ ID NO: 118 - DNA, 25 bp, synthetic construct
  source 1..25: mol_type = other DNA; note = Primer BIS B2M #4 F
ttttagattggagagttgtggattt

SEQ ID NO: 119 - DNA, 25 bp, synthetic construct
  source 1..25: mol_type = other DNA; note = Primer BIS B2M #4 R
aattttacaactcccctaactaaca

SEQ ID NO: 120 - DNA, 23 bp, synthetic construct
  source 1..23: mol_type = other DNA; note = Primer B2M -241 F
gcaagtcacttagcatctctggg

SEQ ID NO: 121 - DNA, 20 bp, synthetic construct
  source 1..20: mol_type = other DNA; note = Primer B2M -241 R
ttgctgtctgtacatcggcg

SEQ ID NO: 122 - DNA, 20 bp, synthetic construct
  source 1..20: mol_type = other DNA; note = Primer B2M ∧F
tctctcgctccgtgacttcc

SEQ ID NO: 123 - DNA, 21 bp, synthetic construct
  source 1..21: mol_type = other DNA; note = Primer B2M ∧R
cgcttccccgagatccagccc

SEQ ID NO: 124 - DNA, 20 bp, synthetic construct
  source 1..20: mol_type = other DNA; note = Primer B2M +315 F
aggggagacctttggcctac

SEQ ID NO: 125 - DNA, 20 bp, synthetic construct
  source 1..20: mol_type = other DNA; note = Primer B2M +315 R
ctctgacgcttatcgacgcc

SEQ ID NO: 126 - DNA, 23 bp, synthetic construct
  source 1..23: mol_type = other DNA; note = Primer B2M +580 F
agactggagagctgtggacttcg

SEQ ID NO: 127 - DNA, 22 bp, synthetic construct
  source 1..22: mol_type = other DNA; note = Primer B2M +580 R
gccaagcattctacaaacgtcg

SEQ ID NO: 128 - DNA, 22 bp, synthetic construct
  source 1..22: mol_type = other DNA; note = Primer B2M +1529 F
cagtcaggggagctgtaaaacc

SEQ ID NO: 129 - DNA, 22 bp, synthetic construct
  source 1..22: mol_type = other DNA; note = Primer B2M +1529 R
ttgccaggtacttagaaagtgc

SEQ ID NO: 130 - DNA, 21 bp, synthetic construct
  source 1..21: mol_type = other DNA; note = Primer B2M +3307 F
ccttgggttgatccacttagg

SEQ ID NO: 131 - DNA, 23 bp, synthetic construct
  source 1..23: mol_type = other DNA; note = Primer B2M +3307 r
tagtagagtgcctgggacatagc

SEQ ID NO: 132 - DNA, 22 bp, synthetic construct
  source 1..22: mol_type = other DNA; note = Primer B2M +3969 F
gtgtctgggtttcatccatccg

SEQ ID NO: 133 - DNA, 22 bp, synthetic construct
  source 1..22: mol_type = other DNA; note = Primer B2M +3969 R
gctgaaagacaagtctgaatgc

SEQ ID NO: 134 - DNA, 22 bp, synthetic construct
  source 1..22: mol_type = other DNA; note = Primer B2M +5082 F
aggataaaggcaggtggttacc

SEQ ID NO: 135 - DNA, 22 bp, synthetic construct
  source 1..22: mol_type = other DNA; note = Primer B2M +5082 R
agatgtccaatgtggaaatggc

SEQ ID NO: 136 - DNA, 23 bp, synthetic construct
  source 1..23: mol_type = other DNA; note = Primer CCR5 -305 F
agtctgactacagaggccactgg

SEQ ID NO: 137 - DNA, 23 bp, synthetic construct
  source 1..23: mol_type = other DNA; note = Primer CCR5 R -255
aggcaaatgagaccccaaacagc

SEQ ID NO: 138 - DNA, 23 bp, synthetic construct
  source 1..23: mol_type = other DNA; note = Primer PPP1R12C -861 F
taagaaccgaggacaagtagtgc

SEQ ID NO: 139 - DNA, 22 bp, synthetic construct
  source 1..22: mol_type = other DNA; note = Primer PPP1R12C -768R
tgctgggatgacgagcgtaagc

SEQ ID NO: 140 - protein, 149 aa, Homo sapiens
NVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 141 - protein, 250 aa, Homo sapiens
RLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 142 - protein, 136 aa, Homo sapiens
MFPQSRHPTPHQAAGQPFKFTIPESLDRIKEEFQFLQAQYHSLKLECEKLASEKTEMQRHYVMYYEMSYGLNIEMHKQTEIAKRLNTICAQVIPFLSQEHQQQVAQAVERAKQVTMAELNAIIGQQQLQAQHLSHG

SEQ ID NO: 143 - protein, 308 aa, Homo sapiens
LNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 144 - protein, 579 aa, Homo sapiens
LEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 145 - protein, 279 aa, Homo sapiens
MGPDRVTARELCENDDLATSLVLDPYLGFRTHKMNVSPVPPLRRQQHLRSALETFLRQRDLEAAYRALTLGGWTARYFQSRGPRQEAALKTHVYRYLRAFLPESGFTILPCTRYSMETNGAKIVSTRAWKKNEKLELLVGCIAELREADEGLLRAGENDFSIMYSTRKRSAQLWLGPAAFINHDCKPNCKFVPADGNAACVKVLRDIEPGDEVTCFYGEGFFGEKNEHCECHTCERKGEGAFRTRPREPALPPRPLDKYQLRETKRRLQQGLDSGSRQG

SEQ ID NO: 146 - protein, 191 aa, Homo sapiens
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 147 - protein, 831 aa, synthetic construct
  source 1..831: note = TALE domain
MGKPIPNPLLGLDSTGGMAPKKKRKVDGGVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSGGG

SEQ ID NO: 148 - DNA, 19 bp, synthetic construct
  source 1..19: mol_type = other DNA; note = Binding site
tgctcgcgctactctctct

SEQ ID NO: 149 - RNA, 20 nt, synthetic construct
  source 1..20: mol_type = other RNA; note = guide RNA (gRNA) #1 for beta2-microglobulin gene
tataagtggaggcgtcgcgc

SEQ ID NO: 150 - RNA, 20 nt, synthetic construct
  source 1..20: mol_type = other RNA; note = gRNA #2 for beta2-microglobulin gene
gcccgaatgctgtcagcttc

SEQ ID NO: 151 - RNA, 20 nt, synthetic construct
  source 1..20: mol_type = other RNA; note = gRNA #3 for beta2-microglobulin gene
tgcgtcgctggcttggagac

SEQ ID NO: 152 - RNA, 20 nt, synthetic construct
  source 1..20: mol_type = other RNA; note = gRNA #4 for beta2-microglobulin gene
ccaatcaggacaaggcccgc

SEQ ID NO: 153 - RNA, 20 nt, synthetic construct
  source 1..20: mol_type = other RNA; note = gRNA #5 for beta2-microglobulin gene
agggtaggagagactcacgc

SEQ ID NO: 154 - RNA, 20 nt, synthetic construct
  source 1..20: mol_type = other RNA; note = gRNA #6 for beta2-microglobulin gene
gcgggccaccaaggagaact

SEQ ID NO: 155 - RNA, 20 nt, synthetic construct
  source 1..20: mol_type = other RNA; note = gRNA #7 for beta2-microglobulin gene
gctactctctctttctggcc

SEQ ID NO: 156 - RNA, 20 nt, synthetic construct
  source 1..20: mol_type = other RNA; note = gRNA #8 for beta2-microglobulin gene
ctcccgctctgcaccctctg

SEQ ID NO: 157 - RNA, 20 nt, synthetic construct
  source 1..20: mol_type = other RNA; note = gRNA #9 for beta2-microglobulin gene
tttggcctacggcgacggga

SEQ ID NO: 158 - RNA, 20 nt, synthetic construct
  source 1..20: mol_type = other RNA; note = gRNA #10 for beta2-microglobulin gene
ggggcaagtagcgcgcgtcc

SEQ ID NO: 159 - RNA, 20 nt, synthetic construct
  source 1..20: mol_type = other RNA; note = gRNA #11 for beta2-microglobulin gene
tagtccagggctggatctcg

SEQ ID NO: 160 - RNA, 88 nt, synthetic construct
  source 1..88: mol_type = other RNA; note = gRNA scaffold
gtttaagagctatgctggaaacagcatagcaagtttaaataaggctagtccgttattcaacttgaaaaagtggcaccgagtcggtgct

SEQ ID NO: 161 - RNA, 20 nt, synthetic construct
  source 1..20: mol_type = other RNA; note = gRNA #2 for IFNAR1 gene
aagaggcggcgcgtgcgtag

SEQ ID NO: 162 - RNA, 20 nt, synthetic construct
  source 1..20: mol_type = other RNA; note = gRNA #3 for IFNAR1 gene
gggcggtgtgacttaggacg

SEQ ID NO: 163 - RNA, 20 nt, synthetic construct
  source 1..20: mol_type = other RNA; note = gRNA #4 for IFNAR1 gene
ccagatgatggtcgtcctcc

SEQ ID NO: 164 - RNA, 20 nt, synthetic construct
  source 1..20: mol_type = other RNA; note = gRNA #5 for IFNAR1 gene
gaccctagtgctcgtcgccg

SEQ ID NO: 165 - RNA, 20 nt, synthetic construct
  source 1..20: mol_type = other RNA; note = gRNA #6 for IFNAR1 gene
tgggtgttgtccgcagccgc

SEQ ID NO: 166 - RNA, 20 nt, synthetic construct
  source 1..20: mol_type = other RNA; note = gRNA #7 for IFNAR1 gene
acgggggcggcgatgctgtt

SEQ ID NO: 167 - RNA, 20 nt, synthetic construct
  source 1..20: mol_type = other RNA; note = gRNA #8 for IFNAR1 gene
gaccgaaggtttcccagact

SEQ ID NO: 168 - RNA, 20 nt, synthetic construct
  source 1..20: mol_type = other RNA; note = gRNA #9 for IFNAR1 gene
gtcgggtttaatctttggcg

SEQ ID NO: 169 - RNA, 20 nt, synthetic construct
  source 1..20: mol_type = other RNA; note = gRNA #10 for IFNAR1 gene
cgctcccgaggacccgtaca

SEQ ID NO: 170 - RNA, 20 nt, synthetic construct
  source 1..20: mol_type = other RNA; note = gRNA #11 for IFNAR1 gene
cgggtcccacccccgtgaaa

SEQ ID NO: 171 - RNA, 20 nt, synthetic construct
  source 1..20: mol_type = other RNA; note = gRNA #12 for IFNAR1 gene
tcaaactcgacacaaagctc

SEQ ID NO: 172 - RNA, 20 nt, synthetic construct
  source 1..20: mol_type = other RNA; note = gRNA #13 for IFNAR1 gene
gcggagccgcggtactttcc

SEQ ID NO: 173 - RNA, 20 nt, synthetic construct
  source 1..20: mol_type = other RNA; note = gRNA #1 for VEGFA gene
ggcgcgcgcgctaggtggga

SEQ ID NO: 174 - RNA, 20 nt, synthetic construct
  source 1..20: mol_type = other RNA; note = gRNA #2 for VEGFA gene
agagaggctcaccgcccacg

SEQ ID NO: 175 - RNA, 20 nt, synthetic construct
  source 1..20: mol_type = other RNA; note = gRNA #3 for VEGFA gene
gtacgtgcggtgactccggt

(19) United States
(12) Patent Application Publication
(10) Pub. No.: US 2025/0236870 A1
(43) Pub. Date: Jul. 24, 2025

(54) PERMANENT EPIGENETIC GENE SILENCING

(71) Applicants: Ospedale San Raffaele S.R.L., Milan (IT); Fondazione Telethon, Rome (IT)

(72) Inventors: Luigi Naldini, Milan (IT); Angelo Leone Lombardo, Milan (IT); Angelo Amabile, Milan (IT); Alessandro Migliara, Milan (IT)

(73) Assignees: Ospedale San Raffaele S.R.L., Milan (IT); Fondazione Telethon, Rome (IT)

(21) Appl. No.: 18/920,508

(22) Filed: Oct. 18, 2024

Related U.S. Application Data
(63) Continuation of application No. 15/521,294, filed on Apr. 23, 2017, now Pat. No. 12,152,240.

(30) Foreign Application Priority Data
Oct. 24, 2014 (GB) 1418965.8

(51) Int. Cl. C12N 15/113; A61K 35/12; A61K 38/00; A61K 38/17; A61K 38/45; A61K 48/00; C07K 14/47; C12N 9/10; C12N 9/22

(52) U.S. Cl. CPC C12N 15/113; A61K 35/12; A61K 38/1709; A61K 38/45; A61K 48/005; C07K 14/4703; C12N 9/1007; C12N 9/22; C12Y 201/01037; C12Y 201/01043; A61K 38/00; C07K 2319/80; C12N 2310/20; C12N 2740/16043; C12N 2800/80

(57) ABSTRACT

A product comprising two or more artificial transcription repressors (ATRs), or polynucleotides encoding therefor, selected from groups (a), (b), (c) or (d): (a) an ATR comprising a DNA-binding domain operably linked to a KRAB domain or homologue thereof; (b) an ATR comprising a DNA-binding domain operably linked to a DNMT3A, DNMT3B or DNMT1 domain or homologue thereof; (c) an ATR comprising a DNA-binding domain operably linked to a DNMT3L domain or homologue thereof; and (d) an ATR comprising a DNA-binding domain operably linked to a SETDB1 domain or homologue thereof, wherein at least two of the ATRs are selected from different groups (a), (b), (c) or (d).

### RELATED APPLICATIONS

This application is a continuation of U.S. application Ser. No. 15/521,294, filed Apr. 23, 2017, which is a national stage filing under 35 U.S.C. § 371 of international application PCT/IB2015/058202, filed Oct. 23, 2015, the entire contents of each of which is incorporated herein by reference in its entirety.

### REFERENCE TO AN ELECTRONIC SEQUENCE LISTING

The contents of the electronic sequence listing (C169870002US02-SEQ-AXW.xml; Size: 210,863 bytes; and Date of Creation: Oct. 17, 2024) is herein incorporated by reference in its entirety.

### FIELD OF THE INVENTION

The present invention relates to gene silencing and/or epigenetic editing. More specifically, the present invention relates to improved methods for silencing a gene of interest or for editing the epigenetic state of a genetic element of interest, including during gene therapy applications.

### BACKGROUND TO THE INVENTION

Gene therapy involves the incorporation of genetic material into a cell to treat or prevent disease. The genetic material may supplement defective genes with functional copies of those genes, inactivate improperly functioning genes or introduce new therapeutic genes to a cell.

A classic example of gene therapy is gene replacement, where a DNA sequence that encodes a functional, therapeutic gene is used to replace a dysfunctional gene (Naldini, L. (2011) Nat. Rev. Genet. 12: 301-15; Kay, M. A. (2011) Nat. Rev. Genet. 12: 316-28; Biffi, A. et al. (2013) Science 341: 1233158; Aiuti, A. et al. (2013) Science 341: 1233151; Aiuti, A. et al. (2009) N. EngL. J. Med. 360: 447-58). However, there are several inherited diseases where the goal of gene therapy is to silence rather than replace gene function. Paradigmatic examples include Huntington's disease, most types of Spinocerebellar ataxias and some collagenopathies. Furthermore, gene silencing 25 is emerging as a promising strategy to treat certain infectious diseases (Younan, P. et al. (2014) MoL. Ther. 22: 257-64), by inactivating either pathogen-associated gene products or host genes that are necessary for the pathogen life cycle.

For example, silencing of the chemokine (C—C motif) receptor type 5 (CCR5) gene, one of two cellular co-receptors required for HIV entry into T cells, has received significant attention. This is because a natural deletion in CCR5 confers resistance to infection by CCR5-tropic HIV strains without causing overt pathological effects (Liu, R. et al. (1996) Cell 86: 367-77; Hutter, G. et al. (2009) N. Engl. J. Med. 360: 692-8).

In addition, it has recently been proposed that the haemoglobinopathies (Weatherall, D. J. (2013) Annu. Rev. Genomics Hum. Genet. 14: 1-24), the most common inherited recessive disorders of the haematopoietic system and major targets for therapeutic gene replacement, can also be amenable to therapeutic gene silencing. This intriguing concept stems from our increasing understanding of the mechanisms that orchestrate the foetal to adult haemoglobin switch during development (Stamatoyannopoulos, G. (2005) Exp. Hematol. 33: 259-71; Bauer, D. E. et al. (2011) Curr. Opin. Pediatr. 23: 1-8) and by extensive clinical evidence showing that persistent expression of the foetal haemoglobin (HbF) significantly ameliorates morbidity and mortality of Sickle Cell Disease (SCD; Platt, O. S. et al. (1994) N. Engl. J. Med. 330: 1639-44) and β-thalassemia (β-Thal; Andreani, M. et al. (2011) Haematologica 96: 128-33) patients. In particular, genome-wide association studies performed on patients affected by the hereditary persistence of HbF revealed that the transcription factor B-cell lymphoma/leukaemia 11A (BCL11A) is a major regulator of the haemoglobin switch (Sankaran, V. G. et al. (2008) Science 322: 1839-42; Uda, M. et al. (2008) Proc. Natl. Acad. Sci. USA 105: 1620-5; Galarneau, G. et al. (2010) Nat. Genet. 42: 1049-51) and that inactivating mutations in this gene result in increased HbF expression (Wilber, A. et al. (2011) Blood 117: 2817-26; Xu, J. et al. (2011) Science 334: 993-6). Moreover, an erythroid-specific enhancer within the second intron of BCL11A has recently been identified (Bauer, D. E. et al. (2013) Science 342: 253-7). Genetic inactivation of this regulatory element impairs BCL11A expression specifically in erythroid precursors, resulting in HbF reactivation, while it preserves the activity of this protein necessary for proper B-cell ontogeny (Canver, M. C. et al. (2015) Nature September 16 doi: 10.1038/nature15521 [Epub ahead of print]; Vierstra, J. et al. (2015) Nat. Methods 12: 927-30).

To date, two main targeting technologies have been used to silence gene expression: RNA interference (RNAi; Davidson, B. L. et al. (2011) Nat. Rev. Genet. 12: 329-40) with single short hairpin RNA (shRNA); and gene targeting with artificial nucleases (AN; Carroll, D. (2014) Annu. Rev. Biochem. 83: 409-39). RNAi exploits the endogenous microRNA (miRNA) pathway to downregulate expression of the target transcript that is complementary to the shRNA (Davidson, B. L. et al. (2011) Nat. Rev. Genet. 12: 329-40). The AN approach exploits the error-prone nature of the non-homologous end joining DNA repair process to permanently disrupt the coding frame of the AN-target gene (Ciccia, A. et al. (2010) Mol. Cell 40: 179-204).

Although promising pre-clinical and clinical data have been obtained using these technologies (DiGiusto, D. L. et al. (2013) Viruses 5: 2898-919; DiGiusto, D. L. et al. (2010) Sci. Transl. Med. 2: 36ra43; Ramachandran, P. S. et al. (2013) Neurotherapeutics 10: 473-85; McBride, J. L. et al. (2011) Mol. Ther. 19: 2152-62), partial depletion of gene expression with shRNA and the low efficiency by which homozygous disruption occurs in diploid mammalian cells may jeopardise efficacy of these treatments. These disadvantages are particularly relevant in those applications where residual levels of gene activity are sufficient for biological function.

Furthermore, safe exploitation of these technologies requires solving issues with: a) off-target gene silencing; b) altering the transcriptional profile of the cell by interfering with the endogenous miRNA pathway; and c) altering the cell cycle progression or triggering apoptosis by over-activating the DNA damage response (Ciccia, A. et al. (2010) Mol. Cell 40: 179-204). In addition, RNAi and AN are not suitable for inactivation of wide non-transcribed regulatory elements, such as promoters or enhancers.

In addition, epigenetic mechanisms have been exploited to silence gene expression. Epigenetics refers to mechanisms that convey heritable changes in the function of the genome without altering the primary DNA sequence. These changes can mediate short-term instructions that can be quickly reverted in response to exogenous stimuli (e.g. histone post-transcriptional modifications; HPTMs). Alternatively, they can constitute long-term instructions that stably contribute to cellular identity and memory (e.g. DNA methylation; Smith, Z. D. et al. (2013) Nat. Rev. Genet. 14: 204-20). Current studies are unravelling the composition and function of the molecular complexes recruited to chromatin to induce epigenetic repressive states, and the mechanisms by which these states are indefinitely propagated throughout cell division (Cedar, H. et al. (2009) Nat. Rev. Genet. 10: 295-304; Chen, T. et al. (2014) Nat. Rev. Genet. 15: 93-106; Probst, A. V. et al. (2009) Nat. Rev. Mol. Cell Biol. 10: 192-206).

A number of studies have established gene silencing using stably expressed artificial transcription repressors (ATRs) created from DNA-binding domains fused to the effector domains of chromatin remodelling enzymes (de Groote, M. L. et al. (2012) Nucleic Acids Res. 40: 10596-613; Mendenhall, E. M. et al. (2013) Nat. Biotechnol. 31:1133-6; Zhang, F. et al. (2011) Nat. Biotechnol. 29: 149-53; Konermann, S. et al. (2013) Nature 500: 472-6; Sera, T. (2009) Adv. Drug Deliv. Rev. 61: 513-26; Qi, L. S. et al. (2013) Cel/152: 1173-83). However, these studies failed to demonstrate permanent epigenetic silencing in the absence of continuous expression of the ATRs, likely because of the intrinsic inability of the chosen effector domains to recreate self-propagating chromatin repressive states at the ATR-target loci.

In addition, silencing induced by artificial Kruppel-associated box (KRAB)-based repressors has been shown to be erased in somatic cells once the repressor proteins are not expressed or no longer bind to their target locus (Szulc, J. et al. (2006) Nat. Methods 3: 109-16).

Accordingly, there remains a significant need for the development of more powerful and safer gene silencing technologies.

### SUMMARY OF THE INVENTION

We have developed a novel approach for gene silencing that exploits endogenous epigenetic mechanisms. Unexpectedly, our approach conveys robust and heritable states of transcriptional repression of the desired target gene. Importantly, this allows permanent inactivation of genes of therapeutic (e.g. disease-causing) or biotechnological interest.

Because of the previous difficulties with sustaining robust gene silencing, and because long-lasting expression of artificial transcription repressors (ATRs) from integrating vectors may represent a major safety threat to the cells, we selected to use only ATRs that satisfy all of the following criteria:
1. work by combinatorial assembly of two or more different effector modules;
2. establish robust and permanent states of epigenetic repression; and
3. exert this biological function when transiently expressed in the cell.

This approach has allowed us to improve both the efficiency and safety of gene silencing, as activity of each individual ATR at off-target sites will be transient if not absent.

In one aspect, the present invention provides a product comprising two or more artificial transcription repressors (ATRs), or polynucleotides encoding therefor, selected from groups (a), (b), (c) or (d):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof;
(c) an ATR comprising a DNA-binding domain operably linked to a DNMT3L domain or homologue thereof; and
(d) an ATR comprising a DNA-binding domain operably linked to a SETDB1 domain or homologue thereof
   wherein at least two of the ATRs are selected from different groups (a), (b), (c) or (d).

In another aspect, the present invention provides a product comprising two or more artificial transcription repressors (ATRs), or polynucleotides encoding therefor, selected from groups (a), (b) or (c):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof; and
(c) an ATR comprising a DNA-binding domain operably linked to a DNMT3L domain or homologue thereof
   wherein at least two of the ATRs are selected from different groups (a), (b) or (c).

In one embodiment, the product of the invention comprises the ATRs (a) and (b), or polynucleotides encoding therefor. In another embodiment, the product of the invention comprises the ATRs (a) and (c), or polynucleotides encoding therefor. In another embodiment, the product of the invention comprises the ATRs (b) and (c), or polynucleotides encoding therefor. In a preferred embodiment, the product of the invention comprises the ATRs (a), (b) and (c), or polynucleotides encoding therefor. In another embodiment, the product of the invention comprises the ATRs (a), (b) and (d), or polynucleotides encoding therefor. In another embodiment, the product of the invention comprises the ATRs (b) and (d), or polynucleotides encoding therefor. In another embodiment, the product of the invention comprises the ATRs (c) and (d), or polynucleotides encoding therefor. In another preferred embodiment, the product of the invention comprises the ATRs (b), (c) and (d), or polynucleotides encoding therefor.

The KRAB domain or homologue thereof may comprise an amino acid sequence that has at least 50%, 60%, 70%, 80%, 90%, 95%, 99% or 100% identity to SEQ ID NO: 1, 2, 3, 4, 5, 6 or 7 wherein the amino acid sequence substantially retains the natural function of the protein represented by SEQ ID NO: 1, 2, 3, 4, 5, 6, or 7.

The DNMT3A domain or homologue thereof may comprise an amino acid sequence that has at least 50%, 60%, 70%, 80%, 90%, 95%, 99% or 100% identity to SEQ ID NO: 8 wherein the amino acid sequence substantially retains the natural function of the protein represented by SEQ ID NO: 8.

The DNMT3B domain or homologue thereof may comprise an amino acid sequence that has at least 50%, 60%, 70%, 80%, 90%, 95%, 99% or 100% identity to SEQ ID NO: 9 or 36 wherein the amino acid sequence substantially retains the natural function of the protein represented by SEQ ID NO: 9 or 36.

The DNMT1 domain or homologue thereof may comprise an amino acid sequence that has at least 50%, 60%, 70%, 80%, 90%, 95%, 99% or 100% identity to SEQ ID NO: 10 wherein the amino acid sequence substantially retains the natural function of the protein represented by SEQ ID NO: 10.

The DNMT3L domain or homologue thereof may comprise an amino acid sequence that has at least 50%, 60%, 70%, 80%, 90%, 95%, 99% or 100% identity to SEQ ID NO: 11 wherein the amino acid sequence substantially retains the natural function of the protein represented by SEQ ID NO: 11.

The SETDB1 domain or homologue thereof may comprise an amino acid sequence that has at least 50%, 60%, 70%, 80%, 90%, 95%, 99% or 100% identity to SEQ ID NO: 12 or 13 wherein the amino acid sequence substantially retains the natural function of the protein represented by SEQ ID NO: 12 or 13.

In one embodiment, the DNA-binding domain of (a), (b), (c) or (d) comprises a domain independently selected from a TALE DNA-binding domain, a zinc finger domain, a tetR DNA-binding domain, a meganuclease or a CRISPR/Cas system. In a preferred embodiment, the DNA-binding domain of (a), (b), (c) or (d) comprises a TALE DNA-binding domain or a CRISPR/Cas system.

The DNA-binding domains, for example the TALE DNA-binding domains or the CRISPR/Cas system, of (a), (b), (c) or (d) may be selected or engineered to bind to different binding sites.

The DNA-binding domains may bind to binding sites within a target gene or within regulatory sequences for the target gene, for example promoter or enhancer sequences.

The DNA-binding domains may bind to binding sites within splicing sites. Splicing variants of a given gene may be regulated by DNA methylation/demethylation at splicing sites. In turn, these modifications may cause exon exclusion/inclusion in the mature transcript. This exclusion/inclusion may have therapeutic relevance, such as in the case of Duchenne Muscular Dystrophy, in which exclusion (by genetic ablation or exon skipping) from the mature mRNA of an exon bearing the most frequent disease-causing mutation has been proposed for therapy (Ousterout, D. G. et al. (2015) Mol. Ther. 23: 523-32; Ousterout, D. G. et al. (2015) Nat. Commun. 6: 6244; Kole, R. et al. (2015) Adv. Drug Deliv. Rev. 87: 104-7; Touznik, A. et al. (2014) Expert Opin. Biol. Ther. 14: 809-19).

The ATRs of the present invention may also target genetic elements which may be actively transcribed or not (e.g. sequences that control the topological arrangement, stability and replication of the genome, such as insulators, laminin-associated domains, telomeric and centromeric regions), repetitive or mobile elements. Accordingly, the present invention may relate to epigenetic editing, such as silencing/editing of a genetic element. The invention may therefore encompass the use of the products and ATRs of the invention for epigenetic editing of regulatory DNA elements, such as those described herein. Epigenetic editing of a target gene or of a genetic element may also be associated with its transcription activation or activity, respectively. The invention may also encompass the use of the products and ATRs of the invention for simultaneous epigenetic silencing of multiple target genes or regulatory DNA elements, such as those described herein.

In one embodiment, the polynucleotides encoding the two or more ATRs are in the form of a single vector or are comprised within separate vectors.

In one embodiment where two ATRs are used, polynucleotides encoding (a) and (b) may be comprised within a single vector; polynucleotides encoding (a) and (c) may be comprised within a single vector; or polynucleotides encoding (b) and (c) may be comprised within a single vector.

In another embodiment where two ATRs are used, polynucleotides encoding (a) and (d) may be comprised within a single vector; polynucleotides encoding (b) and (d) may be comprised within a single vector; or polynucleotides encoding (c) and (d) may be comprised within a single vector.

In another embodiment where two ATRs are used, polynucleotides encoding (a) and (b) may be comprised within separate vectors; polynucleotides encoding (a) and (c) may be comprised within separate vectors; or polynucleotides encoding (b) and (c) may be comprised within separate vectors.

In another embodiment where two ATRs are used, polynucleotides encoding (a) and (d) may be comprised within separate vectors; polynucleotides encoding (b) and (d) may be comprised within separate vectors; or polynucleotides encoding (c) and (d) may be comprised within separate vectors.

In one embodiment where three ATRs are used, polynucleotides encoding (a), (b) and (c) may be comprised within a single vector; polynucleotides encoding (a), (b) and (c) may be comprised within separate vectors; polynucleotides encoding (a) and (b) may be comprised within a single vector and the polynucleotide encoding (c) may be comprised within a separate vector; polynucleotides encoding (a) and (c) may be comprised within a single vector and the polynucleotide encoding (b) may be comprised within a separate vector; or polynucleotides encoding (b) and (c) may be comprised within a single vector and the polynucleotide encoding (a) may be comprised within a separate vector.

In another embodiment where three ATRs are used, polynucleotides encoding (a), (b) and (d) may be comprised within a single vector; polynucleotides encoding (a), (b) and (d) may be comprised within separate vectors; polynucleotides encoding (a) and (b) may be comprised within a single vector and the polynucleotide encoding (d) may be comprised within a separate vector; polynucleotides encoding (a) and (d) may be comprised within a single vector and the polynucleotide encoding (b) may be comprised within a separate vector; or polynucleotides encoding (b) and (d) may be comprised within a single vector and the polynucleotide encoding (a) may be comprised within a separate vector.

In another embodiment where three ATRs are used, polynucleotides encoding (b), (c) and (d) may be comprised within a single vector; polynucleotides encoding (b), (c) and (d) may be comprised within separate vectors; polynucleotides encoding (b) and (c) may be comprised within a single vector and the polynucleotide encoding (d) may be comprised within a separate vector; polynucleotides encoding (b) and (d) may be comprised within a single vector and the polynucleotide encoding (c) may be comprised within a separate vector; or polynucleotides encoding (c) and (d) may be comprised within a single vector and the polynucleotide encoding (b) may be comprised within a separate vector.

The vectors may, for example, be plasmid vectors, mRNA vectors (e.g. in vitro transcribed mRNA vectors) or viral vectors. Preferably the vectors enable transient expression of the ATRs within a cell.

As an alternative to the delivery of polynucleotides encoding ATRs to cells, the ATRs of the present invention may be delivered to cells by protein transduction. The protein transduction may, for example, be via vector delivery or by direct protein delivery.

In one embodiment, the product of the invention is in the form of a pharmaceutical composition further comprising a pharmaceutically acceptable carrier, diluent or excipient.

In one embodiment, the product of the invention further comprises a KRAB domain or homologue thereof, or polynucleotide encoding therefor, wherein the KRAB domain or homologue thereof is not operably linked to a DNA-binding domain.

In one embodiment, the product of the invention further comprises a DNMT3A, DNMT3B or DNMT1 domain or homologue thereof, or polynucleotide encoding therefor, wherein the DNMT3A, DNMT3B or DNMT1 domain or homologue thereof is not operably linked to a DNA-binding domain.

In one embodiment, the product of the invention further comprises a DNMT3L domain or homologue thereof, or polynucleotide encoding therefor, wherein the DNMT3L domain or homologue thereof is not operably linked to a DNA-binding domain.

In one embodiment, the product of the invention further comprises a SETDB1 domain or homologue thereof, or polynucleotide encoding therefor, wherein the SETDB1 domain or homologue thereof is not operably linked to a DNA-binding domain.

In another aspect, the present invention provides the product of the invention for use in therapy.

In another aspect, the present invention provides the product of the invention for use in therapy, wherein the two or more artificial transcription repressors (ATRs), or polynucleotides encoding therefor, are a combined preparation for administration to a subject simultaneously, sequentially or separately.

Herein, administration to a subject may include administration to a cell, for example during ex vivo therapy.

In another aspect, the present invention provides the use of the product of the invention for silencing a target gene. The use may, for example, be in vitro or ex vivo use. For example, a target gene may be silenced in a population of cells (e.g. a cell line or primary cells) to enhance the production of an agent (e.g. a biotherapeutic agent) by the cells, or to impart a growth advantage to the cells. Alternatively, for example, a target gene may be silenced to generate a knockout animal model for the target gene. The epigenetic approach of the present invention provides an alternative to existing methods of knocking out a gene, such as those utilising homologous recombination. Alternatively, for example, a target gene may be silenced in a plant cell.

According to the above uses, including the uses in therapy, the delivery of the two or more ATRs of the invention to a cell may silence a target gene. The delivery may be transient delivery. The delivery may be via expression of the two or more ATRs in a cell, for example expression from polynucleotides encoding the ATRs. The delivery of the two or more ATRs of the invention to a cell may also cause exon exclusion/inclusion in a mature transcript, for example through an effect on a splicing site. The delivery of the two or more ATRs of the invention to a cell may also enable silencing and/or editing of a genetic element as described herein.

In one embodiment, expression of the two or more ATRs of the invention in a cell silences a target gene. The expression may be transient expression.

In one embodiment, delivery of the two or more ATRs of the invention to a cell (e.g. by expression in the cell) permanently silences a target gene. In another embodiment, delivery of the two or more ATRs of the invention to a cell (e.g. by expression in the cell) permanently silences a target gene in the cell's progeny. For example, the cell may be a stem cell and the target gene may be silenced in the stem cell's progeny (e.g. the target gene may be silenced in cells resulting from differentiation of the stem cells).

By way of example, the cells may be derived from animals (such as mammals, e.g. humans), fungi (such as yeast) or plants. For example, the cells may be haematopoietic stem and progenitor cells, T lymphocytes, mesenchymal stem cells, fibroblasts, monocytes, epidermal or neural stem cells.

The separation of the binding sites to which the DNA-binding domains of the different ATRs are selected to bind is not particularly limited in size. For example, the DNA-binding domains of the different ATRs may be selected to bind to binding sites that are separated by about 1-100 bp, 1-50 bp, 1-30 bp, 5-30 bp, 10-30 bp or 15-30 bp. In one embodiment, the DNA-binding domains of the different ATRs are selected to bind to binding sites that are separated by 1-30 bp. Preferably, the DNA-binding domains of the different ATRs are selected to bind to binding sites that are separated by about 15-25 bp. For example, the DNA-binding domains of the different ATRs may be selected to bind to binding sites that are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 bp.

The DNA-binding domains of the different ATRs may also be selected to bind to the same binding site, for example the DNA-binding domains of the different ATRs may be selected to bind to binding sites that are separated by 0 bp. Thus, for example, the DNA-binding domains of the different ATRs may be selected to bind to binding sites that are separated by about 0-100 bp, 0-50 bp, 0-30 bp, 5-30 bp, 10-30 bp or 15-30 bp. The DNA-binding domains of the different ATRs may be selected to bind to binding sites that are separated by about 0-15 or 15-25 bp.

The directional order in which the different ATRs bind relative to the target gene is not particularly important. In one embodiment, the two or more ATRs comprise an ATR comprising a KRAB domain or homologue thereof and an ATR comprising a DNMT3A, DNMT3B or DNMT1 domain or homologue thereof, and the DNA-binding domains (e.g. TALE DNA-binding domains) of each ATR are selected such that the ATR comprising a DNMT3A, DNMT3B or DNMT1 domain or homologue thereof binds to DNA upstream of the ATR comprising a KRAB domain or homologue thereof.

In one embodiment, the DNA-binding domains are TALE DNA-binding domains or CRISPR/Cas systems.

The selection of the DNA-binding domains may comprise engineering DNA-binding domains to bind to specific, desired DNA sequences.

In another aspect, the present invention provides an artificial transcription repressor (ATR) comprising a DNA-binding domain operably linked to a KRAB domain or homologue thereof, or polynucleotide encoding therefor, for use in therapy wherein the ATR is administered to a subject simultaneously, sequentially or separately in combination with a second ATR comprising a DNA-binding domain operably linked to a DNMT3A, DNMT3B or DNMT1 domain or homologue thereof and/or a third ATR comprising a DNA-binding domain operably linked to a DNMT3L domain or homologue thereof, or polynucleotides encoding therefor.

In another aspect, the present invention provides an artificial transcription repressor (ATR) comprising a DNA-binding domain operably linked to a DNMT3A, DNMT3B or DNMT1 domain or homologue thereof, or polynucleotide encoding therefor, for use in therapy wherein the ATR is administered to a subject simultaneously, sequentially or separately in combination with a second ATR comprising a DNA-binding domain operably linked to a KRAB domain or homologue thereof and/or a third ATR comprising a DNA-binding domain operably linked to a DNMT3L domain or homologue thereof, or polynucleotides encoding therefor.

In another aspect, the present invention provides an artificial transcription repressor (ATR) comprising a DNA-binding domain operably linked to a DNMT3L domain or homologue thereof, or polynucleotide encoding therefor, for use in therapy wherein the ATR is administered to a subject simultaneously, sequentially or separately in combination with a second ATR comprising a DNA-binding domain operably linked to a KRAB domain or homologue thereof and/or a third ATR comprising a DNA-binding domain operably linked to a DNMT3A, DNMT3B or DNMT1 domain or homologue thereof, or polynucleotides encoding therefor.

In another aspect, the present invention provides an artificial transcription repressor (ATR) comprising a DNA-binding domain operably linked to a KRAB domain or homologue thereof, or polynucleotide encoding therefor, for use in therapy wherein the ATR is administered to a subject simultaneously, sequentially or separately in combination with a second ATR comprising a DNA-binding domain operably linked to a DNMT3A, DNMT3B or DNMT1 domain or homologue thereof, and/or a third ATR comprising a DNA-binding domain operably linked to a DNMT3L domain or homologue thereof, and/or a fourth ATR comprising a DNA-binding domain operably linked to a SETDB1 domain or homologue thereof, or polynucleotides encoding therefor.

In another aspect, the present invention provides an artificial transcription repressor (ATR) comprising a DNA-binding domain operably linked to a DNMT3A, DNMT3B or DNMT1 domain or homologue thereof, or polynucleotide encoding therefor, for use in therapy wherein the ATR is administered to a subject simultaneously, sequentially or separately in combination with a second ATR comprising a DNA-binding domain operably linked to a KRAB domain or homologue thereof, and/or a third ATR comprising a DNA-binding domain operably linked to a DNMT3L domain or homologue thereof, and/or a fourth ATR comprising a DNA-binding domain operably linked to a SETDB1 domain or homologue thereof, or polynucleotides encoding therefor.

In another aspect, the present invention provides an artificial transcription repressor (ATR) comprising a DNA-binding domain operably linked to a DNMT3L domain or homologue thereof, or polynucleotide encoding therefor, for use in therapy wherein the ATR is administered to a subject simultaneously, sequentially or separately in combination with a second ATR comprising a DNA-binding domain operably linked to a KRAB domain or homologue thereof, and/or a third ATR comprising a DNA-binding domain operably linked to a DNMT3A, DNMT3B or DNMT1 domain or homologue thereof, and/or a fourth ATR comprising a DNA-binding domain operably linked to a SETDB1 domain or homologue thereof, or polynucleotides encoding therefor.

In another aspect, the present invention provides an artificial transcription repressor (ATR) comprising a DNA-binding domain operably linked to a SETDB1 domain or homologue thereof, or polynucleotide encoding therefor, for use in therapy wherein the ATR is administered to a subject simultaneously, sequentially or separately in combination with a second ATR comprising a DNA-binding domain operably linked to a DNMT3A, DNMT3B or DNMT1 domain or homologue thereof, and/or a third ATR comprising a DNA-binding domain operably linked to a DNMT3L domain or homologue thereof, and/or a fourth ATR comprising a DNA-binding domain operably linked to a KRAB domain or homologue thereof, or polynucleotides encoding therefor.

In another aspect, the present invention provides a cell comprising the two or more artificial transcription repressors (ATRs) of the invention. The cell may be transfected by the polynucleotides encoding the two or more ATRs of the invention. The polynucleotides may be in the form of a single vector or may be comprised within separate vectors.

In another aspect, the present invention provides a cell wherein said cell is a descendant of a cell comprising the two or more artificial transcription repressors (ATRs) of the invention. In one embodiment, the descendant cell no longer comprises the two or more ATRs of the invention. In another aspect, the present invention provides the cell of the invention for use in therapy.

In another aspect, the present invention provides a method of gene therapy comprising transfecting a cell with the polynucleotides encoding the two or more artificial transcription repressors (ATRs) of the invention, wherein the polynucleotides are in the form of a single vector or are comprised within separate vectors.

In one embodiment, the transfection is carried out ex vivo.

In another aspect, the present invention provides a method of gene therapy comprising administering two or more artificial transcription repressors (ATRs), or polynucleotides encoding therefor, selected from groups (a), (b) or (c):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof; and
(c) an ATR comprising a DNA-binding domain operably linked to a DNMT3L domain or homologue thereof
   to a subject simultaneously, sequentially or separately wherein at least two of the ATRs are selected from different groups (a), (b) or (c). The present invention also provides a method of gene therapy comprising administering two or more artificial transcription repressors (ATRs), or polynucleotides encoding therefor, selected from groups (a), (b), (c) or (d):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof;
(c) an ATR comprising a DNA-binding domain operably linked to a DNMT3L domain or homologue thereof; and
(d) an ATR comprising a DNA-binding domain operably linked to a SETDB1 domain or homologue thereof
   to a subject simultaneously, sequentially or separately, wherein at least two of the ATRs are selected from different groups (a), (b), (c) or (d).

In another aspect, the present invention provides a kit comprising two or more artificial transcription repressors (ATRs), or polynucleotides encoding therefor, selected from groups (a), (b) or (c):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof; and
(c) an ATR comprising a DNA-binding domain operably linked to a DNMT3L domain or homologue thereof
   wherein at least two of the ATRs are selected from different groups (a), (b) or (c). The present invention also provides a kit comprising two or more artificial transcription repressors (ATRs), or polynucleotides encoding therefor, selected from groups (a), (b), (c) or (d):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof;
(c) an ATR comprising a DNA-binding domain operably linked to a DNMT3L domain or homologue thereof; and
(d) an ATR comprising a DNA-binding domain operably linked to a SETDB1 domain or homologue thereof
   wherein at least two of the ATRs are selected from different groups (a), (b), (c) or (d).

In another aspect, the present invention provides a method of silencing a target gene comprising the step of administering the two or more ATRs, or polynucleotides encoding therefor, of the invention to a cell. The method may be an in vitro method.

In another aspect, the present invention provides a product comprising an artificial transcription repressor (ATR) comprising a DNA-binding domain operably linked to a DNMT3A, DNMT3B or DNMT1 domain or homologue thereof, preferably a DNMT3A domain or homologue thereof, and an ATR comprising a DNA-binding domain operably linked to a SETDB1 domain or homologue thereof, or polynucleotides encoding therefor. The present invention also provides uses of this product, uses of this product in therapy, cells comprising this product and their descendants, methods employing this product and kits comprising this product, as described herein. This product may also further comprise an ATR comprising a DNA-binding domain operably linked to a DNMT3L or KRAB domain or homologue thereof, or polynucleotide encoding therefor.

In one embodiment, the product comprises an artificial transcription repressor (ATR) comprising a DNA-binding domain operably linked to a DNMT3A, DNMT3B or DNMT1 domain or homologue thereof, preferably a DNMT3A domain or homologue thereof, an ATR comprising a DNA-binding domain operably linked to a SETDB1 domain or homologue thereof, and an ATR comprising a DNA-binding domain operably linked to a DNMT3L domain or homologue thereof, or polynucleotides encoding therefor.

In one embodiment, the product comprises an artificial transcription repressor (ATR) comprising a DNA-binding domain operably linked to a DNMT3A, DNMT3B or DNMT1 domain or homologue thereof, preferably a DNMT3A domain or homologue thereof, an ATR comprising a DNA-binding domain operably linked to a SETDB1 domain or homologue thereof, and an ATR comprising a DNA-binding domain operably linked to a KRAB domain or homologue thereof, or polynucleotides encoding therefor.

The SETDB1 domain or homologue thereof may comprise an amino acid sequence that has at least 50%, 60%, 70%, 80%, 90%, 95%, 99% or 100% identity to SEQ ID NO: 12 or 13 wherein the amino acid sequence substantially retains the natural function of the protein represented by SEQ ID NO: 12 or 13.

In another aspect, the present invention provides an artificial transcription repressor (ATR), or a polynucleotide encoding therefor, wherein the ATR comprises a DNA-binding domain operably linked to two or more domains selected from groups (a), (b) or (c):
(a) a KRAB domain or homologue thereof;
(b) a DNMT3A, DNMT3B or DNMT1 domain or homologue thereof; and
(c) a DNMT3L domain or homologue thereof
   wherein at least two of the domains operably linked to the DNA-binding domain are selected from different groups (a), (b) or (c). The ATR may, for example, comprise a DNA-binding domain operably linked to a domain of group (a), a domain of group (b) and a domain of group (c). The present invention also provides an artificial transcription repressor (ATR) comprising a DNA-binding domain operably linked to two or more domains selected from groups (a), (b), (c) or (d):
(a) a KRAB domain or homologue thereof;
(b) a DNMT3A, DNMT3B or DNMT1 domain or homologue thereof;
(c) a DNMT3L domain or homologue thereof; and
(d) a SETDB1 domain or homologue thereof
   wherein at least two of the domains operably linked to the DNA-binding domain are selected from different groups (a), (b), (c) or (d).

In one embodiment, the DNA-binding domain comprises a TALE DNA-binding domain, a zinc finger domain, a tetR DNA-binding domain, a meganuclease or a CRISPR/Cas system.

The present invention also provides uses of this ATR, uses of this ATR in therapy, cells comprising this ATR and their descendants, methods employing this ATR and kits comprising this ATR, as described herein.

In another aspect, the present invention provides a product comprising two or more different artificial transcription repressors (ATRs), or polynucleotides encoding therefor, wherein the two or more different ATRs individually comprise a DNA-binding domain operably linked to two or more domains selected from groups (a), (b) or (c):
(a) a KRAB domain or homologue thereof;
(b) a DNMT3A, DNMT3B or DNMT1 domain or homologue thereof; and
(c) a DNMT3L domain or homologue thereof
   wherein at least two of the domains operably linked to each individual DNA-binding domain are selected from different groups (a), (b) or (c). Each ATR may, for example, comprise a DNA-binding domain operably linked to a domain of group (a), a domain of group (b) and a domain of group (c). The present invention also provides a product comprising two or more different artificial transcription repressors (ATRs), or polynucleotides encoding therefor, wherein the two or more different ATRs individually comprise a DNA-binding domain operably linked to two or more domains selected from groups (a), (b), (c) or (d):
(a) a KRAB domain or homologue thereof;
(b) a DNMT3A, DNMT3B or DNMT1 domain or homologue thereof;
(c) a DNMT3L domain or homologue thereof; and
(d) a SETDB1 domain or homologue thereof
   wherein at least two of the domains operably linked to each individual DNA-binding domain are selected from different groups (a), (b), (c) or (d).

In one embodiment, the DNA-binding domains of the two or more different ATRs are individually selected from the group consisting of a TALE DNA-binding domain, a zinc finger domain, a tetR DNA-binding domain, a meganuclease or a CRISPR/Cas system.

The DNA-binding domains, for example the TALE DNA-binding domains or the CRISPR/Cas system, of the two or more different ATRs may be selected or engineered to bind to different binding sites.

The DNA-binding domains may bind to binding sites within a target gene or within regulatory sequences for the target gene, for example promoter or enhancer sequences. The DNA-binding domains may bind to binding sites within splicing sites.

The present invention also provides uses of this product, uses of this product in therapy, cells comprising this product and their descendants, methods employing this product and kits comprising this product, as described herein.

In another aspect, the present invention provides a product comprising only one ATR and a separate effector protein that is not operably linked to a DNA-binding domain, or polynucleotides encoding therefor. The ATR may comprise a DNA-binding domain operably linked to an effector domain selected from: (a) a KRAB domain or homologue thereof; (b) a DNMT3A, DNMT3B or DNMT1 domain or homologue thereof; or (c) a DNMT3L domain or homologue thereof (i.e. the ATR may be as described herein). The separate effector protein that is not operably linked to a DNA-binding domain may comprise a KRAB, DNMT3A, DNMT3B, DNMT1 or DNMT3L domain or homologue thereof. The separate effector protein may be an effector domain/protein as described herein. The separate effector protein may be a full-length protein or functional fragment thereof. Preferably the separate effector protein is different to the effector domain of the ATR. Preferably the separate effector protein is of a different class to the effector domain of the ATR. Preferably the separate effector protein is selected such that it does not comprise a domain belonging to the same group (a), (b) or (c) as the effector domain that constitutes the ATR.

The separate effector protein that is not operably linked to a DNA-binding domain may also comprise a SETDB1 domain or homologue thereof.

In another aspect, the present invention provides a product comprising only one ATR and a separate effector protein that is not operably linked to a DNA-binding domain, or polynucleotides encoding therefor. The ATR may comprise a DNA-binding domain operably linked to a SETDB1 effector domain or homologue thereof (i.e. the ATR may be as described herein). The separate effector protein that is not operably linked to a DNA-binding domain may comprise a KRAB, DNMT3A, DNMT3B, DNMT1 or DNMT3L domain or homologue thereof. The separate effector protein may be an effector domain/protein as described herein. The separate effector protein may be a full-length protein or functional fragment thereof.

In one embodiment, the DNA-binding domain of the ATR is selected from the group consisting of a TALE DNA-binding domain, a zinc finger domain, a tetR DNA-binding domain, a meganuclease or a CRISPR/Cas system.

The present invention also provides uses of this product, uses of this product in therapy, cells comprising this product and their descendants, methods employing this product and kits comprising this product, as described herein.

When the product of the invention comprises only one ATR and a separate effector protein that is not operably linked to a DNA-binding domain, the polynucleotides encoding the ATR and separate effector protein may be in the form of a single vector or comprised within separate vectors.

The vectors may, for example, be plasmid vectors, mRNA vectors (e.g. in vitro transcribed mRNA vectors) or viral vectors. Preferably the vectors enable transient expression of the ATR and/or separate effector protein within a cell.

The ATR and/or separate effector protein of the present invention may also be delivered to cells by protein transduction, as described herein.

The ATRs and/or separate effector proteins of the invention, or polynucleotides encoding therefor, may be in the form of a pharmaceutical composition further comprising a pharmaceutically acceptable carrier, diluent or excipient.

In another aspect, the present invention provides the ATR of the invention, or the ATR and separate effector protein of the invention, or polynucleotides encoding therefor, for use in therapy.

In another aspect, the present invention provides the ATR and separate effector protein of the invention, or polynucleotides encoding therefor, for use in therapy, wherein the ATR and separate effector protein, or polynucleotides encoding therefor, are a combined preparation for administration to a subject simultaneously, sequentially or separately.

In another aspect, the present invention provides the ATR of the invention, or the ATR and separate effector protein of the invention, or polynucleotides encoding therefor, for silencing a target gene. The use may, for example, be in vitro or ex vivo use.

According to the above uses, including the uses in therapy, the delivery of the ATR of the invention, or the ATR and separate effector protein of the invention to a cell may silence a target gene. The delivery may be transient delivery. The delivery may be via expression of the ATR of the invention, or the ATR and separate effector protein of the invention in a cell, for example expression from polynucleotides encoding the ATR of the invention, or the ATR and separate effector protein of the invention.

In one embodiment, expression of the ATR of the invention, or the ATR and separate effector protein of the invention in a cell silences a target gene. The expression may be transient expression.

In one embodiment, delivery of the ATR of the invention, or the ATR and separate effector protein of the invention to a cell (e.g. by expression in the cell) permanently silences a target gene. In another embodiment, delivery of the ATR of the invention, or the ATR and separate effector protein of the invention to a cell (e.g. by expression in the cell) permanently silences a target gene in the cell's progeny. For example, the cell may be a stem cell and the target gene may be silenced in the stem cell's progeny (e.g. the target gene may be silenced in cells resulting from differentiation of the stem cells).

By way of example, the cells may be derived from animals (such as mammals, e.g. humans), fungi (such as yeast) or plants. For example, the cells may be haematopoietic stem and progenitor cells, T lymphocytes, mesenchymal stem cells, fibroblasts, monocytes, epidermal or neural stem cells.

In another aspect, the present invention provides an artificial transcription repressor (ATR) comprising a DNA-binding domain operably linked to a KRAB domain or homologue thereof, or polynucleotide encoding therefor, for use in therapy wherein the ATR is administered to a subject simultaneously, sequentially or separately in combination with a first separate effector protein that is not operably linked to a DNA-binding domain comprising a DNMT3A, DNMT3B or DNMT1 domain or homologue thereof and/or a second separate effector protein that is not operably linked to a DNA-binding domain comprising a DNMT3L domain or homologue thereof, or polynucleotides encoding therefor.

In another aspect, the present invention provides an artificial transcription repressor (ATR) comprising a DNA-binding domain operably linked to a DNMT3A, DNMT3B or DNMT1 domain or homologue thereof, or polynucleotide encoding therefor, for use in therapy wherein the ATR is administered to a subject simultaneously, sequentially or separately in combination with a first separate effector protein that is not operably linked to a DNA-binding domain comprising a KRAB domain or homologue thereof and/or a second separate effector protein that is not operably linked to a DNA-binding domain comprising a DNMT3L domain or homologue thereof, or polynucleotides encoding therefor.

In another aspect, the present invention provides an artificial transcription repressor (ATR) comprising a DNA-binding domain operably linked to a DNMT3L domain or homologue thereof, or polynucleotide encoding therefor, for use in therapy wherein the ATR is administered to a subject simultaneously, sequentially or separately in combination with a first separate effector protein that is not operably linked to a DNA-binding domain comprising a KRAB domain or homologue thereof and/or a second separate effector protein that is not operably linked to a DNA-binding domain comprising a DNMT3A, DNMT3B or DNMT1 domain or homologue thereof, or polynucleotides encoding therefor.

A third separate effector protein that is not operably linked to a DNA-binding domain comprising a SETDB1 domain or homologue thereof may also be used in these combinations.

In another aspect, the present invention provides an artificial transcription repressor (ATR) comprising a DNA-binding domain operably linked to a SETDB1 domain or homologue thereof, or polynucleotide encoding therefor, for use in therapy wherein the ATR is administered to a subject simultaneously, sequentially or separately in combination with a first separate effector protein that is not operably linked to a DNA-binding domain comprising a KRAB domain or homologue thereof and/or a second separate effector protein that is not operably linked to a DNA-binding domain comprising a DNMT3A, DNMT3B or DNMT1 domain or homologue thereof and/or a third separate effector protein that is not operably linked to a DNA-binding domain comprising a DNMT3L domain or homologue thereof, or polynucleotides encoding therefor.

In another aspect, the present invention provides a cell comprising the ATR of the invention, or the ATR and separate effector protein of the invention. The cell may be transfected by the polynucleotides encoding the ATR of the invention, or the ATR and separate effector protein of the invention. The polynucleotides encoding the ATR and separate effector protein of the invention may be in the form of a single vector or may be comprised within separate vectors.

In another aspect, the present invention provides a cell wherein said cell is a descendant of a cell comprising the ATR of the invention, or the ATR and separate effector protein of the invention. In one embodiment, the descendant cell no longer comprises the ATR and/or separate effector protein of the invention. In another aspect, the present invention provides the cell of the invention for use in therapy.

In another aspect, the present invention provides a method of gene therapy comprising transfecting a cell with the polynucleotides encoding the ATR of the invention, or the ATR and separate effector protein of the invention. The polynucleotides encoding the ATR and separate effector protein of the invention may be in the form of a single vector or comprised within separate vectors.

In one embodiment, the transfection is carried out ex vivo.

In another aspect, the present invention provides a method of gene therapy comprising administering only one ATR and a separate effector protein that is not operably linked to a DNA-binding domain, or polynucleotides encoding therefor, to a subject simultaneously, sequentially or separately. The ATR may comprise a DNA-binding domain operably linked to an effector domain selected from: (a) a KRAB domain or homologue thereof; (b) a DNMT3A, DNMT3B or DNMT1 domain or homologue thereof; or (c) a DNMT3L domain or homologue thereof (i.e. the ATR may be as described herein). The separate effector protein that is not operably linked to a DNA-binding domain may comprise a KRAB, DNMT3A, DNMT3B, DNMT1 or DNMT3L domain or homologue thereof. The separate effector protein may be an effector domain/protein as described herein. The separate effector protein may be a full-length protein or functional fragment thereof. Preferably the separate effector protein is different to the effector domain of the ATR. Preferably the separate effector protein is of a different class to the effector domain of the ATR. Preferably the separate effector protein is selected such that it does not comprise a domain belonging to the same group (a), (b) or (c) as the effector domain that constitutes the ATR.

The ATR may also comprise a DNA-binding domain operably linked to a SETDB1 effector domain or homologue thereof. The separate effector protein that is not operably linked to a DNA-binding domain may also comprise a SETDB1 domain or homologue thereof.

In another aspect, the present invention provides a kit comprising only one ATR and a separate effector protein that is not operably linked to a DNA-binding domain, or polynucleotides encoding therefor. The ATR may comprise a DNA-binding domain operably linked to an effector domain selected from: (a) a KRAB domain or homologue thereof; (b) a DNMT3A, DNMT3B or DNMT1 domain or homologue thereof; or (c) a DNMT3L domain or homologue thereof (i.e. the ATR may be as described herein). The separate effector protein that is not operably linked to a DNA-binding domain may comprise a KRAB, DNMT3A, DNMT3B, DNMT1 or DNMT3L domain or homologue thereof. The separate effector protein may be an effector domain/protein as described herein. The separate effector protein may be a full-length protein or functional fragment thereof. Preferably the separate effector protein is different to the effector domain of the ATR. Preferably the separate effector protein is of a different class to the effector domain of the ATR. Preferably the separate effector protein is selected such that it does not comprise a domain belonging to the same group (a), (b) or (c) as the effector domain that constitutes the ATR.

The ATR may also comprise a DNA-binding domain operably linked to a SETDB1 effector domain or homologue thereof. The separate effector protein that is not operably linked to a DNA-binding domain may also comprise a SETDB1 domain or homologue thereof.

In another aspect, the present invention provides a method of silencing a target gene comprising the step of administering the ATR of the invention, or the ATR and separate effector protein of the invention, or polynucleotides encoding therefor, to a cell. The method may be an in vitro method.

In addition, it is envisaged that the ATR or separate effector protein of the invention may comprise a SETDB1 domain or homologue thereof, when another component of the product of the invention (i.e. the ATR or separate effector protein) comprises a DNMT3A, DNMT3B or DNMT1 domain or homologue thereof.

The methods and uses of the present invention, for example methods of gene therapy or silencing a target gene, may also include a step of inactivating an endogenous gene that may counteract the activity of the ATRs or separate effector proteins of the invention. For example, the DNMT3B gene may be inactivated. The inactivation of this method step may, for example, be transient or permanent. The inactivation may, for example, be accomplished by genetic deletion, for example by using CRISPR/Cas9-based approaches, or by post-transcriptional downregulation, for example by using sh/siRNAs, or by transcriptional downregulation, for example by using an individual KRAB-based ATR targeted to the regulatory sequences of the gene of interest. Inactivating DNMT3B may be particularly preferred when three ATRs individually comprising KRAB, DNMT3A and DNMT3L domains are used.

### DESCRIPTION OF THE DRAWINGS

FIG. 1

Schematic detailing the experimental cell model.

An eGFP expression cassette (based on the hPGK promoter) followed by the TetO7 sequence is integrated within the first intron of the PPP1R12C gene (also known as the AAVS1 locus) of K562 cell line. Single cell derived clones containing homozygous insertion of the cassette are then transduced with a vector expressing ATRs (with candidate Repressive—Rep.—domains) and, after deposition of repressive epigenetic marks (red lollipops), the cells are treated or not with doxycycline. Maintenance of silencing or reactivation of eGFP expression is then evaluated by measuring eGFP expression.

FIGS. 2A-2F

Comparison of epigenetic silencing induced by tetR:K and tetR:D3A.

FIG. 2A. Schematics of the Bidirectional Lentiviral Vectors (Bid.LVs) expressing tetR:K and the marker gene mOrange (on the left) or tetR:DNMT3A and the marker gene ALNGFR (on the right). FIG. 2B. TetO7.eGFP reporter clones were transduced with the Bid.LV-tetR:K or with the Bid.LV-tetR:D3A in presence or in absence of doxycycline (right and left graphs, respectively), and analysed by flow cytometry over time to measure the percentage of eGFP-negative cells. FIGS. 2C-2D. Representative dot plot analysis at the indicated time-points of TetO7.eGFP reporter cell line transduced with Bid.LV-tetR:K or Bid.LV-tetR:D3A in presence or absence of doxycycline. The Mean Fluorescence Intensity (MFI) of eGFP silenced cells is compared to the MFI of untreated, wild-type cells. FIG. 2E. Silenced cells from the minus doxycycline conditions in (FIG. 2B) were sorted to purity and kept in culture with or without doxycycline to assess of eGFP reactivation. Representative dot plots of the cells in presence or absence of the drug are shown at the bottom. FIG. 2F. The silenced sorted cells from the conditions in (FIG. 2B) were treated for 7 days with AZA (1 μM) or vehicle (DMSO) and analysed for expression of eGFP (histogram on the left). Representative dot plots of vehicle and AZA treated cells are shown.

FIGS. 3A-3B

TetR:D3A-induced transcriptional repression is confined to the target locus.

FIG. 3A. Schematic of the AAVS1 locus. The genes surrounding the reporter cassette (red arrow) are indicated. FIG. 3B. Histograms showing the fold changes in the expression levels of the indicated genes between eGFP-negative cells silenced with either the Bid.LV-tetR:K or the Bid.LV-tetR:D3A, and untreated cells. The relative expression level of each gene was normalised to the expression of B2M, and represented as fold change relative to the untreated cells (calibrator) (n=3).

FIGS. 4A-4E

Synergistic activity of tetR:K and tetR:D3A upon their transient co-delivery.

FIG. 4A. The TetO7.eGFP reporter cell line was transiently transfected with plasmids encoding for tetR:K and tetD3A, either alone or in combination. The cells were analysed by flow cytometry over time and efficiency of silencing was measured as percentage of eGFP negative cells after transfection. Representative dot plot for each transfection condition are shown at the bottom of the histogram (n=3). FIG. 4B. Histogram showing the fold changes in the expression levels of the indicated genes between sorted eGFP-silenced cells from the mixed conditions shown in (FIG. 4A) and untreated cells. The relative expression level of each gene was normalised to B2M, and represented as fold change relative to the untreated cells (calibrator) (n=3). FIG. 4C. The eGFP-negative cells from the mixed-treated condition in (FIG. 4A) were sorted and then treated with AZA or DMSO (histogram showing the percentage of eGFP positive cells after 7 day from the indicated treatments; n=3). FIG. 4D. Similar experiment as in (FIG. 4A) but performed with in vitro transcribed mRNA encoding for tetR:K and tetD3A, delivered either alone or in combination. FIG. 4E. Histogram showing the fold changes in expression levels of the indicated genes between sorted eGFP-silenced cells from the mixed conditions shown in (FIG. 4D) and untreated cells (n=3).

FIGS. 5A-5D

Gene silencing with the tetR:K and tetR:D3A combination is locus and cell-type independent.

FIG. 5A. Schematic of the TetO7 reporter LV used in the study. TetO7 sequence was cloned upstream the hPGK promoter driving the expression of the eGFP reporter transgene. FIG. 5B. Graph showing the kinetics of eGFP silencing (% of eGFP-negative cells by flow cytometry) in the K562 LV/TETO7 reporter cell line transfected with in vitro transcribed mRNA encoding for tetR:K and tetR:D3A, delivered either alone or in combination (n=3; data are represented as mean±S.E.M.). FIGS. 5C-5D. Graphs showing the kinetics of eGFP silencing (% of eGFP-negative cells by flow cytometry) in the U937LV/TETO7 cell line (FIG. 5C) or in the B-lymphoblastoid LV/TETO7 reporter cell line (FIG. 5D). Cells were transfected as indicated in (FIG. 5B) (n=1 for U937 and n=3 for B-lymphoblastoid cells).

FIGS. 6A-6C

Screening of additional epigenetic effector domains for ATRs.

FIG. 6A. Graph showing the kinetics of eGFP silencing (% of eGFP-negative cells by flow cytometry) in the K562 LV/TETO7 reporter cell line upon transduction with lentiviral vectors expressing the indicated tetR-based ATRs (n=3). FIG. 6B. Graph showing the percentage of cells positive for the indicated LVs over time in culture (n=3). FIG. 6C. Graph showing the kinetics of eGFP silencing (% of eGFP-negative cells by flow cytometry) in the K562 LV/TETO7 reporter cell line transduced with lentiviral vectors stably expressing the indicated tetR-based ATRs, before and after doxycycline administration (n=3).

FIGS. 7A-7H

Screening of additional combinations of artificial transcription repressors (ATRs) in different mammalian cells.

FIGS. 7A-7D. Graphs showing the kinetics of eGFP silencing (% of eGFP-negative cells by flow cytometry) in the K562 LV/TETO7 reporter cell line upon electroporation with individual plasmids encoding for the indicated tetR-based ATRs (FIG. 7A; n=3; data are represented as mean±S.E.M.), or upon electroporation with the plasmid encoding for tetR:D3A plus the plasmid encoding for one of the other tetR-based ATRs (FIG. 7B; n=3; data are represented as mean±S.E.M.), or upon electroporation with the plasmid encoding for tetR:K plus the plasmid encoding for one of the other tetR-based ATRs (FIG. 7C; n=3; data are represented as mean±S.E.M.), or upon electroporation with the plasmids encoding for tetR:D3A+tetR:K in conjunction with the plasmid encoding for one of the other tetR-based ATRs (FIG. 7D; n=3; data are represented as mean±S.E.M.). FIG. 7E. Histogram showing the efficacy of eGFP silencing 21 days post electroporation of the K562 LV/TETO7 reporter cell line with plasmids encoding for the indicated tetR-based ATRs (later time-points of A-D; n=3; data are represented as mean±S.E.M.). FIG. 7F. Histogram showing the efficiency eGFP silencing 30 days post electroporation of the K562 LV/TETO7 reporter cell line with plasmids encoding for the indicated tetR-based ATRs, including that based on SETDB1 (n=3; data are represented as mean±S.E.M.). FIG. 7G. Graph showing the kinetics of eGFP silencing of the B-lymphoblastoid LV/TETO7 reporter cell line electroporated with mRNA encoding for the indicated tetR-based ATRs (n=3; data are represented as mean±S.E.M.). FIG. 7H. Graph showing the kinetics of eGFP silencing of the murine NIH/3T3 LV/TETO7 reporter cell line electroporated with mRNA encoding for the indicated tetR-based ATRs (n=2; data are represented as mean±range).

FIGS. 8A-8C

Gene silencing by transient co-delivery of artificial transcription repressors (ATRs) comprising custom-made DNA-binding domains (head-to-tail orientation).

FIG. 8A. Schematic representation of the various Artificial Tale Binding Sites.(head-to-tail).hPGK.eGFP cassettes semi-randomly integrated in the genome of K562 cells via LV transduction, differing in spacer length between the two ATRs binding sites. Two different TALE domains have been separately fused to each epigenetic effector, thus leading to two alternative co-delivery strategies differing for the D3A-K relative order of binding on the target. FIG. 8B. Graphs showing the silencing efficiency (% of eGFP-negative cells at 34 days post-electroporation) with respect to the spacer length, in the K→D3A (Left) and D3A→K (Right) relative ATRs order of binding on the target. FIG. 8C. Graphs showing the kinetics of eGFP silencing in the cell line with the 25 bp spacer (the best-performing spacer tested in the experiments shown in FIG. 8B) in the K→D3A (Left) and D3A→K (Right) relative ATRs order of binding on the target (n=3; data are represented as mean±S.E.M.).

FIGS. 9A-9C

Gene silencing by transient co-delivery of artificial transcription repressors (ATRs) comprising custom-made DNA-binding domains (head-to-head orientation).

FIG. 9A. Schematic representation of the various Artificial Tale Binding Sites.(head-to-head).hPGK.eGFP cassettes semi-randomly integrated in the genome via LV transduction, differing in the spacer length between the two ATRs binding sites. Two different TALE domains have been separately fused to each epigenetic effector, thus leading to two alternative co-delivery strategies differing for the D3A-K relative order of binding on the target. FIG. 9B. Graphs showing silencing efficiency (i.e. % of eGFP-negative cells at 34 days post-electroporation) with respect to the spacer length, in the D3A→K (Left) and K→D3A (Right) relative ATRs order of binding on the target. FIG. 9C. Graphs showing the kinetics of eGFP silencing in the cell line with the 15 bp spacer (the best-performing spacer tested in the experiments shown in B) in the D3A→K (Left) and K→D3A (Right) relative ATRs order of binding on the target (n=3; data are represented as mean±S.E.M.).

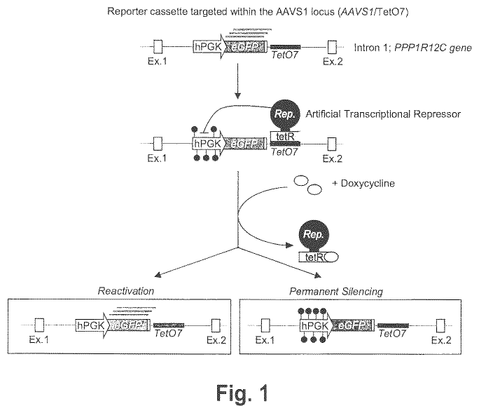

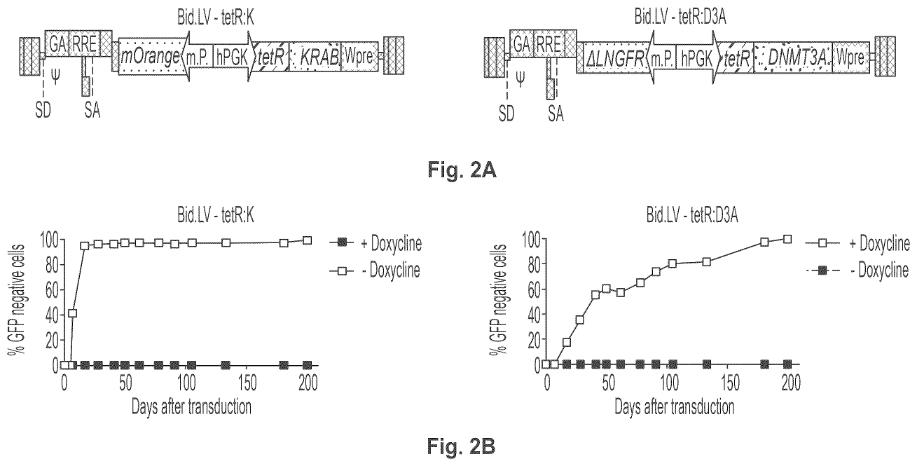

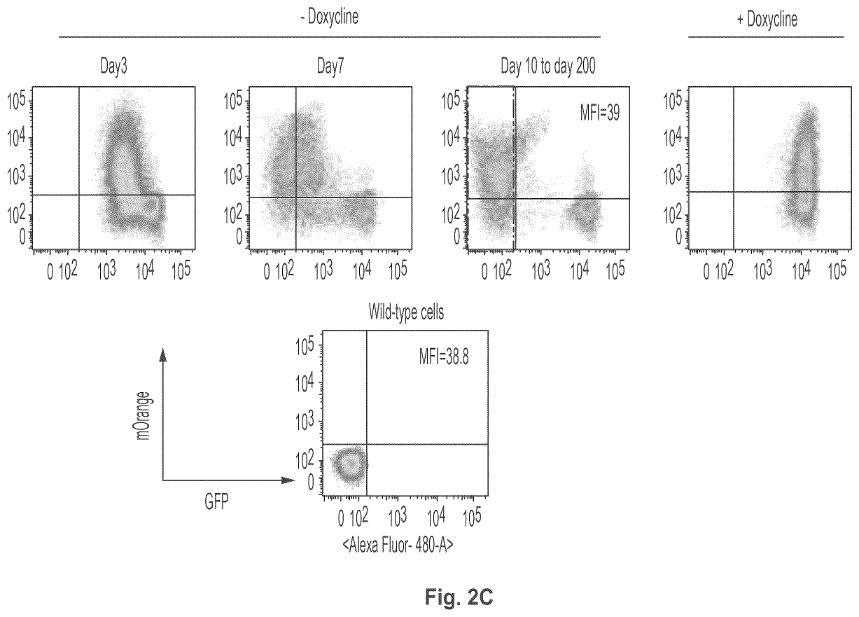

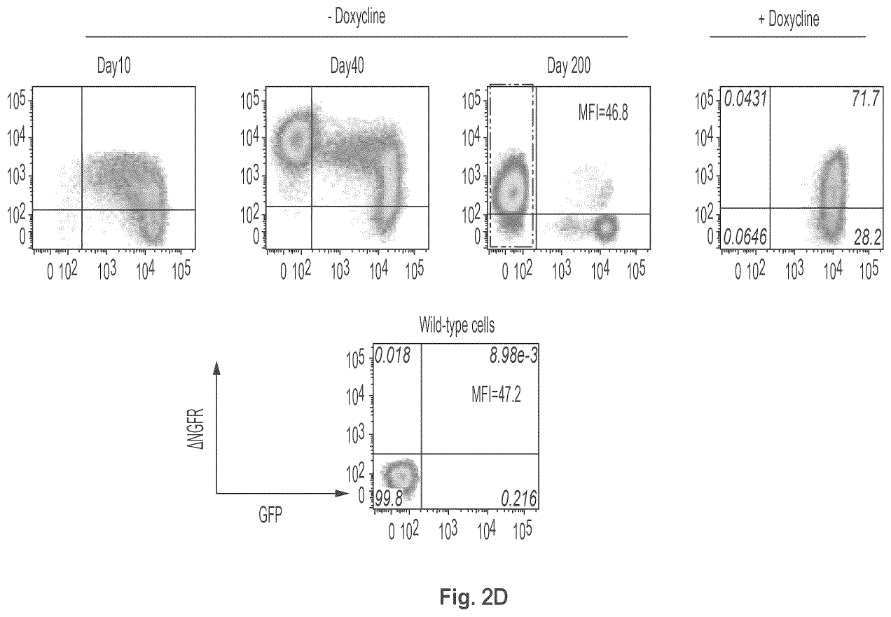

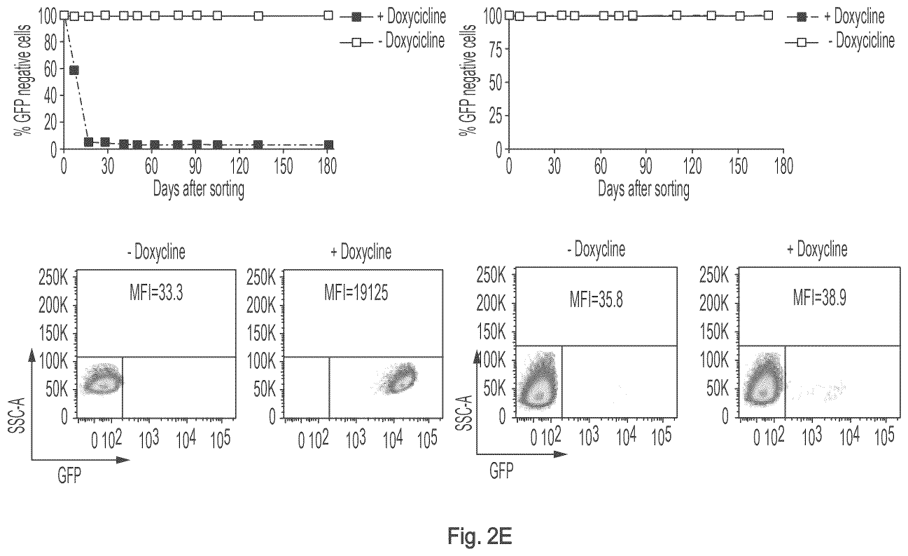

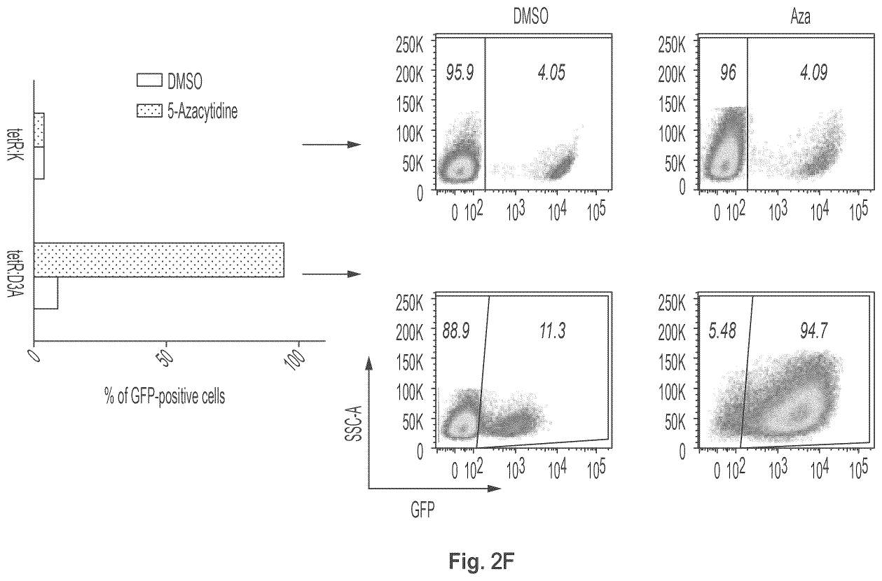

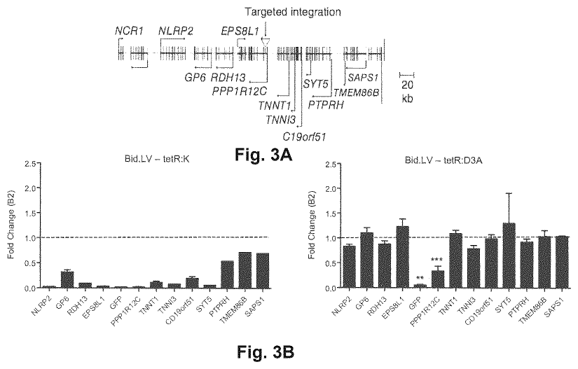

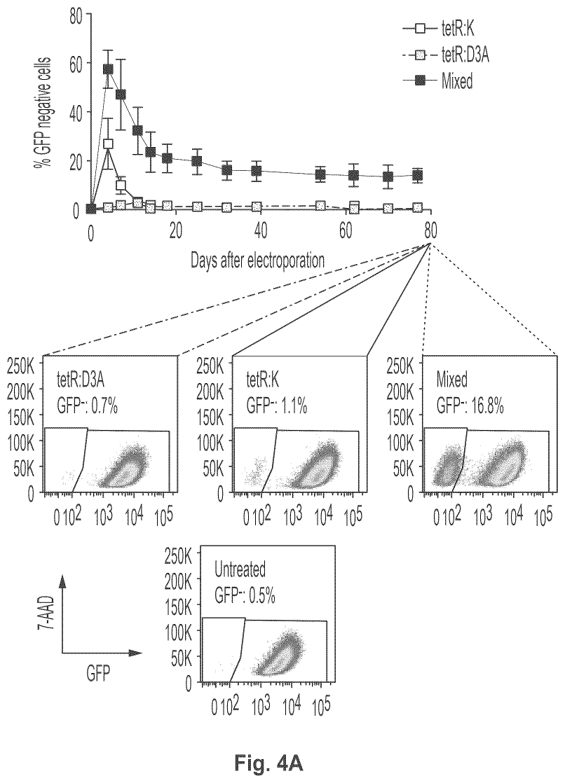

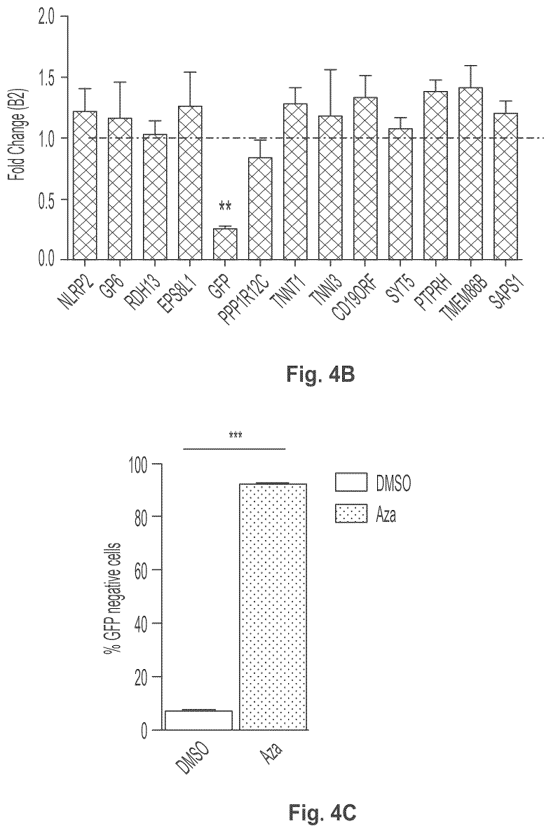

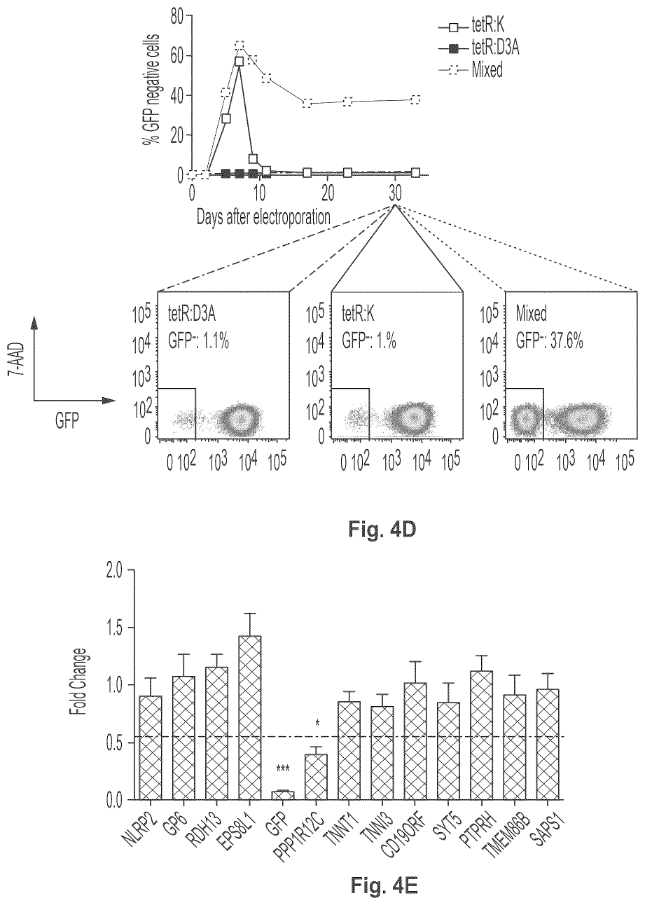

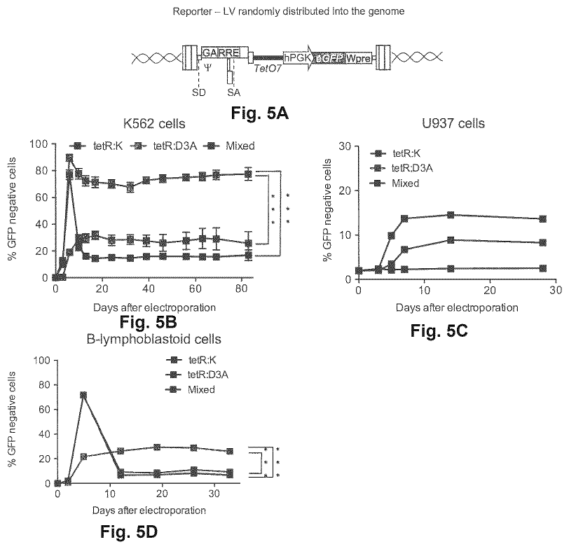

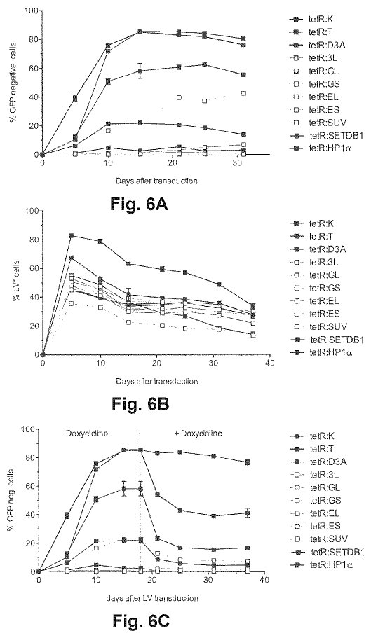

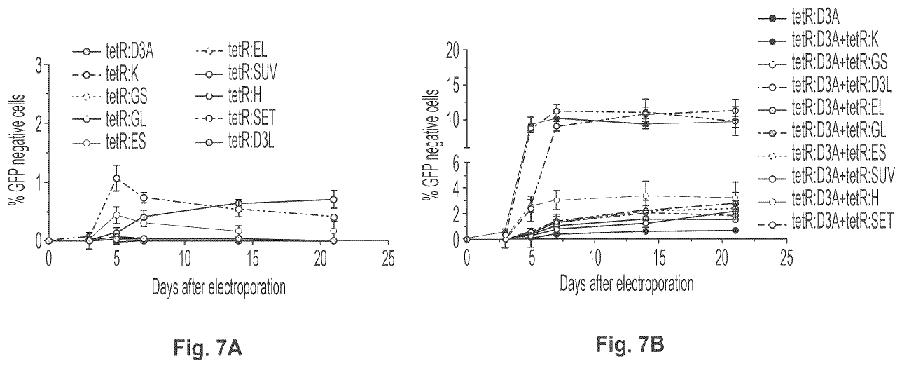

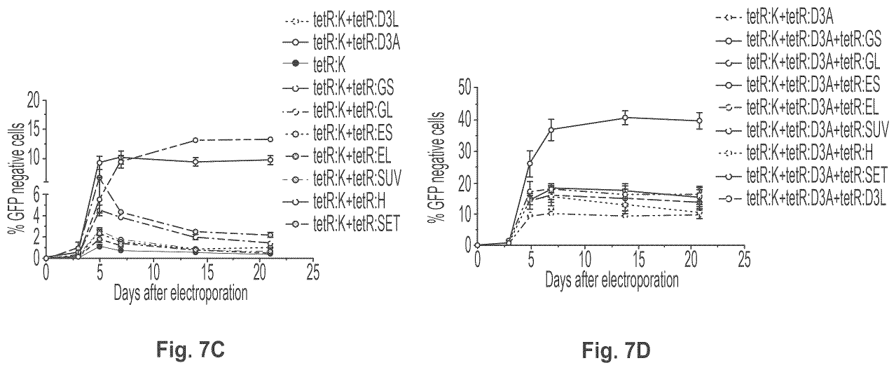

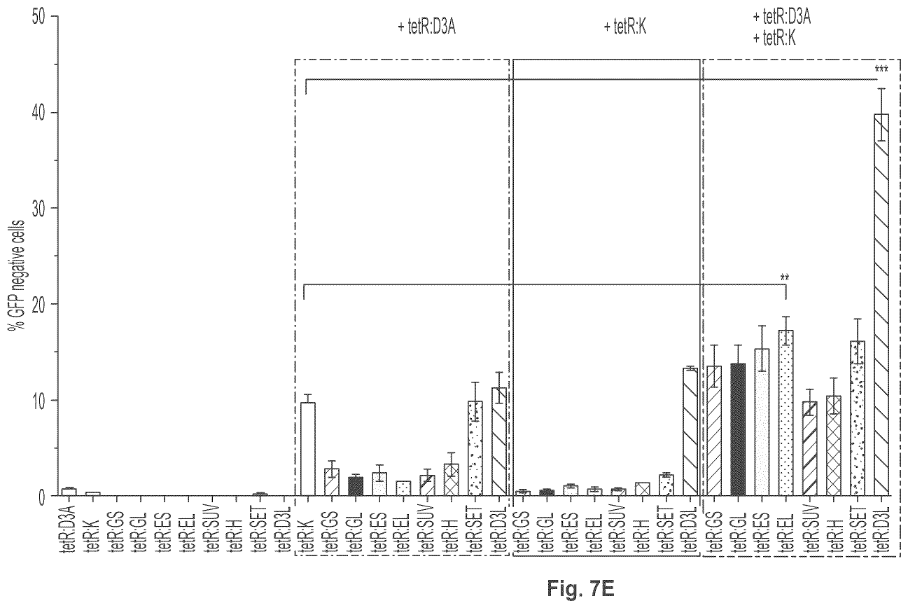

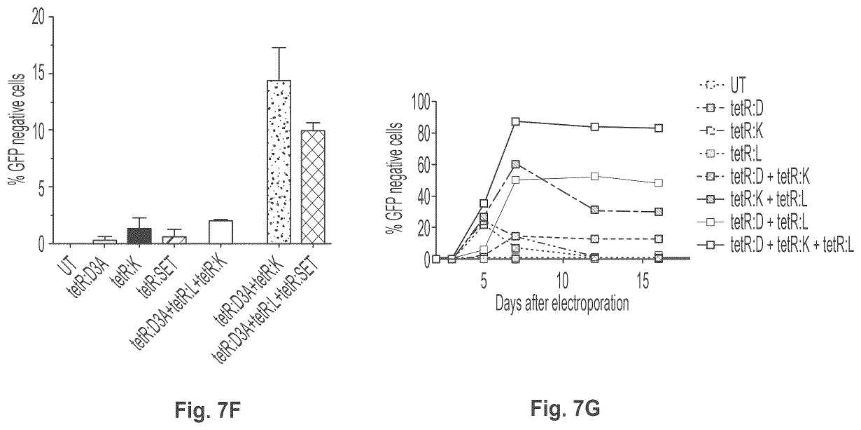

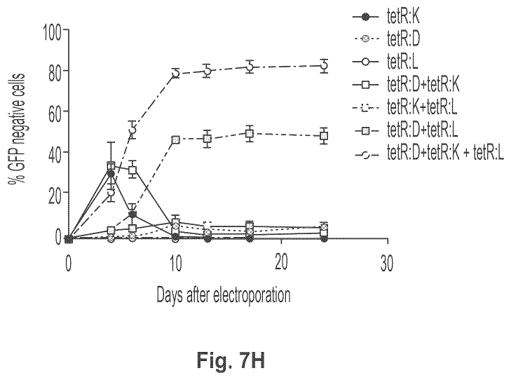

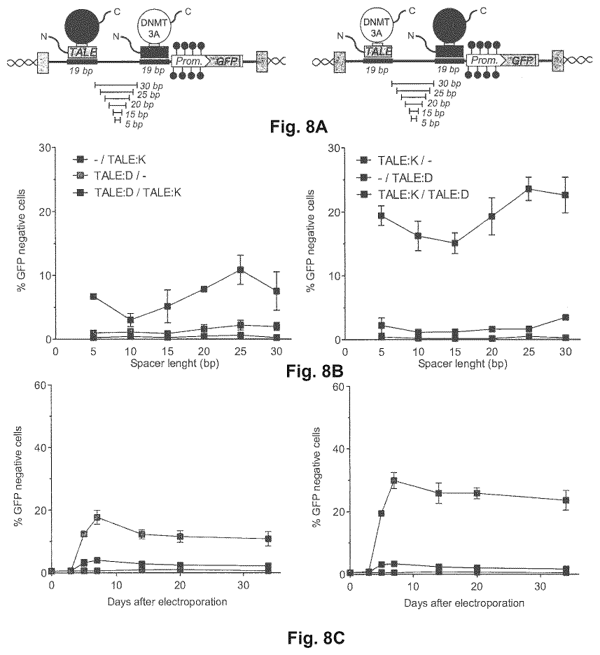

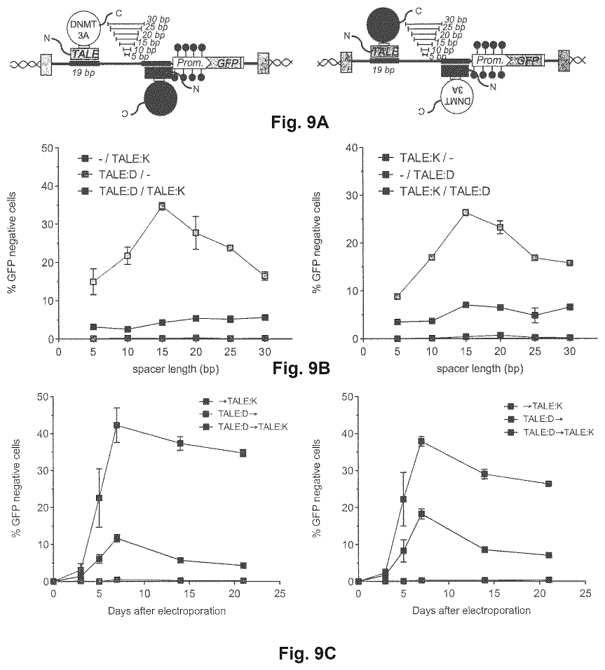

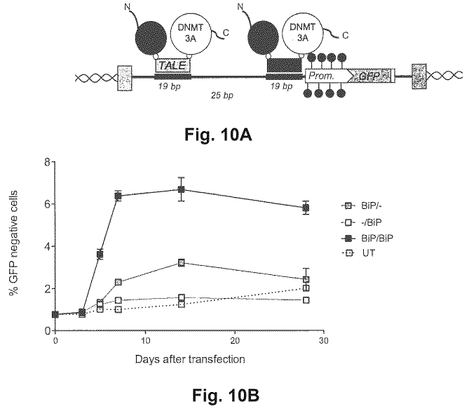

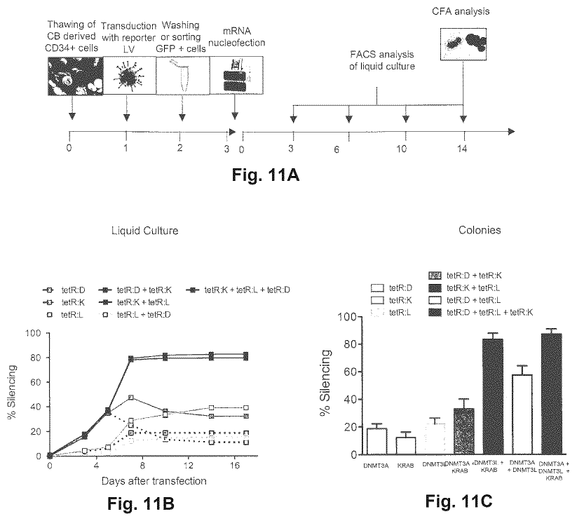

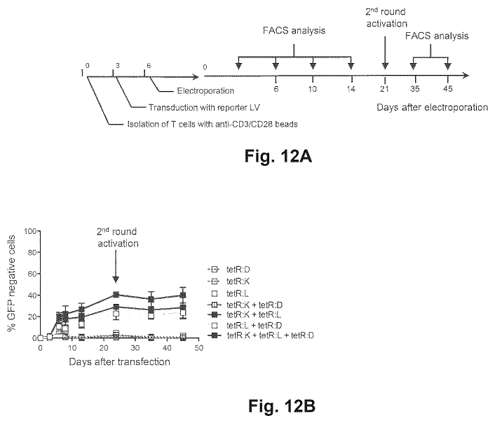

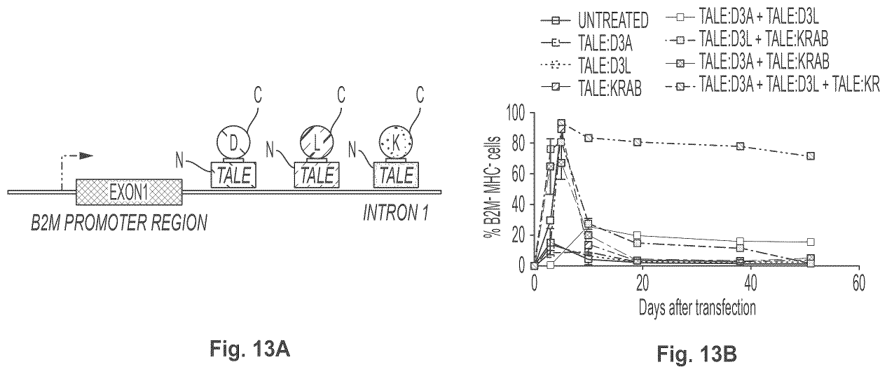

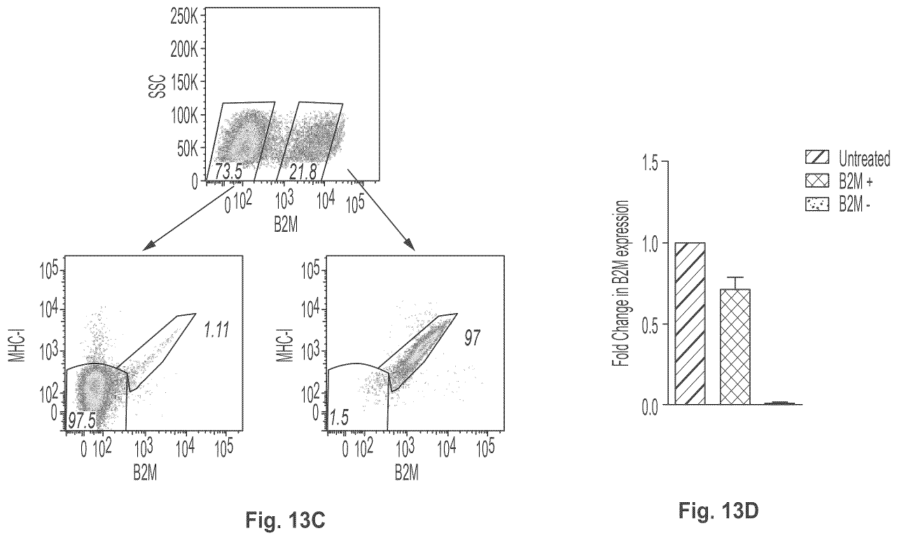

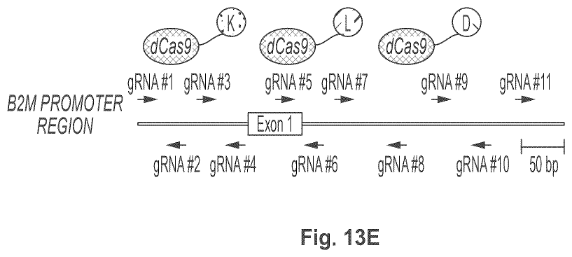

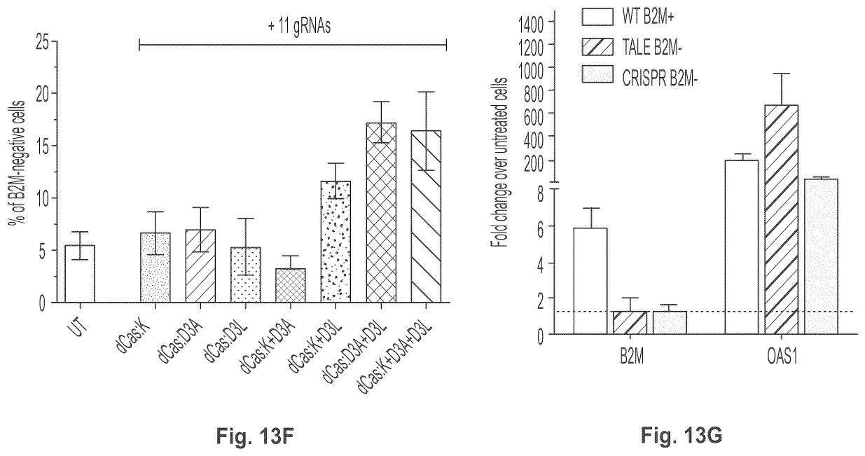

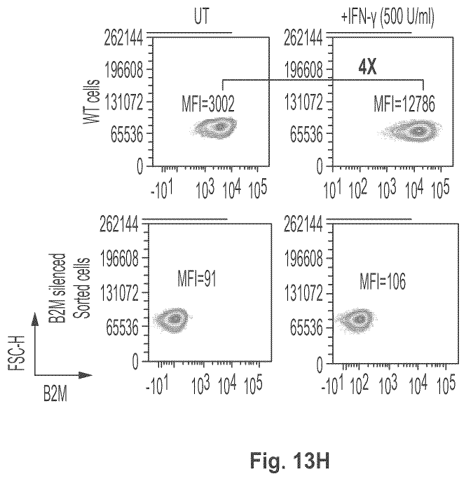

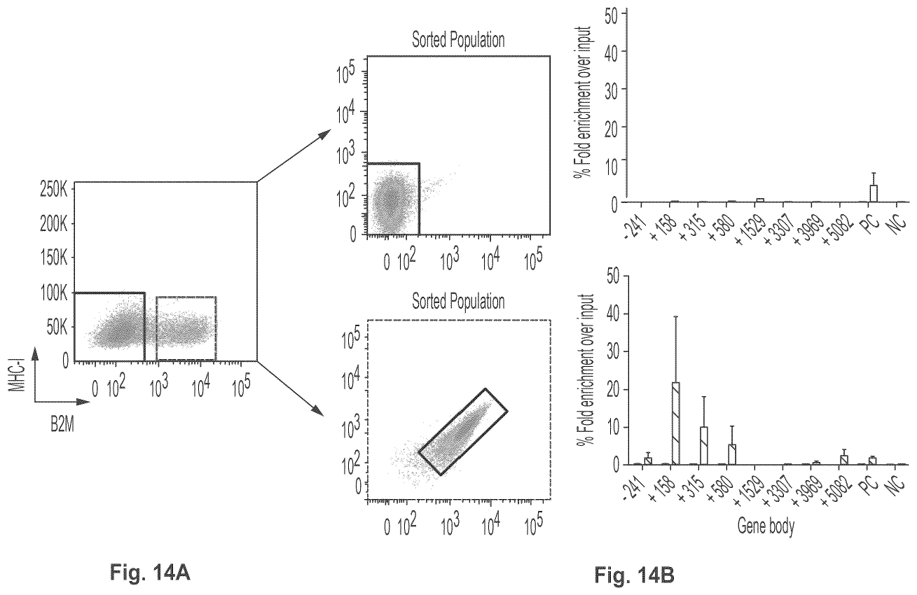

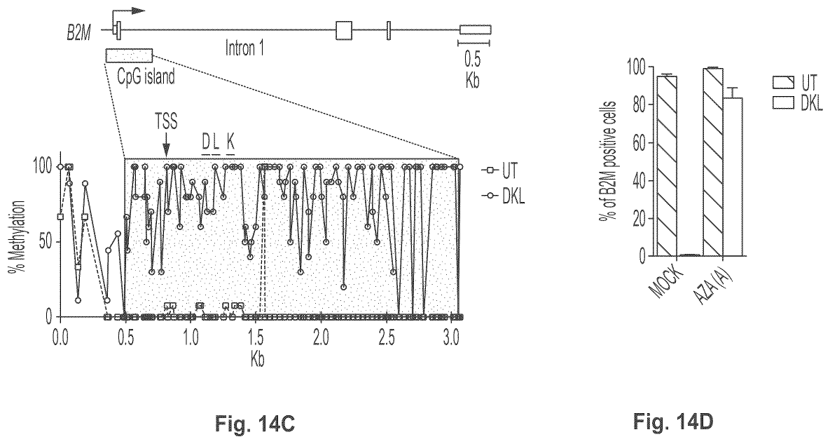

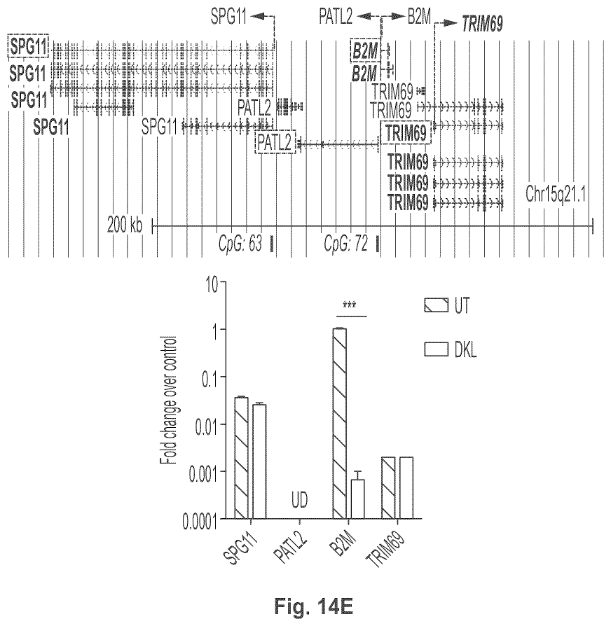

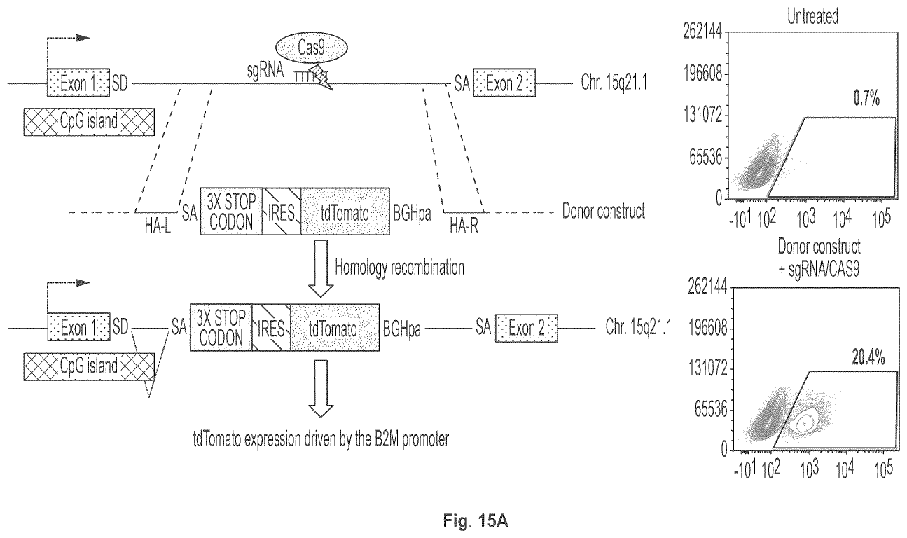

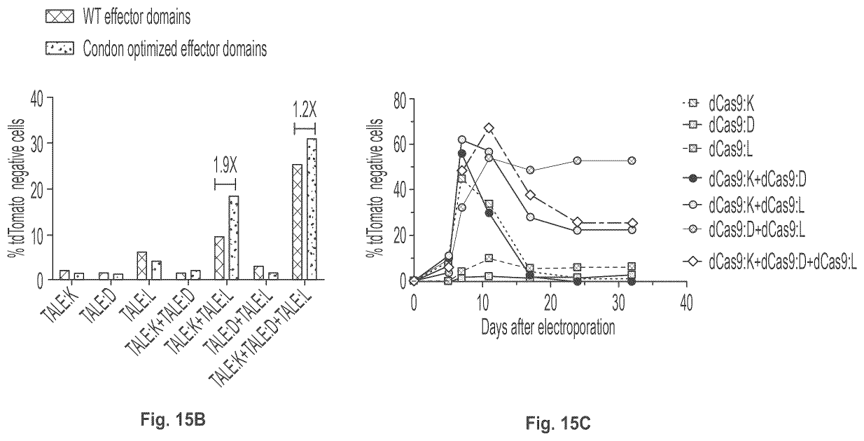

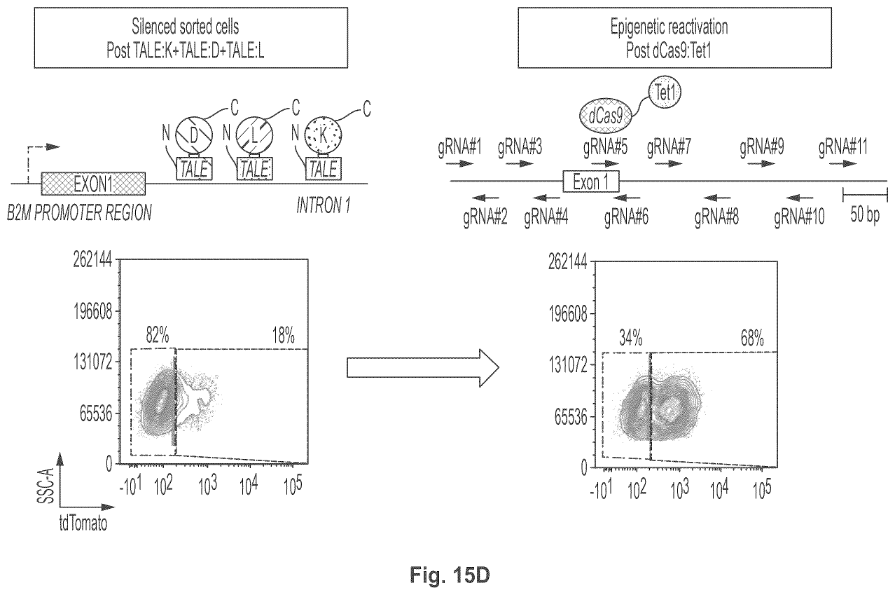

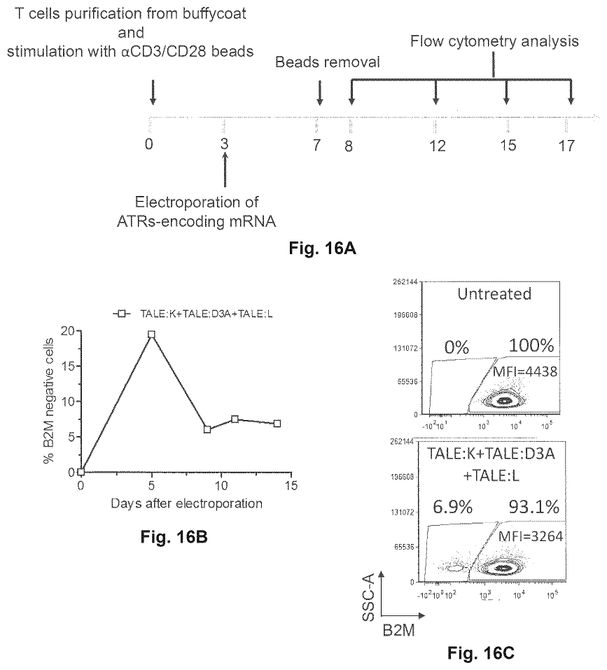

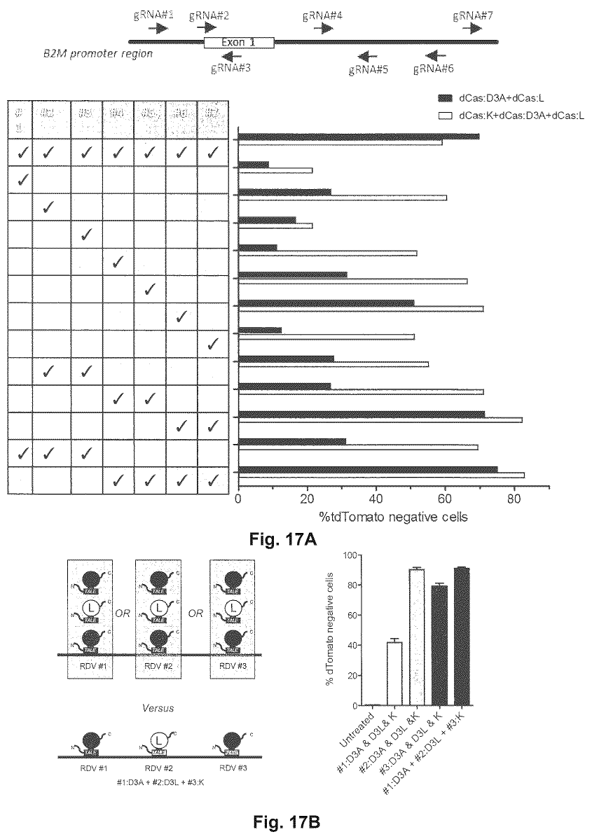

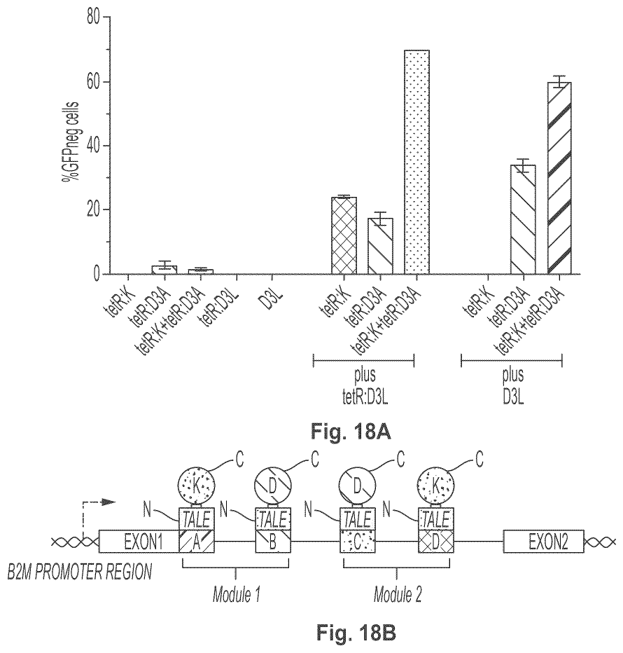

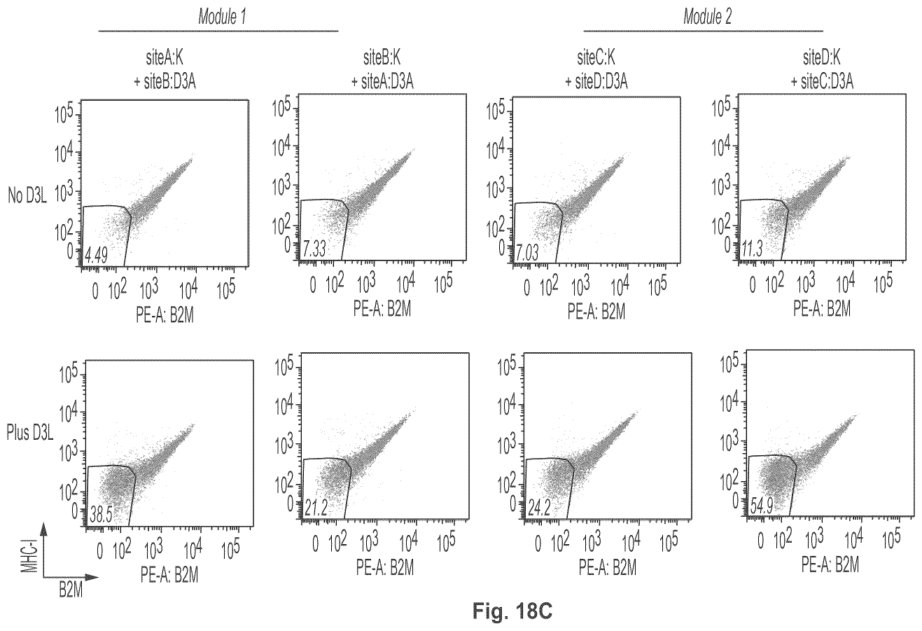

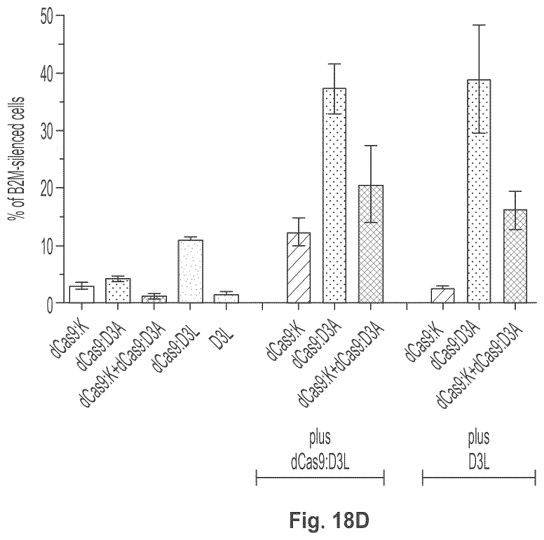

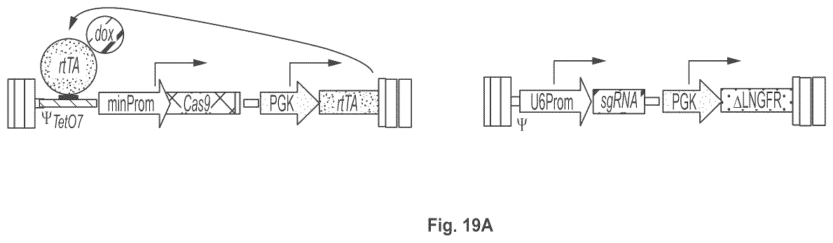

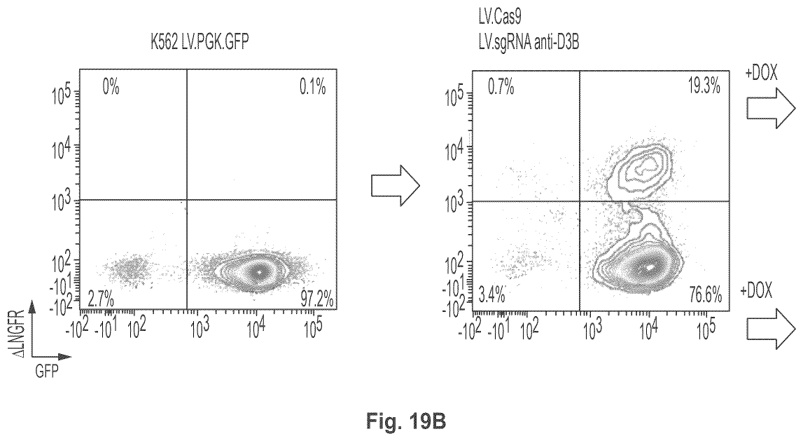

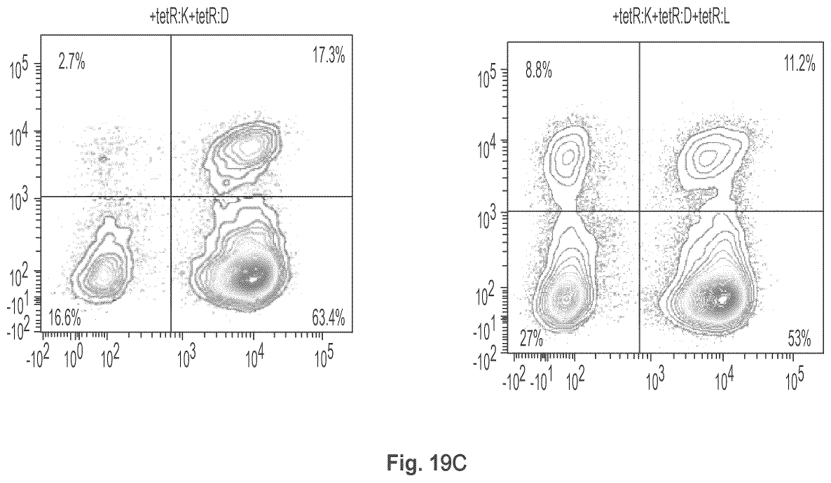

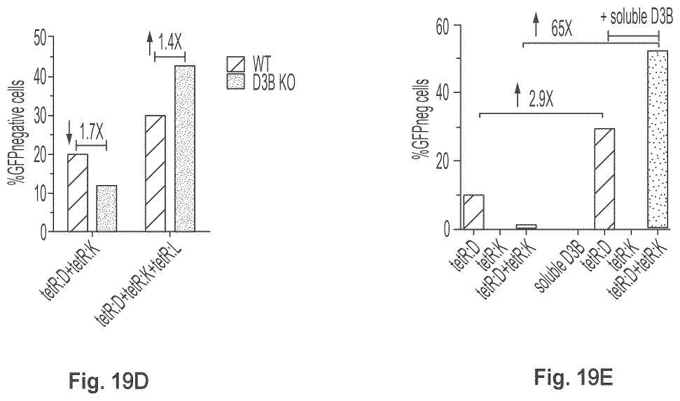

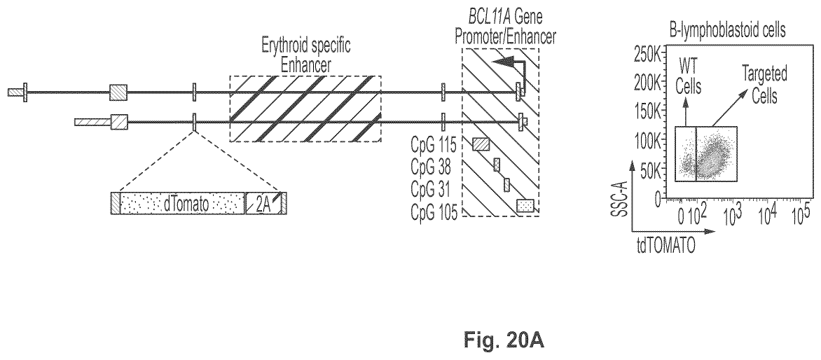

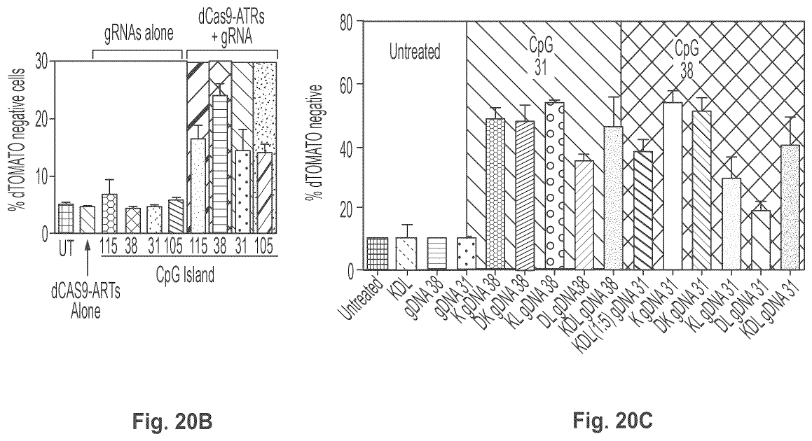

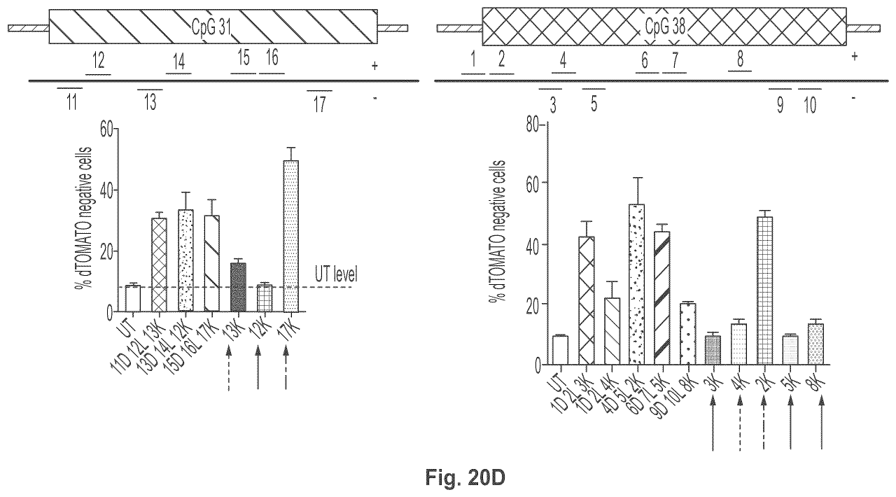

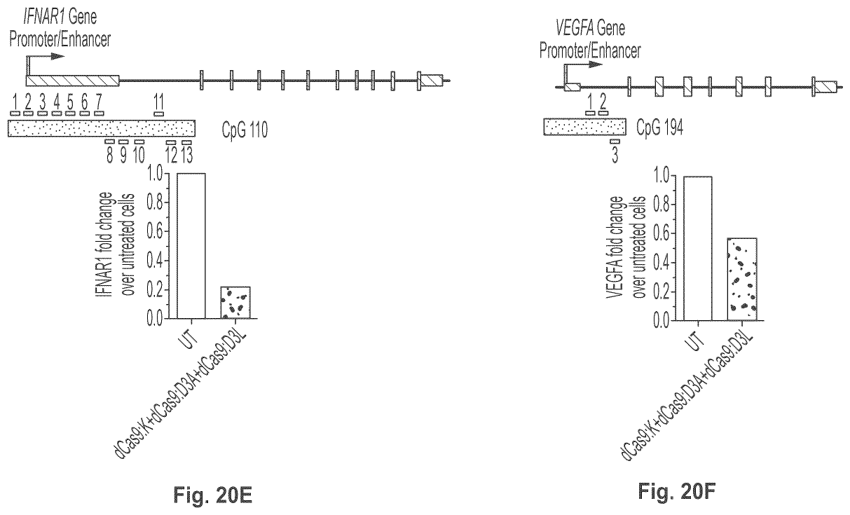

FIGS. 1A-10B

Gene silencing with artificial transcription repressors (ATRs) comprising more than one effector domain.

FIG. 10A. Schematic representation of the Artificial Tale Binding Sites (head-to-head).hPGK.eGFP cassette semi-randomly integrated in the genome of K562 cells via LV transduction bound by chimeric K:tetR:D3A ATR (Bi-Partite; BiP), where KRAB and DNMT3A domains were fused to the N- and C-terminus (respectively) of the same DNA binding domain. FIG. 10B. Graphs showing the kinetics of eGFP silencing in the cell line with the 25 bp spacer (the same line used in of FIG. 8B), transfected with plasmids encoding for the Bi-Partite (BiP) fusion protein, when transfected alone or in combination (n=3; data are represented as mean±S.E.M.).

FIGS. 11A-11C

Permanent epigenetic silencing in human haematopoietic stem and progenitor cells (HSPCs) by using different combinations of artificial transcription repressors (ATRs).

FIG. 11A. Schematic time-line of the protocol used to assess efficiency of silencing in HSPCs.

Briefly, on day 0, the human CD34+ cells were thawed in stimulating media with early acting cytokines and transduced at day 1 with the TetO7 reporter LV (schematic of the vector in FIG. 5A). Cells were then washed and electroporated with in vitro transcribed mRNA on day 3 from thawing. The day after, 800 cells were plated for CFC-U assays, while the remaining cells were grown in liquid culture and analysed by flow cytometry at the indicated time points. CFC-U analysis was performed 14 days after thawing. FIG. 11B. Graph showing the kinetic of silencing of eGFP in liquid cultured human CD34⁺ transfected with in vitro transcribed mRNA encoding for the indicated ATRs, delivered either alone, or in double, or triple combinations (data were normalised to the un-electroporated but LV-transduced control; n=3; data are represented as mean±S.E.M.). FIG. 11C. Histogram showing the percentage of eGFP-silencing in erythroid and myeloid colonies derived from the human CD34+ transfected with in vitro transcribed mRNAs as indicated in (FIG. 11B) (n=3; data are represented as mean±S.E.M.).

FIGS. 12A-12B

Permanent epigenetic silencing in human T lymphocytes by using different combinations of artificial transcription repressors (ATRs).

FIG. 12A. Schematic time-line of the protocol used in this study to assess efficiency of silencing in human primary T cells. Briefly, on day 0, T-cells were isolated with anti-CD3/CD28 coated beads and left in culture 3 days before transduction with the reporter TetO.LV. On day 6, the cells were transfected with in vitro transcribed mRNA encoding for the indicated ATRs, and expression of eGFP was measured by flow cytometry at the indicated time points. At 3 weeks post-transfection, cells were re-stimulated and stability of eGFP silencing was measured by flow cytometry. FIG. 12B. Graph showing the kinetic of eGFP-silencing in human primary T cells transfected with in vitro transcribed mRNA encoding for the indicated ATRs, which were delivered either alone, or in double, or triple combinations normalised over untreated cells (data were normalised to the un-electroporated but LV-transduced control; n=2; data are represented as mean±range).

FIGS. 13A-13H

Permanent epigenetic silencing of the human β2-microglobulin (B2M) gene using artificial transcription repressor (ATR) combinations.

FIG. 13A. Schematic representation of the B2M locus indicating the binding sites of the TALE-based ATRs. FIG. 13B. Graph showing the kinetics of B2M silencing in HEK-293T cells electroporated with plasmids encoding for the indicated TALE-based ATRs, which were delivered either alone or in combination (n=3; data are represented as mean±S.E.M.). FIG. 13C. Representative flow cytometry dot plots of HEK-293T cells transfected with plasmids encoding for the triple TALE:ATR combination (plot on the top), and the cell sorting strategy used to enrich for the double negative (bottom plot on the left) and the double positive (bottom plot on the right) cells. FIG. 13D. Histogram showing the fold change in the expression levels of the B2M gene in the sorted cells from (FIG. 13C) and in untreated HEK293T cells (n=3; data are represented as mean±S.E.M.). FIG. 13E. Schematics of the dCas9-based ATRs and of the gRNAs selected to target the CpG island located in the B2M promoter region. FIG. 13F. Histogram showing the silencing efficiency at day 33 post-CRISPR/dCas9-based ATRs plasmid electroporation (n=3; data are represented as mean±S.E.M.). FIG. 13G. The B2M silenced cells from FIGS. 2C-2D (named TALE B2M- in this panel), the B2M-silenced cells sorted from the triple-CRISPR/dCas9 based ATR combination in FIG. 2F (named TALE B2M- in this panel), and wild-type HeK-293T cells (named WT B2M+ in this panel) were exposed or not to IFN-γ, and then analysed to measure the expression levels of B2M and OAS1. Histogram showing the fold change in the expression levels of the B2M and the OAS1 gene between IFN-γ and untreated cells. The expression of the Hypoxanthine Phosphoribosyltansferase 1 (HPRT1) gene was used as normaliser (n=3; data are represented as mean±S.E.M.). FIG. 13H. Representative flow cytometry dot plots of the indicated HEK-293T populations either untreated (plots on the left) or at 4 days post IFN-γ treatment (plots on the right). Numbers indicate the MFI B2M.

FIGS. 14A-14E

Silencing of β2-microglobulin (B2M) is associated with significant epigenetic editing of the gene.

FIG. 14A. Representative flow cytometry dot plots of HEK-293T cells transfected with plasmids encoding for the triple TALE:ATR combination, and the cell sorting strategy used to enrich for the double positive and double negative cells. FIG. 14B. ChIP analysis performed on untreated (top histogram) and silenced cells from (FIG. 14A) (bottom histogram) for the presence of the RNA PollI. Histogram shows the fold enrichment in RNA PollI over the input in relation to the distance of the qPCRs assays from the Transcription Start Site (TSS; set at +1) of the gene (n=3; data are represented as mean±S.E.M.). The ubiquitously transcribed AAVS1 locus was used as Positive Control (PC) for RNA PollI enrichment, while the silent CCR5 gene as a Negative Control (NC). FIG. 14C. Bisulfite analysis of the B2M CpG island in untreated (UT) and silenced cells. The TSS of the gene and relative position of binding site of the three TALE:ATRs (D; L;K) are indicated. FIG. 14D. Histogram showing the percentage of B2M positive cells at day 7 upon AZA treatment (n=3; data are represented as mean±S.E.M.). FIG. 14E. Top: schematic representation of the B2M locus. The CpG islands within this locus are depicted in green. Bottom: histogram showing the fold change in gene expression levels of the indicated genes between silenced and untreated cells. Genes with a Ct value ≥37 were excluded from the analysis. The relative expression level of each gene was normalized to HPRT, and represented as fold change relative to the untreated cells (calibrator).

FIGS. 15A-15D

Silencing of β2-microglobulin (B2M) is effective in another human cell line.

FIG. 15A. Schematics (on the left) of the CRISPR/Cas9-based gene targeting strategy used to insert the tdTomato transgene under the control of the B2M promoter. Representative flow cytometry dot plots of K-562 cells pre- and post-gene targeting (upper and bottom right, respectively). FIG. 15B. Histogram showing the B2M silencing efficiency (i.e. dtTomato-negative cells) at day 30 post electroporation with plasmids encoding for the indicated TALE-based ATRs carrying either the wild-type (WT) or the codon-optimised effector domains (n=1). FIG. 15C. Graph showing the kinetics of B2M silencing (measured as % of dtTomato-negative cells) of K-562 cells electroporated with plasmids encoding for the indicated CRISPR/dCas9-based ATRs (n=1). FIG. 15D. Representative flow cytometry analyses of: (i) sorted tdTomato-negative cells post-transfection with in vitro transcribed mRNAs encoding for the triple TALE:ATR combination (left schematic and dot plot); (ii) the cells from (i) upon transfection with a plasmid encoding for the dCas9:Tet1 in conjunction with plasmids for the B2M gRNAs (left schematic and dot plot) (n=1).

FIGS. 16A-16C

Silencing of β2-microglobulin (B2M) is effective in primary T-lymphocytes.

FIG. 16A. Schematics of the experimental workflow. FIG. 16B. Graph showing the kinetics of B2M silencing in human T-lymphocytes electroporated with mRNAs encoding for the triple TALE-based ATRs (n=1). FIG. 16C. Representative flow cytometry dot plots of the indicated T-lymphocytes populations 14 days post-treatment. The percentage of cells within the indicated gates and the B2M MFI are shown.

FIGS. 17A-17B

Single ATR binding site is sufficient for effective silencing of the endogenous gene both with Cas9 and TALE-based ATRs.

FIG. 17A. Top: schematic of the B2M gene indicating the relative location of the gRNAs (read arrows) selected to target the CpG island of this gene. Bottom: histogram showing the efficiency of B2M silencing (calculated as % of tdTomato-negative cells) 18 days post CRISPR/dCas9-based ATRs plasmid electroporation in the K562 B2M_tdTomato reporter cell line (n=1). FIG. 17B. Left: schematic of binding of TALE based-ATRs on the DNA. In the grey boxes are indicated the conditions in which each of the three different DNA binding domains (form #1 to #3; named in the FIG. 17B as Repeat Variable Diresidue—RDV) are equipped with both of the three different effector domains. Bottom to this schematic is depicted the condition in which the three different RDVs are equipped each with a different effector domain, as already shown in FIG. 13A. Right: histogram showing the percentage of tdTomato-negative cells upon transfection with plasmids encoding for the indicated TALE-based ATR combinations (n=3; data are represented as mean±S.E.M.).

FIGS. 18A-18D

Transient expression of an un-targeted DNMT3L improves and rescues silencing efficiency of the DNMT3A+KRAB based ATRs in refractory cell types.

FIG. 18A. Histogram showing the percentage of eGFP silencing of B-lymphoblastoid LV-TetO7 reporter cell line at 27 days post-transfection with in vitro transcribed mRNAs encoding for the indicated tetR-based ATRs, which were delivered in conjunction or not with either the tetR:D3L or with the un-targeted, full-length DNMT3L-encoding mRNA (n=2; data are represented as mean±range). FIG. 18B. Schematics of the B2M locus depicting the binding sites and the relative arrangement of the indicated TALE-based ATRs. Note that each Module can be bound by a pair of TALE-based ATRs. Moreover, for each Module, the relative order of the effector domain can be swapped. For example, for Module 1, site A can be bound by the KRAB-based ATR, while site B by DNMT3A-based ATR, or vice versa. FIG. 18C. Representative flow cytometry dot plots of B2M silencing in HEK-293T cells 21 days post-transfection with plasmids encoding for the indicated pairs of TALE-based ATRs (Module 1 or Module 2, shown for the two possible relative order of binding of the ATRs), which were delivered either alone (top plots row) or in combination with the un-targeted DNMT3L (bottom plots row). FIG. 18D. Histogram showing the percentage of B2M silencing of HEK-293T cells at 45 days post-transfection with plasmids encoding for the indicated dCas9-based ATRs and the cognate gRNAs (as those depicted in FIG. 13E), which were delivered either alone or in conjunction with the dCas9:D3L or with un-targeted, full-length DNMT3L-encoding plasmid (n=3; data are represented as mean±S.E.M.).

FIGS. 19A-19E

Genetic inactivation of the DNMT3B increases the silencing efficiency of the triple ATR combination in permissive cell lines, while transient expression of an un-targeted DNMT3B rescues silencing efficiency of the DNMT3A+KRAB combination in refractory cell types.

FIG. 19A. Schematics of the lentiviral vectors used to conditionally express Cas9 upon doxycycline administration (left) or to express the gRNA of interest (right). FIGS. 19B-19C. Representative flow cytometry analyses of: i) eGFP-positive K-562 cells upon transduction with the lentiviral vector described in FIG. 5A and then sorted to near purity for eGFP-expression (left plot); ii) the cell line from (i) upon were transduction with the LV encoding for the inducible Cas9 and with the LV encoding for the DNMT3B-gRNA (ALNGFR was used as a marker of transduction for the latter LV; middle plot). Note that this second cell line was then exposed to doxycycline for 7 days in order to activate the Cas9 expression and disrupt the coding sequence of the endogenous DNMT3B gene; iii) the cells from (ii) upon electroporation with plasmids encoding for either the double tetR:K+tetR:D3A (top right plot) or the triple tetR:K+tetR:D3A+tetR:D3L (bottom right plot) ATRs combinations. FIG. 19D. Histogram showing the percentage of eGFP silenced cells at day 19 post genetic disruption of the DNMT3B gene by the CRISPR/inducibleCas9 system (n=1). These numbers were obtained by calculating the silencing efficiencies in ALNGFR-positive (the cells with disruption of DNMT3B; red bars) and -negative cells (wild-type K-562 cells; blue bars). FIG. 19E. Histogram showing the silencing efficiency (% of eGFP-negative cells) in the B-lymphoblastoid TetO7 reporter cell line at day 27 post-transfection with mRNAs encoding for the indicated tetR-based ATRs, which were delivered in conjunction or not with the mRNA encoding for the un-targeted, wild-type DNMT3B sequence (data are shown as mean of the two experiments).

FIGS. 20A-20F

Permanent epigenetic silencing of additional human endogenous genes (using artificial transcription repressor (ATR) combinations.

FIG. 20A. Schematic (on the left) of the B-Cell Lymphoma/leukemia 11A (BCL 11A) gene showing the two transcript variants of this gene. Dashed boxes highlight gene regulatory elements. In particular, the gene promoter/enhancer region at the level of the transcription start site (yellow box) with cluster of 4 different CpG islands varying in size and number of CpG residues, and the erythroid specific enhancer (red box) responsible for the lineage restricted expression of the gene within the second intron of gene. In order to study both gene promoter and erythroid specific enhancer functions, the tdTomato transgene linked to the BCL11A transcript through a 2A self-catalytic peptide was targeted within the third exon of the gene. On the right, is shown a representative dot plot of B-lymphoblastoid cells after sorting of the tdTomato-positive cells. FIG. 20B. Histogram showing the percentage of tdTomato-negative cells at day 32 post-transfection with the indicated dCas9-based ATRs and the corresponding pools of gRNAs (namely, 11 gRNAs for CpG105; 8 gRNAs for CpG31; 9 gRNAs for CpG38; 10 gRNAs for CpG115) targeting the indicated CpG islands of BCL11A (n=3; data are represented as mean±S.E.M.). Untreated cells, or cells transfected with the pools of gRNAs alone or with the dCas9-based ATRs alone were used as controls. FIG. 20C. The tdTomato reporter cell line was co-transfected with plasmids encoding for dCas9-based ATRs, either alone, or in double or triple combination, and with plasmids for a pool of 9 gRNAs targeting the CpG 38, or with plasmids for a pool of 8 gRNAs for CpG 31. Silencing efficiency was measured at 2 weeks post-transfection and is reported in the histogram (n=3; data are represented as mean±S.E.M.). FIG. 20D. Top: Schematics of the binding sites of TALE-based ATRs targeting CpG 31 (top left) or CpG 31 (top right) of the BCL11A promoter region, and their relative orientation of binding on the DNA (+ indicates Watson strand, while—indicates Crick strand). Bottom: the dTomato reporter cell line was transfected with plasmids encoding TALE:KRAB alone, or with the indicated combinations of triple TALE-based ATRs, as labelled on the x axis of the histograms. Silencing efficiency is reported as percentage of dTomato-negative cells. Analysis was performed at day 18 post-plasmid transfection (n=3; data are represented as mean±S.E.M.). FIG. 20E. Top: Schematic of the Interferon (alpha, beta and omega) Receptor 1 (IFNAR1) gene. The green box highlights a CpG island (the number of CpG residues are indicated) at the level of the gene promoter/enhancer region. Bottom: Histogram showing the fold change in the expression level of the IFNAR1 gene between cells electroporated with plasmids encoding for a pool of 13 gRNAs against the IFNAR1 CpG island plus the dCas9:K+dCas9:D3A+dCas9:D3L ATRs (18 days post treatment) and untreated cells. The relative expression level of the IFNAR1 gene was normalised to the expression of DNMT1, and represented as fold change relative to the untreated cells (calibrator) (n=1). FIG. 20F. Top: Schematic of the Vascular Endothelial Growth Factor A (VEGFA) gene. The green box highlights a CpG island (the number of CpG residues are indicated) at the level of the gene promoter/enhancer region. Bottom: Histogram showing the fold change in the expression level of the VEGFA gene between cells electroporated with plasmids encoding for a pool of 3 gRNAs against the VEGFA CpG island plus the dCas9:K+dCas9:D3A+dCas9:D3L ATRs (14 days post treatment) and untreated cells. The relative expression level of the VEGFA gene was normalised to the expression of DNMT1, and represented as fold change relative to the untreated cells (calibrator) (n=1).

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F., and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J., and Kahn, A. (1996) DNA Isolation and Sequencing: Essential Techniques, John Wiley & Sons; Polak, J. M., and McGee, J. O'D. (1990) In Situ Hybridization: Principles and Practice, Oxford University Press; Gait, M. J. (1984) Oligonucleotide Synthesis: A PracticalApproach, IRL Press; and Lilley, D. M., and Dahlberg, J. E. (1992) Methods in Enzymology: DNA Structures Part A: Synthesis and Physical Analysis of DNA, Academic Press. Each of these general texts is herein incorporated by reference.

In one aspect, the present invention provides a product comprising two or more artificial transcription repressors (ATRs), or polynucleotides encoding therefor, selected from groups (a), (b) or (c):
(a) an ATR comprising a DNA-binding domain operably linked to a KRAB domain or homologue thereof;
(b) an ATR comprising a DNA-binding domain operably linked to a DNMT3A, DNMT3B or DNMT1 domain or homologue thereof; and
(c) an ATR comprising a DNA-binding domain operably linked to a DNMT3L domain or homologue thereof
   wherein at least two of the ATRs are selected from different groups (a), (b) or (c).

The product of the present invention may, for example, be a composition (e.g. a pharmaceutical composition) comprising two or more ATRs, or polynucleotides encoding therefor, selected from groups (a), (b) or (c): (a) an ATR comprising a DNA-binding domain operably linked to a KRAB domain or homologue thereof; (b) an ATR comprising a DNA-binding domain operably linked to a DNMT3A, DNMT3B or DNMT1 domain or homologue thereof; and (c) an ATR comprising a DNA-binding domain operably linked to a DNMT3L domain or homologue thereof, in admixture, wherein at least two of the ATRs are selected from different groups (a), (b) or (c). Alternatively, the product may, for example, be a kit comprising a preparation of two or more ATRs, or polynucleotides encoding therefor, selected from groups (a), (b) or (c): (a) an ATR comprising a DNA-binding domain operably linked to a KRAB domain or homologue thereof; (b) an ATR comprising a DNA-binding domain operably linked to a DNMT3A, DNMT3B or DNMT1 domain or homologue thereof; and (c) an ATR comprising a DNA-binding domain operably linked to a DNMT3L domain or homologue thereof, wherein at least two of the ATRs are selected from different groups (a), (b) or (c), and, optionally, instructions for the simultaneous, sequential or separate administration of the preparations to a subject in need thereof.

Artificial transcription repressors (ATRs) are agents that act to reduce the transcription of a target gene. ATRs may be chimeric proteins that are comprised of a DNA-binding domain operably linked to an effector domain (e.g. a KRAB domain, a DNMT3A, DNMT3B or DNMT1 domain or a DNMT3L domain, or homologues thereof). The DNA-binding domain enables binding of the ATR to a specific nucleic acid sequence, and may be engineered to bind to a nucleic acid sequence of choice. The effector domain may harbour a catalytic activity which enables repression of transcription of the target gene. Alternatively, or additionally, the effector domain may recruit additional agents within the cell to the target gene, which results in the repression of transcription of the target gene.

By “operably linked”, it is to be understood that the individual components are linked together in a manner which enables them to carry out their function (e.g. binding to DNA, catalysing a reaction or recruiting additional agents from within a cell) substantially unhindered. For example, a DNA-binding domain may be conjugated to an effector domain, for example to form a fusion protein.

Methods for conjugating polypeptides are known in the art, for example through the provision of a linker amino acid sequence connecting the polypeptides. Alternative methods of conjugating polypeptides known in the art include chemical and light-induced conjugation methods (e.g. using chemical cross-linking agents). Preferably, the DNA-binding domain and effector domain (e.g. KRAB domain, DNMT3A, DNMT3B or DNMT1 domain or DNMT3L domain, or homologue thereof) of the ATR form a fusion protein.

### Effector Domains

The term “effector domain”, is to be understood as referring to the part of the ATR which provides for the silencing effect on a target gene, for example by catalysing a reaction on the DNA or chromatin (e.g. methylation of DNA), or by recruiting an additional agent from within a cell, resulting in the repression of the transcription of a gene.

“Domain” is to be understood in this context as referring to a part of the ATR that harbours a certain function. The domain may be an individual domain (e.g. a catalytic domain) isolated from a natural protein or it may be an entire, full-length natural protein. Put another way, either the full-length protein or a functional fragment thereof can be used as an effector domain. Therefore, for example, “KRAB domain” refers to the part of the ATR that comprises an amino acid sequence with the function of a KRAB domain.

Chromatin remodelling enzymes that are known to be involved in the permanent epigenetic silencing of endogenous retroviruses (ERVs; Feschotte, C. et al. (2012) Nat. Rev. Genet. 13: 283-96; Leung, D. C. et al. (2012) Trends Biochem. Sci. 37: 127-33) may provide suitable effector domains for exploitation in the present invention.

The family of the Kruppel-associated box containing zinc finger proteins (KRAB-ZFP; Huntley, S. et al. (2006) Genome Res. 16: 669-77) plays an important role in the silencing of endogenous retroviruses. These transcription factors bind to specific ERV sequences through their ZFP DNA binding domain, while they recruit the KRAB Associated Protein 1 (KAP1) with their conserved KRAB domain. KAP1 in turn binds a large number of effectors that promote the local formation of repressive chromatin (lyengar, S. et al. (2011) J. Biol. Chem. 286: 26267-76).

In the early embryonic development, KAP1 is known to recruit SET domain bifurcated 1 (SETDB1), a histone methyltransferase that deposits histone H3 lysine-9 di- and tri-methylation (H3K9me2 and H3K9me3, respectively), two histone marks associated with transcriptional repression. Concurrently, KAP1 binds to Heterochromatin Protein 1 alpha (HP1α), which reads H3K9me2 and H3K9me3 and stabilises the KAP1-containing complex. KAP1 can also interact with other well known epigenetic silencers, such as the lysine-specific histone demethylase 1 (LSD1) that inhibits transcription by removing histone H3 lysine-4 methylation, and the nucleosome remodelling and deacetylase complex (NURD), which removes acetyl groups from histones. Finally, the KAP1-containing complex contributes to the recruitment of the de novo DNA methyltransferase 3A (DNMT3A), which methylates cytosines at CpG sites (Jones, P. A. (2012) Nat. Rev. Genet. 13: 484-92). Together, these data suggest a model in which, in the pre-implantation embryo, the KAP1-complex ensures ERV silencing through the concerted action of histone modifying enzymes and DNA methylation. Then, after implantation, the DNA methylation previously targeted by KRAB-ZFPs to the ERVs becomes stable (Reik, W. (2007) Nature 447: 425-32), being inherited throughout mitosis and somatic cell differentiation without the need of the continuous expression of ERVs-specific KRAB-ZFPs. Contrary to embryonic stem cells, the KAP1-complex is not able to efficiently induce DNA methylation in somatic cells, being only able to deposit H3K9 methylation. However, this histone mark is not maintained without being continuously deposited at the targeted site by the KRAB-ZFPs (Hathaway, N. A. et al. (2012) Cell 149: 1447-60).

Therefore, in view of an epigenetic therapy approach based on the transient expression of ATRs in somatic cells, the KRAB-ZFPs/KAP1 machinery is expected not to be functional if employed alone. On the other hand, we consider a preferable strategy to co-deliver two distinct ATRs: one based on, for example, the KRAB domain, the initiator of the epigenetic cascade occurring at ERVs in embryonic stem cells, and the other based on, for example, DNMT3A, the final lock of this process. This approach may allow recapitulating on a pre-selected target gene those repressive chromatin states established at ERVs in the pre-implantation embryo and then permanently inherited throughout mammalian development and adult life.

An ATR of the present invention may, for example, comprise a KRAB domain. Various KRAB domains are known in the family of KRAB-ZFP proteins. For example, an ATR of the present invention may comprise the KRAB domain of human zinc finger protein 10 (ZNF10; Szulc, J. et al. (2006) Nat. Methods 3: 109-16):

(SEQ ID NO: 1)
ALSPQHSAVTQGSIIKNKEGMDAKSLTAWSRTLVTFKDVFVDFTREEWKL
LDTAQQIVYRNVMLENYKNLVSLGYQLTKPDVILRLEKGEEPWLVEREIH
QETHPDSETAFEIKSSV

Further examples of suitable KRAB domains for use in the present invention include:

(the KRAB domain of the ZNF350 protein;
SEQ ID NO: 2)
ITLEDVAVDFTWEEWQLLGAAQKDLYRDVMLENYSNLVAVGYQASKPDAL
FKLEQGEQLWTIEDGIHSGACS
(the KRAB domain of the ZNF197 protein;
SEQ ID NO: 3)
VMFEEVSVCFTSEEWACLGPIQRALYWDVMLENYGNVTSLEWETMTENEE
VTSKPSSSQRADSHKGTSKRLQG
(the KRAB domain of the RBAK protein;
SEQ ID NO: 4)
VSFKDVAVDFTQEEWQQLDPDEKITYRDVMLENYSHLVSVGYDTTKPNVI
IKLEQGEEPWIMGGEFPCQHSP
(the KRAB domain of the ZKSCAN1 protein;
SEQ ID NO: 5)
VKIEDMAVSLILEEWGCQNLARRNLSRDNRQENYGSAFPQGGENRNENEE
STSKAETSEDSASRGETTGRSQKE
(the KRAB domain of the KRBOX4 protein;
SEQ ID NO: 6)
LTFKDVFVDFTLEEWQQLDSAQKNLYRDVMLENYSHLVSVGYLVAKPDVI
FRLGPGEESWMADGGTPVRTCA
(the KRAB domain of the ZNF274 protein;
SEQ ID NO: 7)
VTFEDVTLGFTPEEWGLLDLKQKSLYREVMLENYRNLVSVEHQLSKPDVV
SQLEEAEDFWPVERGIPQDTIP

An ATR of the present invention may, for example, comprise a domain of human DNA methyltransferase 3A (DNMT3A; Law, J. A. et al. (2010) Nat. Rev. Genet. 11: 204-20), preferably the catalytic domain. For example, an ATR of the present invention may comprise the sequence:

(SEQ ID NO: 8)
TYGLLRRREDWPSRLQMFFANNHDQEFDPPKVYPPVPAEKRKPIRVLSLF
DGIATGLLVLKDLGIQVDRYIASEVCEDSITVGMVRHQGKIMYVGDVRSV
TQKHIQEWGPFDLVIGGSPCNDLSIVNPARKGLYEGTGRLFFEFYRLLHD
ARPKEGDDRPFFWLFENVVAMGVSDKRDISRFLESNPVMIDAKEVSAAHR
ARYFWGNLPGMNRPLASTVNDKLELQECLEHGRIAKFSKVRTITTRSNSI
KQGKDQHFPVFMNEKEDILWCTEMERVFGFPVHYTDVSNMSRLARQRLLG
RSWSVPVIRHLFAPLKEYFACV

DNA methyltransferases 3B and 1 (DNMT3B and DNMT1), similarly to DNMT3A, are also responsible for the deposition and maintenance of DNA methylation, and may also be used in an ATR of the present invention. For example, an ATR of the present invention may comprise any of the sequences:

(the catalytic domain of human DNMT3B;
SEQ ID NO: 9)
CHGVLRRRKDWNVRLQAFFTSDTGLEYEAPKLYPAIPAARRRPIRVLSLF
DGIATGYLVLKELGIKVGKYVASEVCEESIAVGTVKHEGNIKYVNDVRNI
TKKNIEEWGPFDLVIGGSPCNDLSNVNPARKGLYEGTGRLFFEFYHLLNY
SRPKEGDDRPFFWMFENVVAMKVGDKRDISRFLECNPVMIDAIKVSAAHR
ARYFWGNLPGMNRPVIASKNDKLELQDCLEYNRIAKLKKVQTITTKSNSI
KQGKNQLFPVVMNGKEDVLWCTELERIFGFPVHYTDVSNMGRGARQKLLG
RSWSVPVIRHLFAPLKDYFACE
(DNMT3B: SEQ ID NO: 36)
MVAELISEEDLEFMKGDTRHLNGEEDAGGREDSILVNGACSDQSSDSPPI
LEAIRTPEIRGRRSSSRLSKREVSSLLSYTQDLTGDGDGEDGDGSDTPVM
PKLFRETRTRSESPAVRTRNNNSVSSRERHRPSPRSTRGRQGRNHVDESP
VEFPATRSLRRRATASAGTPWPSPPSSYLTIDLTDDTEDTHGTPQSSSTP
YARLAQDSQQGGMESPQVEADSGDGDSSEYQDGKEFGIGDLVWGKIKGFS
WWPAMVVSWKATSKRQAMSGMRWVQWFGDGKFSEVSADKLVALGLFSQHF
NLATFNKLVSYRKAMYHALEKARVRAGKTFPSSPGDSLEDQLKPMLEWAH
GGFKPTGIEGLKPNNTQPENKTRRRTADDSATSDYCPAPKRLKTNCYNNG
KDRGDEDQSREQMASDVANNKSSLEDGCLSCGRKNPVSFHPLFEGGLCQT
CRDRFLELFYMYDDDGYQSYCTVCCEGRELLLCSNTSCCRCFCVECLEVL
VGTGTAAEAKLQEPWSCYMCLPQRCHGVLRRRKDWNVRLQAFFTSDTGLE
YEAPKLYPAIPAARRRPIRVLSLFDGIATGYLVLKELGIKVGKYVASEVC
EESIAVGTVKHEGNIKYVNDVRNITKKNIEEWGPFDLVIGGSPCNDLSNV
NPARKGLYEGTGRLFFEFYHLLNYSRPKEGDDRPFFWMFENVVAMKVGDK
RDISRFLECNPVMIDAIKVSAAHRARYFWGNLPGMNRPVIASKNDKLELQ
DCLEYNRIAKLKKVQTITTKSNSIKQGKNQLFPVVMNGKEDVLWCTELER
IFGFPVHYTDVSNMGRGARQKLLGRSWSVPVIRHLFAPLKDYFACE
(the catalytic domain of human DNMT1;
SEQ ID NO: 10)
LRTLDVFSGCGGLSEGFHQAGISDTLWAIEMWDPAAQAFRLNNPGSTVFT
EDCNILLKLVMAGETTNSRGQRLPQKGDVEMLCGGPPCQGFSGMNRFNSR
TYSKFKNSLVVSFLSYCDYYRPRFFLLENVRNFVSFKRSMVLKLTLRCLV
RMGYQCTFGVLQAGQYGVAQTRRRAIILAAAPGEKLPLFPEPLHVFAPRA
CQLSVVVDDKKFVSNITRLSSGPFRTITVRDTMSDLPEVRNGASALEISY
NGEPQSWFQRQLRGAQYQPILRDHICKDMSALVAARMRHIPLAPGSDWRD
LPNIEVRLSDGTMARKLRYTHHDRKNGRSSSGALRGVCSCVEAGKACDPA
ARQFNTLIPWCLPHTGNRHNHWAGLYGRLEWDGFFSTTVTNPEPMGKQGR
VLHPEQHRVVSVRECARSQGFPDTYRLFGNILDKHRQVGNAVPPPLAKAI
GLEIKLCMLAKARESASAKIKEEEAAKD

An ATR of the present invention may, for example, comprise DNA (cytosine-5)-methyltransferase 3-like (DNMT3L), a catalytically inactive DNA methyltransferase that activates DNMT3A by binding to its catalytic domain. For example, an ATR of the present invention may comprise the sequence:

(SEQ ID NO: 11)
MAAIPALDPEAEPSMDVILVGSSELSSSVSPGTGRDLIAYEVKANQRNIE
DICICCGSLQVHTQHPLFEGGICAPCKDKFLDALFLYDDDGYQSYCSICC
SGETLLICGNPDCTRCYCFECVDSLVGPGTSGKVHAMSNWVCYLCLPSSR
SGLLQRRRKWRSQLKAFYDRESENPLEMFETVPVWRRQPVRVLSLFEDIK
KELTSLGFLESGSDPGQLKHVVDVTDTVRKDVEEWGPFDLVYGATPPLGH
TCDRPPSWYLFQFHRLLQYARPKPGSPRPFFWMFVDNLVLNKEDLDVASR
FLEMEPVTIPDVHGGSLQNAVRVWSNIPAIRSRHWALVSEEELSLLAQNK
QSSKLAAKWPTKLVKNCFLPLREYFKYFSTELTSSL

An ATR of the present invention may, for example, comprise a SETDB1 domain. For example, an ATR of the present invention may comprise any of the sequences:

(SEQ ID NO: 12)
MSSLPGCIGLDAATATVESEEIAELQQAVVEELGISMEELRHFIDEELEK
MDCVQQRKKQLAELETWVIQKESEVAHVDQLFDDASRAVTNCESLVKDFY
SKLGLQYRDSSSEDESSRPTEIIEIPDEDDDVLSIDSGDAGSRTPKDQKL
REAMAALRKSAQDVQKFMDAVNKKSSSQDLHKGTLSQMSGELSKDGDLIV
SMRILGKKRTKTWHKGTLIAIQTVGPGKKYKVKFDNKGKSLLSGNHIAYD
YHPPADKLYVGSRVVAKYKDGNQVWLYAGIVAETPNVKNKLRFLIFFDDG
YASYVTQSELYPICRPLKKTWEDIEDISCRDFIEEYVTAYPNRPMVLLKS
GQLIKTEWEGTWVVKSRVEEVDGSLVRILFLDDKRCEWIYRGSTRLEPMF
SMKTSSASALEKKQGQLRTRPNMGAVRSKGPVVQYTQDLTGTGTQFKPVE
PPQPTAPPAPPFPPAPPLSPQAGDSDLESQLAQSRKQVAKKSTSFRPGSV
GSGHSSPTSPALSENVSGGKPGINQTYRSPLGSTASAPAPSALPAPPAPP
VFHGMLERAPAEPSYRAPMEKLFYLPHVCSYTCLSRVRPMRNEQYRGKNP
LLVPLLYDFRRMTARRRVNRKMGFHVIYKTPCGLCLRTMQEIERYLFETG
CDFLFLEMFCLDPYVLVDRKFQPYKPFYYILDITYGKEDVPLSCVNEIDT
TPPPQVAYSKERIPGKGVFINTGPEFLVGCDCKDGCRDKSKCACHQLTIQ
ATACTPGGQINPNSGYQYKRLEECLPTGVYECNKRCKCDPNMCTNRLVQH
GLQVRLQLFKTQNKGWGIRCLDDIAKGSFVCIYAGKILTDDFADKEGLEM
GDEYFANLDHIESVENFKEGYESDAPCSSDSSGVDLKDQEDGNSGTEDPE
ESNDDSSDDNFCKDEDFSTSSVWRSYATRRQTRGQKENGLSETTSKDSHP
PDLGPPHIPVPPSIPVGGCNPPSSEETPKNKVASWLSCNSVSEGGFADSD
SHSSFKTNEGGEGRAGGSRMEAEKASTSGLGIKDEGDIKQAKKEDTDDRN
KMSVVTESSRNYGYNPSPVKPEGLRRPPSKTSMHQSRRLMASAQSNPDDV
LTLSSSTESEGESGTSRKPTAGQTSATAVDSDDIQTISSGSEGDDFEDKK
NMTGPMKRQVAVKSTRGFALKSTHGIAIKSTNMASVDKGESAPVRKNTRQ
FYDGEESCYIIDAKLEGNLGRYLNHSCSPNLFVQNVFVDTHDLRFPWVAF
FASKRIRAGTELTVVDYNYEVGSVEGKELLCCCGAIECRGRLL
(the catalytic domain of human SETDB1;
SEQ ID NO: 13)
VGCDCKDGCRDKSKCACHQLTIQATACTPGGQINPNSGYQYKRLEECLPT
GVYECNKRCKCDPNMCTNRLVQHGLQVRLQLFKTQNKGWGIRCLDDIAKG
SFVCIYAGKILTDDFADKEGLEMGDEYFANLDHIESVENFKEGYESDAPC
SSDSSGVDLKDQEDGNSGTEDPEESNDDSSDDNFCKDEDFSTSSVWRSYA
TRRQTRGQKENGLSETTSKDSHPPDLGPPHIPVPPSIPVGGCNPPSSEET
PKNKVASWLSCNSVSEGGFADSDSHSSFKTNEGGEGRAGGSRMEAEKAST
SGLGIKDEGDIKQAKKEDTDDRNKMSVVTESSRNYGYNPSPVKPEGLRRP
PSKTSMHQSRRLMASAQSNPDDVLTLSSSTESEGESGTSRKPTAGQTSAT
AVDSDDIQTISSGSEGDDFEDKKNMTGPMKRQVAVKSTRGFALKSTHGIA
IKSTNMASVDKGESAPVRKNTRQFYDGEESCYIIDAKLEGNLGRYLNHSC
SPNLFVQNVFVDTHDLRFPWVAFFASKRIRAGTELTWDYNYEVGSVEGKE
LLCCCGAIECRGRLL

The ATR of the present invention may, for example, comprise an amino acid sequence that has 50%, 60%, 70%, 80%, 90%, 95%, 99% or 100% identity to SEQ ID NOs: 1, 2, 3, 4, 5, 6, 7, 8, 9, 10, 11, 12 or 13 wherein the amino acid sequence substantially retains the natural function of the protein represented by SEQ ID NOs: 1, 2, 3, 4, 5, 6, 7, 8, 9, 10, 11, 12 or 13.

The ATR of the present invention may, for example, be encoded by a polynucleotide comprising a nucleic acid sequence which encodes the protein of SEQ ID NOs: 1, 2, 3, 4, 5, 6, 7, 8, 9, 10, 11, 12 or 13, or a protein that has 50%, 60%, 70%, 80%, 90%, 95%, 99% or 100% amino acid identity to SEQ ID NOs: 1, 2, 3, 4, 5, 6, 7, 8, 9, 10, 11, 12 or 13 wherein the amino acid sequence substantially retains the natural function of the protein represented by SEQ ID NOs: 1, 2, 3, 4, 5, 6, 7, 8, 9, 10, 11, 12 or13.

The ATR of the present invention may, for example, comprise an amino acid sequence that has at least 50%, 60%, 70%, 80%, 90%, 95%, 99% or 100% identity to SEQ ID NOs: 1, 2, 3, 4, 5, 6, 7, 8, 9, 10, 11, 12 or 13 wherein the amino acid sequence substantially retains the natural function of the protein represented by SEQ ID NOs: 1, 2, 3, 4, 5, 6, 7, 8, 9, 10, 11, 12 or 13.

The ATR of the present invention may, for example, be encoded by a polynucleotide comprising a nucleic acid sequence which encodes the protein of SEQ ID NOs: 1, 2, 3, 4, 5, 6, 7, 8, 9, 10, 11, 12 or 13, or a protein that has at least 50%, 60%, 70%, 80%, 90%, 95%, 99% or 100% amino acid identity to SEQ ID NOs: 1, 2, 3, 4, 5, 6, 7, 8, 9, 10, 11, 12 or 13 wherein the amino acid sequence substantially retains the natural function of the protein represented by SEQ ID NOs: 1, 2, 3, 4, 5, 6, 7, 8, 9, 10, 11, 12 or13.

### DNA-Binding Domains

The ATRs of the invention comprise a DNA-binding domain which binds a specific nucleic acid sequence and enables the ATR to be targeted to specific site in a polynucleotide, for example the genome of a cell. The DNA-binding domain may, for example, be protein-, DNA-, RNA- or chemical-based.

A number of suitable DNA-binding domains are known in the art, for example transcription-activator like effector (TALE) domains and zinc finger proteins (ZFPs) (Gaj, T. et al. (2013) Trends Biotechnol. 31: 397-405).

The tetracycline-controlled repressor (tetR) DNA-binding domain, for example the E. coli tetR DNA-binding domain (Gossen, M. et al. (1992) Proc. Natl. Acad. Sci. USA 89: 5547-51), may also be employed as a suitable DNA-binding domain in the ATRs of the present invention. The tetR system is particularly advantageous for use in model systems, because it allows temporal control of binding of tetR to its target nucleotide sequence, the tetracycline operon (TetO), by doxycycline (doxy) administration. This allows investigation of whether the chromatin states induced by the ATRs can be maintained after the release of the ATRs from their target locus.

In addition, methods for the engineering of DNA-binding domains to bind to desired nucleic acid sequences are known in the art.

Example sequences of suitable TALE domains include:

(SEQ ID NO: 14)
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPEQVVAIASNIGGKQALETVQRLLPVLCQAHGL
TPEQVVAIASHDGGKQALETVQRLLPVLCQAHGLTPEQVVAIASHDGGKQ
ALETVQRLLPVLCQAHGLTPEQVVAIASHDGGKQALETVQRLLPVLCQAH
GLTPEQVVAIASNIGGKQALETVQRLLPVLCQAHGLTPEQVVAIASNNGG
KQALETVQRLLPVLCQAHGLTPEQVVAIASNIGGKQALETVQRLLPVLCQ
AHGLTPEQVVAIASNGGGKQALETVQRLLPVLCQAHGLTPEQVVAIASNG
GGKQALETVQRLLPVLCQAHGLTPEQVVAIASNNGGKQALETVQRLLPVL
CQAHGLTPEQVVAIASNNGGKQALETVQRLLPVLCQAHGLTPEQVVAIAS
HDGGKQALETVQRLLPVLCQAHGLTPEQVVAIASHDGGKQALETVQRLLP
VLCQAHGLTPEQVVAIASHDGGKQALETVQRLLPVLCQAHGLTPEQVVAI
ASHDGGKQALETVQRLLPVLCQAHGLTPEQVVAIASNIGGKQALETVQRL
LPVLCQAHGLTPEQVVAIASHDGGKQALETVQRLLPVLCQAHGLTPQQWA
IASNGGGRPALESIVAQLSRPDPALAALTNDHLVALACLGGRPALDAVKK
GLPHAPALIKRTNRRIPERTSHRVA,
which targets the binding site:
(SEQ ID NO: 34)
5′-TACCCAGATTGGCCCCACT-3′
and:
(SEQ ID NO: 15)
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPEQVVAIASNIGGKQALETVQRLLPVLCQAHGL
TPEQVVAIASHDGGKQALETVQRLLPVLCQAHGLTPEQVVAIASHDGGKQ
ALETVQRLLPVLCQAHGLTPEQVVAIASNGGGKQALETVQRLLPVLCQAH
GLTPEQVVAIASNIGGKQALETVQRLLPVLCQAHGLTPEQVVAIASNNGG
KQALETVQRLLPVLCQAHGLTPEQVVAIASNIGGKQALETVQRLLPVLCQ
AHGLTPEQVVAIASNNGGKQALETVQRLLPVLCQAHGLTPEQVVAIASNN
GGKQALETVQRLLPVLCQAHGLTPEQVVAIASNIGGKQALETVQRLLPVL
CQAHGLTPEQVVAIASNNGGKQALETVQRLLPVLCQAHGLTPEQVVAIAS
NIGGKQALETVQRLLPVLCQAHGLTPEQVVAIASNIGGKQALETVQRLLP
VLCQAHGLTPEQVVAIASNIGGKQALETVQRLLPVLCQAHGLTPEQVVAI
ASNNGGKQALETVQRLLPVLCQAHGLTPEQVVAIASNNGGKQALETVQRL
LPVLCQAHGLTPEQVVAIASNGGGKQALETVQRLLPVLCQAHGLTPQQWA
IASNGGGRPALESIVAQLSRPDPALAALTNDHLVALACLGGRPALDAVKK
GLPHAPALIKRTNRRIPERTSHRVA,
which targets the binding site:
(SEQ ID NO: 35)
5′-TACCTAGAGGAGAAAGGTT-3′

Example sequences of TALE domains that have been designed to target the promoter region of the β2-microglobulin gene include:

(SEQ ID NO: 16)
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASHDGGKQALETVQRLLPVLCQDHGL
TPDQVVAIASNGGGKQALETVQRLLPVLCQDHGLTPDQVVAIASHDGGKQ
ALETVQRLLPVLCQDHGLTPDQVVAIASNGGGKQALETVQRLLPVLCQDH
GLTPDQVVAIASHDGGKQALETVQRLLPVLCQDHGLTPDQVVAIASHDGG
KQALETVQRLLPVLCQDHGLTPDQVVAIASNGGGKQALETVQRLLPVLCQ
DHGLTPDQVVAIASNIGGKQALETVQRLLPVLCQDHGLTPDQVVAIASHD
GGKQALETVQRLLPVLCQDHGLTPDQVVAIASHDGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASNHGGKQALETVQRL
LPVLCQDHGLTPDQVVAIASHDGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG,
which targets the binding site:
(SEQ ID NO: 17)
5′-TCTCTCCTACCCTCCCGCT-3′
(SEQ ID NO: 18)
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASNNGGKQALETVQRLLPVLCQDHGL
TPDQVVAIASNNGGKQALETVQRLLPVLCQDHGLTPDQVVAIASNGGGKQ
ALETVQRLLPVLCQDHGLTPDQVVAIASHDGGKQALETVQRLLPVLCQDH
GLTPDQVVAIASHDGGKQALETVQRLLPVLCQDHGLTPDQVVAIASNGGG
KQALETVQRLLPVLCQDHGLTPDQVVAIASNGGGKQALETVQRLLPVLCQ
DHGLTPDQVVAIASHDGGKQALETVQRLLPVLCQDHGLTPDQVVAIASHD
GGKQALETVQRLLPVLCQDHGLTPDQVVAIASNGGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG,
which targets the binding site:
(SEQ ID NO: 19)
5′-TGGTCCTTCCTCTCCCGCT-3′
(SEQ ID NO: 20)
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASHDGGKQALETVQRLLPVLCQDHGL
TPDQVVAIASNNGGKQALETVQRLLPVLCQDHGLTPDQVVAIASHDGGKQ
ALETVQRLLPVLCQDHGLTPDQVVAIASNGGGKQALETVQRLLPVLCQDH
GLTPDQVVAIASHDGGKQALETVQRLLPVLCQDHGLTPDQVVAIASHDGG
KQALETVQRLLPVLCQDHGLTPDQVVAIASNNGGKQALETVQRLLPVLCQ
DHGLTPDQVVAIASNGGGKQALETVQRLLPVLCQDHGLTPDQVVAIASNN
GGKQALETVQRLLPVLCQDHGLTPDQVVAIASNIGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASNGGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASHDGGKQALETVQRL
LPVLCQDHGLTPDQVVAIASNGGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG,
which targets the binding site:
(SEQ ID NO: 21)
5′-TCGCTCCGTGACTTCCCTT-3′

Example sequences of TALE domains that have been designed to target the BCL11A gene include:

TALE BCL11A #1
(SEQ ID NO: 37)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASNGGGKQALETVQRLLPVLCQDHGLTPDQVVAIASNN
GGKQALETVQRLLPVLCQDHGLTPDQVVAIASNNGGKQALETVQRLLPVLCQDHGL
TPDQVVAIASNGGGKQALETVQRLLPVLCQDHGLTPDQVVAIASHDGGKQALETVQ
RLLPVLCQDHGLTPDQVVAIASHDGGKQALETVQRLLPVLCQDHGLTPDQVVAIAS
NNGGKQALETVQRLLPVLCQDHGLTPDQVVAIASHDGGKQALETVQRLLPVLCQD
HGLTPDQVVAIASHDGGKQALETVQRLLPVLCQDHGLTPDQVVAIASHDGGKQALE
TVQRLLPVLCQDHGLTPDQVVAIASHDGGKQALETVQRLLPVLCQDHGLTPDQVVA
IASHDGGKQALETVQRLLPVLCQDHGLTPDQVVAIASNIGGKQALETVQRLLPVLCQ
DHGLTPDQVVAIASNNGGKQALETVQRLLPVLCQDHGLTPDQVVAIASHDGGKQAL
ETVQRLLPVLCQDHGLTPDQVVAIASNIGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 38)
5′-TCCAAAAGCCAGTCTCACC-3′
TALE BCL11A #2
(SEQ ID NO: 39)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASNNGGKQALETVQRLLPVLCQDHGLTPDQVVAIASHDGGKQALETVQRL
LPVLCQDHGLTPDQVVAIASHDGGKQALETVQRLLPVLCQDHGLTPDQVVAIASNN
GGKQALETVQRLLPVLCQDHGLTPDQVVAIASNIGGKQALETVQRLLPVLCQDHGL
TPDQVVAIASNNGGKQALETVQRLLPVLCQDHGLTPDQVVAIASNIGGKQALETVQ
RLLPVLCQDHGLTPDQVVAIASHDGGKQALETVQRLLPVLCQDHGLTPDQVVAIAS
HDGGKQALETVQRLLPVLCQDHGLTPDQVVAIASNGGGKQALETVQRLLPVLCQD
HGLTPDQVVAIASHDGGKQALETVQRLLPVLCQDHGLTPDQVVAIASNGGGKQALE
TVQRLLPVLCQDHGLTPDQVVAIASNGGGKQALETVQRLLPVLCQDHGLTPDQVVA
IASHDGGKQALETVQRLLPVLCQDHGLTPDQVVAIASNGGGKQALETVQRLLPVLC
QDHGLTPDQVVAIASHDGGKQALETVQRLLPVLCQDHGLTPDQVVAIASNNGGKQ
ALETVQRLLPVLCQDHGLTPDQVVAIASNIGGKQALESIVAQLSRPDPALAALTNDHL
VALACLGGRPALDAVKKGLPHAPALIKRTNRRIPERTSHRVAGSGGG,
which targets the binding site:
(SEQ ID NO: 40)
5′-TCTCCCCGGGAATCGTTTT-3′
TALE BCL11A #3
(SEQ ID NO: 41)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASNNGGKQALETVQRL
LPVLCQDHGLTPDQVVAIASNNGGKQALETVQRLLPVLCQDHGLTPDQVVAIASHD
GGKQALETVQRLLPVLCQDHGLTPDQVVAIASNGGGKQALETVQRLLPVLCQDHG
LTPDQVVAIASNGGGKQALETVQRLLPVLCQDHGLTPDQVVAIASNGGGKQALETV
QRLLPVLCQDHGLTPDQVVAIASNNGGKQALETVQRLLPVLCQDHGLTPDQVVAIA
SHDGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALET
VQRLLPVLCQDHGLTPDQVVAIASNHGGKQALETVQRLLPVLCQDHGLTPDQVVAI
ASHDGGKQALETVQRLLPVLCQDHGLTPDQVVAIASNIGGKQALETVQRLLPVLCQ
DHGLTPDQVVAIASNGGGKQALETVQRLLPVLCQDHGLTPDQVVAIASNGGGKQAL
ETVQRLLPVLCQDHGLTPDQVVAIASNGGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 42)
5′-TCCTCCCGCTGCACACTTG-3′
TALE BCL11A #4
(SEQ ID NO: 43)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASNNGGKQALETVQRLLPVLCQDHGLTPDQVVAIASHDGGKQALETVQRL
LPVLCQDHGLTPDQVVAIASNIGGKQALETVQRLLPVLCQDHGLTPDQVVAIASNIG
GKQALETVQRLLPVLCQDHGLTPDQVVAIASNIGGKQALETVQRLLPVLCQDHGLT
PDQVVAIASNNGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASN
NGGKQALETVQRLLPVLCQDHGLTPDQVVAIASNIGGKQALETVQRLLPVLCQDHG
LTPDQVVAIASNNGGKQALETVQRLLPVLCQDHGLTPDQVVAIASNGGGKQALETV
QRLLPVLCQDHGLTPDQVVAIASNGGGKQALETVQRLLPVLCQDHGLTPDQVVAIA
SNGGGKQALETVQRLLPVLCQDHGLTPDQVVAIASHDGGKQALETVQRLLPVLCQ
DHGLTPDQVVAIASNIGGKQALETVQRLLPVLCQDHGLTPDQVVAIASHDGGKQAL
ETVQRLLPVLCQDHGLTPDQVVAIASHDGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 44)
5′-TAGTCATCCCCACAATAGT-3′
TALE BCL11A #5
(SEQ ID NO: 45)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASNIGGKQALETVQRLLPVLCQDHGLTPDQVVAIASHDGGKQALETVQRLL
PVLCQDHGLTPDQVVAIASNIGGKQALETVQRLLPVLCQDHGLTPDQVVAIASNNG
GKQALETVQRLLPVLCQDHGLTPDQVVAIASNGGGKQALETVQRLLPVLCQDHGLT
PDQVVAIASNGGGKQALETVQRLLPVLCQDHGLTPDQVVAIASNNGGKQALETVQR
LLPVLCQDHGLTPDQVVAIASHDGGKQALETVQRLLPVLCQDHGLTPDQVVAIASH
DGGKQALETVQRLLPVLCQDHGLTPDQVVAIASHDGGKQALETVQRLLPVLCQDH
GLTPDQVVAIASNGGGKQALETVQRLLPVLCQDHGLTPDQVVAIASNNGGKQALET
VQRLLPVLCQDHGLTPDQVVAIASHDGGKQALETVQRLLPVLCQDHGLTPDQVVAI
ASNIGGKQALETVQRLLPVLCQDHGLTPDQVVAIASNIGGKQALETVQRLLPVLCQD
HGLTPDQVVAIASNIGGKQALETVQRLLPVLCQDHGLTPDQVVAIASNIGGKQALET
VQRLLPVLCQDHGLTPDQVVAIASNGGGKQALESIVAQLSRPDPALAALTNDHLVAL
ACLGGRPALDAVKKGLPHAPALIKRTNRRIPERTSHRVAGSGGG,
which targets the binding site:
(SEQ ID NO: 46)
5′-TCCCGCTGCCTTTTGTGCC-3′
TALE BCL11A #6
(SEQ ID NO: 47)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASNNGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNGGGKQALETVQ
RLLPVLCQDHGLTPDQVVAIASNNGGKQALETVQRLLPVLCQDHGLTPDQVVAIAS
NNGGKQALETVQRLLPVLCQDHGLTPDQVVAIASNNGGKQALETVQRLLPVLCQD
HGLTPDQVVAIASNGGGKQALETVQRLLPVLCQDHGLTPDQVVAIASNIGGKQALE
TVQRLLPVLCQDHGLTPDQVVAIASHDGGKQALETVQRLLPVLCQDHGLTPDQVVA
IASNGGGKQALETVQRLLPVLCQDHGLTPDQVVAIASNGGGKQALETVQRLLPVLC
QDHGLTPDQVVAIASNGGGKQALETVQRLLPVLCQDHGLTPDQVVAIASHDGGKQ
ALETVQRLLPVLCQDHGLTPDQVVAIASNGGGKQALESIVAQLSRPDPALAALTNDH
LVALACLGGRPALDAVKKGLPHAPALIKRTNRRIPERTSHRVAGSGGG,
which targets the binding site:
(SEQ ID NO: 48)
5′-TCCTCGCGCTTGCCCTCCC-3′
TALE BCL11A #7
(SEQ ID NO: 49)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVVAIASNG
GGKQALETVQRLLPVLCQDHGLTPDQVVAIASNGGGKQALETVQRLLPVLCQDHG
LTPDQVVAIASNNGGKQALETVQRLLPVLCQDHGLTPDQVVAIASNGGGKQALETV
QRLLPVLCQDHGLTPDQVVAIASHDGGKQALETVQRLLPVLCQDHGLTPDQVVAIA
SHDGGKQALETVQRLLPVLCQDHGLTPDQVVAIASNIGGKQALETVQRLLPVLCQD
HGLTPDQVVAIASHDGGKQALETVQRLLPVLCQDHGLTPDQVVAIASNIGGKQALET
VQRLLPVLCQDHGLTPDQVVAIASNHGGKQALETVQRLLPVLCQDHGLTPDQVVAI
ASHDGGKQALETVQRLLPVLCQDHGLTPDQVVAIASNGGGKQALETVQRLLPVLC
QDHGLTPDQVVAIASHDGGKQALETVQRLLPVLCQDHGLTPDQVVAIASNNGGKQ
ALETVQRLLPVLCQDHGLTPDQVVAIASNNGGKQALESIVAQLSRPDPALAALTNDH
LVALACLGGRPALDAVKKGLPHAPALIKRTNRRIPERTSHRVAGSGGG,
which targets the binding site:
(SEQ ID NO: 50)
5′-TCCCCCGGCCCTAGCTCCT-3′
TALE BCL11A #8
(SEQ ID NO: 51)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASNNGGKQALETVQRLLPVLCQDHGLTPDQVVAIASHDGGKQALETVQ
RLLPVLCQDHGLTPDQVVAIASNGGGKQALETVQRLLPVLCQDHGLTPDQVVAIAS
NNGGKQALETVQRLLPVLCQDHGLTPDQVVAIASNIGGKQALETVQRLLPVLCQDH
GLTPDQVVAIASHDGGKQALETVQRLLPVLCQDHGLTPDQVVAIASNGGGKQALET
VQRLLPVLCQDHGLTPDQVVAIASNHGGKQALETVQRLLPVLCQDHGLTPDQVVAI
ASHDGGKQALETVQRLLPVLCQDHGLTPDQVVAIASNHGGKQALETVQRLLPVLCQ
DHGLTPDQVVAIASHDGGKQALETVQRLLPVLCQDHGLTPDQVVAIASHDGGKQAL
ETVQRLLPVLCQDHGLTPDQVVAIASNGGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 52)
5′-TCCTGGTCCGCCCCCAGCA-3′
TALE BCL11A #9
(SEQ ID NO: 53)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASHDGGKQALETVQRLLPVLCQDHGLTPDQVVAIASNG
GGKQALETVQRLLPVLCQDHGLTPDQVVAIASNGGGKQALETVQRLLPVLCQDHG
LTPDQVVAIASNNGGKQALETVQRLLPVLCQDHGLTPDQVVAIASHDGGKQALETV
QRLLPVLCQDHGLTPDQVVAIASNGGGKQALETVQRLLPVLCQDHGLTPDQVVAIA
SNNGGKQALETVQRLLPVLCQDHGLTPDQVVAIASHDGGKQALETVQRLLPVLCQ
DHGLTPDQVVAIASHDGGKQALETVQRLLPVLCQDHGLTPDQVVAIASNIGGKQAL
ETVQRLLPVLCQDHGLTPDQVVAIASNIGGKQALETVQRLLPVLCQDHGLTPDQVV
AIASNIGGKQALETVQRLLPVLCQDHGLTPDQVVAIASHDGGKQALETVQRLLPVLC
QDHGLTPDQVVAIASNGGGKQALETVQRLLPVLCQDHGLTPDQVVAIASNGGGKQ
ALETVQRLLPVLCQDHGLTPDQVVAIASNGGGKQALESIVAQLSRPDPALAALTNDH
LVALACLGGRPALDAVKKGLPHAPALIKRTNRRIPERTSHRVAGSGGG,
which targets the binding site:
(SEQ ID NO: 54)
5′-TGCCGAGACCTCTTCTCGA-3′
TALE BCL11A #10
(SEQ ID NO: 55)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASNNGGKQALETVKRLLPVLCQDHGLTPDQVVAIASNNGGKQALETVQRL
LPVLCQDHGLTPDQVVAIASNNGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNGGGKQALETVQ
RLLPVLCQDHGLTPDQVVAIASHDGGKQALETVQRLLPVLCQDHGLTPDQVVAIAS
NIGGKQALETVQRLLPVLCQDHGLTPDQVVAIASHDGGKQALETVQRLLPVLCQDH
GLTPDQVVAIASNNGGKQALETVQRLLPVLCQDHGLTPDQVVAIASHDGGKQALET
VQRLLPVLCQDHGLTPDQVVAIASHDGGKQALETVQRLLPVLCQDHGLTPDQVVAI
ASNGGGKQALETVQRLLPVLCQDHGLTPDQVVAIASNGGGKQALETVQRLLPVLC
QDHGLTPDQVVAIASNGGGKQALETVQRLLPVLCQDHGLTPDQVVAIASHDGGKQ
ALETVQRLLPVLCQDHGLTPDQVVAIASNGGGKQALESIVAQLSRPDPALAALTNDH
LVALACLGGRPALDAVKKGLPHAPALIKRTNRRIPERTSHRVAGSGGG,
which targets the binding site:
(SEQ ID NO: 56)
5′-TCGGCTTTGCAAAGCATTT-3′
TALE BCL11A #11
(SEQ ID NO: 57)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNNGGKQALETVQ
RLLPVLCQDHGLTPDQVVAIASNNGGKQALETVQRLLPVLCQDHGLTPDQVVAIAS
NNGGKQALETVQRLLPVLCQDHGLTPDQVVAIASNIGGKQALETVQRLLPVLCQDH
GLTPDQVVAIASNIGGKQALETVQRLLPVLCQDHGLTPDQVVAIASNGGGKQALETV
QRLLPVLCQDHGLTPDQVVAIASHDGGKQALETVQRLLPVLCQDHGLTPDQVVAIA
SNNGGKQALETVQRLLPVLCQDHGLTPDQVVAIASNGGGKQALETVQRLLPVLCQ
DHGLTPDQVVAIASNGGGKQALETVQRLLPVLCQDHGLTPDQVVAIASNGGGKQAL
ETVQRLLPVLCQDHGLTPDQVVAIASNGGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 58)
5′-TGCAAAGCCGAGTTTCACC-3′
TALE BCL11A #12
(SEQ ID NO: 59)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASNIGGKQALETVQRLLPVLCQDHGLTPDQVVAIASNIG
GKQALETVQRLLPVLCQDHGLTPDQVVAIASNIGGKQALETVQRLLPVLCQDHGLT
PDQVVAIASNIGGKQALETVQRLLPVLCQDHGLTPDQVVAIASNHGGKQALETVQR
LLPVLCQDHGLTPDQVVAIASHDGGKQALETVQRLLPVLCQDHGLTPDQVVAIASH
DGGKQALETVQRLLPVLCQDHGLTPDQVVAIASNIGGKQALETVQRLLPVLCQDHG
LTPDQVVAIASNNGGKQALETVQRLLPVLCQDHGLTPDQVVAIASNGGGKQALETV
QRLLPVLCQDHGLTPDQVVAIASHDGGKQALETVQRLLPVLCQDHGLTPDQVVAIA
SNGGGKQALETVQRLLPVLCQDHGLTPDQVVAIASHDGGKQALETVQRLLPVLCQ
DHGLTPDQVVAIASNIGGKQALETVQRLLPVLCQDHGLTPDQVVAIASHDGGKQAL
ETVQRLLPVLCQDHGLTPDQVVAIASHDGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 60)
5′-TACAGTTGCCCTGCAAAAT-3′
TALE BCL11A #13
(SEQ ID NO: 61)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASNGGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASHDGGKQALETVQRLLPVLCQDHGLTPDQVVAIASNNGGKQALETVQ
RLLPVLCQDHGLTPDQVVAIASHDGGKQALETVQRLLPVLCQDHGLTPDQVVAIAS
NGGGKQALETVQRLLPVLCQDHGLTPDQVVAIASNNGGKQALETVQRLLPVLCQD
HGLTPDQVVAIASHDGGKQALETVQRLLPVLCQDHGLTPDQVVAIASNIGGKQALET
VQRLLPVLCQDHGLTPDQVVAIASHDGGKQALETVQRLLPVLCQDHGLTPDQVVAI
ASNIGGKQALETVQRLLPVLCQDHGLTPDQVVAIASHDGGKQALETVQRLLPVLCQ
DHGLTPDQVVAIASNGGGKQALETVQRLLPVLCQDHGLTPDQVVAIASNGGGKQAL
ETVQRLLPVLCQDHGLTPDQVVAIASNNGGKQALESIVAQLSRPDPALAALTNDHLV
ALACLGGRPALDAVKKGLPHAPALIKRTNRRIPERTSHRVAGSGGG,
which targets the binding site:
(SEQ ID NO: 62)
5′-TCCGCCCTGGGTACTTTCT-3′
TALE BCL11A #14
(SEQ ID NO: 63)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASNIGGKQALETVQRLLPVLCQDHGLTPDQVVAIASNNGGKQALETVQRLL
PVLCQDHGLTPDQVVAIASNGGGKQALETVQRLLPVLCQDHGLTPDQVVAIASHDG
GKQALETVQRLLPVLCQDHGLTPDQVVAIASNIGGKQALETVQRLLPVLCQDHGLT
PDQVVAIASNGGGKQALETVQRLLPVLCQDHGLTPDQVVAIASHDGGKQALETVQR
LLPVLCQDHGLTPDQVVAIASHDGGKQALETVQRLLPVLCQDHGLTPDQVVAIASH
DGGKQALETVQRLLPVLCQDHGLTPDQVVAIASHDGGKQALETVQRLLPVLCQDH
GLTPDQVVAIASNIGGKQALETVQRLLPVLCQDHGLTPDQVVAIASHDGGKQALETV
QRLLPVLCQDHGLTPDQVVAIASNIGGKQALETVQRLLPVLCQDHGLTPDQVVAIAS
NIGGKQALETVQRLLPVLCQDHGLTPDQVVAIASNGGGKQALETVQRLLPVLCQDH
GLTPDQVVAIASNIGGKQALETVQRLLPVLCQDHGLTPDQVVAIASNNGGKQALETV
QRLLPVLCQDHGLTPDQVVAIASNGGGKQALESIVAQLSRPDPALAALTNDHLVALA
CLGGRPALDAVKKGLPHAPALIKRTNRRIPERTSHRVAGSGGG,
which targets the binding site:
(SEQ ID NO: 64)
5′-TCTCTTGTCCACAGCTCGG-3′
TALE BCL11A #15
(SEQ ID NO: 65)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASHDGGKQALETVQRLLPVLCQDHGLTPDQVVAIASNN
GGKQALETVQRLLPVLCQDHGLTPDQVVAIASHDGGKQALETVQRLLPVLCQDHGL
TPDQVVAIASNGGGKQALETVQRLLPVLCQDHGLTPDQVVAIASNNGGKQALETVQ
RLLPVLCQDHGLTPDQVVAIASHDGGKQALETVQRLLPVLCQDHGLTPDQVVAIAS
HDGGKQALETVQRLLPVLCQDHGLTPDQVVAIASNGGGKQALETVQRLLPVLCQD
HGLTPDQVVAIASNGGGKQALETVQRLLPVLCQDHGLTPDQVVAIASNGGGKQALE
TVQRLLPVLCQDHGLTPDQVVAIASNGGGKQALETVQRLLPVLCQDHGLTPDQVVA
IASNNGGKQALETVQRLLPVLCQDHGLTPDQVVAIASNGGGKQALETVQRLLPVLC
QDHGLTPDQVVAIASNNGGKQALETVQRLLPVLCQDHGLTPDQVVAIASHDGGKQ
ALETVQRLLPVLCQDHGLTPDQVVAIASHDGGKQALESIVAQLSRPDPALAALTNDH
LVALACLGGRPALDAVKKGLPHAPALIKRTNRRIPERTSHRVAGSGGG,
which targets the binding site:
(SEQ ID NO: 66)
5′-TCTCCCGCTGACTGCGCCT-3′
TALE BCL11A #16
(SEQ ID NO: 67)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASNGGGKQALETVQRLLPVLCQDHGLTPDQVVAIASHD
GGKQALETVQRLLPVLCQDHGLTPDQVVAIASNNGGKQALETVQRLLPVLCQDHGL
TPDQVVAIASHDGGKQALETVQRLLPVLCQDHGLTPDQVVAIASNNGGKQALETVQ
RLLPVLCQDHGLTPDQVVAIASHDGGKQALETVQRLLPVLCQDHGLTPDQVVAIAS
NGGGKQALETVQRLLPVLCQDHGLTPDQVVAIASNGGGKQALETVQRLLPVLCQD
HGLTPDQVVAIASNNGGKQALETVQRLLPVLCQDHGLTPDQVVAIASHDGGKQALE
TVQRLLPVLCQDHGLTPDQVVAIASHDGGKQALETVQRLLPVLCQDHGLTPDQVVA
IASHDGGKQALETVQRLLPVLCQDHGLTPDQVVAIASNGGGKQALETVQRLLPVLC
QDHGLTPDQVVAIASHDGGKQALETVQRLLPVLCQDHGLTPDQVVAIASHDGGKQ
ALETVQRLLPVLCQDHGLTPDQVVAIASHDGGKQALESIVAQLSRPDPALAALTNDH
LVALACLGGRPALDAVKKGLPHAPALIKRTNRRIPERTSHRVAGSGGG,
which targets the binding site:
(SEQ ID NO: 68)
5′-TCCCTTGCTGCCAAACTTT-3′
TALE BCL11A #17
(SEQ ID NO: 69)
MGKPIPNPLLGLDSTGGMAPKKKRKVDGGVDLRTLGYSQQQQEKIKPKVRSTVAQ
HHEALVGHGFTHAHIVALSQHPAALGTVAVKYQDMIAALPEATHEAIVGVGKQWSG
ARALEALLTVAGELRGPPLQLDTGQLLKIAKRGGVTAVEAVHAWRNALTGAPLNLTP
DQVVAIASHDGGKQALETVQRLLPVLCQDHGLTPDQVVAIASHDGGKQALETVQRL
LPVLCQDHGLTPDQVVAIASHDGGKQALETVQRLLPVLCQDHGLTPDQVVAIASHD
GGKQALETVQRLLPVLCQDHGLTPDQVVAIASHDGGKQALETVQRLLPVLCQDHGL
TPDQVVAIASNNGGKQALETVQRLLPVLCQDHGLTPDQVVAIASNNGGKQALETVQ
RLLPVLCQDHGLTPDQVVAIASHDGGKQALETVQRLLPVLCQDHGLTPDQVVAIAS
HDGGKQALETVQRLLPVLCQDHGLTPDQVVAIASHDGGKQALETVQRLLPVLCQD
HGLTPDQVVAIASNGGGKQALETVQRLLPVLCQDHGLTPDQVVAIASNIGGKQALE
TVQRLLPVLCQDHGLTPDQVVAIASNNGGKQALETVQRLLPVLCQDHGLTPDQVVA
IASHDGGKQALETVQRLLPVLCQDHGLTPDQVVAIASNGGGKQALETVQRLLPVLC
QDHGLTPDQVVAIASHDGGKQALETVQRLLPVLCQDHGLTPDQVVAIASHDGGKQ
ALETVQRLLPVLCQDHGLTPDQVVAIASNGGGKQALESIVAQLSRPDPALAALTNDH
LVALACLGGRPALDAVKKGLPHAPALIKRTNRRIPERTSHRVAGSGGG,
which targets the binding site:
(SEQ ID NO: 70)
5′-TGGGCCCTCACGCCTTTCT-3′

Meganucleases (Silve, G. et al. (2011) Cur. Gene Ther. 11: 11-27) and CRISPR/Cas systems (Sander, J. D. et al. (2014) Nat. Biotechnol. 32: 347-55) may also be employed as suitable DNA-binding domains in the ATRs of the present invention.

The CRISPR/Cas system is an RNA-guided DNA binding system (van der Oost et al. (2014) Nat. 25 Rev. Microbiol. 12: 479-92), wherein the guide RNA (gRNA) may be selected to enable an ATR comprising a Cas9 domain to be targeted to a specific sequence. Thus, to employ the CRISPR/Cas system as a DNA-binding domain in the present invention it is to be understood that an ATR effector domain may be operably linked to a Cas9 endonuclease. Preferably, the ATR effector domain is operably linked to a Cas9 endonuclease which has been inactivated such that it substantially does not possess nuclease activity. The ATR comprising the Cas9 endonuclease may be delivered to a target cell in combination with one or more guide RNAs (gRNAs). The guide RNAs are designed to target the ATR to a target gene of interest or a regulatory element (e.g. promoter, enhancer or splicing sites) of the target gene. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390. [Epub ahead of print]; Zalatan, J. G. et al. (2015) Cell 160: 339-50; Paix, A. et al. (2015) Genetics 201: 47-54), and are suitable for use in the present invention.

For example, an ATR of the present invention may comprise the sequence:

(catalytically inactive Cas9; SEQ ID NO: 22)
MGGRRVRWEVYISRALWLTREPTAYVVLIEINTTHYRETQATGATMYPYD
VPDYASPKKKRKVEASDKKYSIGLAIGTNSVGWAVITDEYKVPSKKFKVL
GNTDRHSIKKNLIGALLFDSGETAEATRLKRTARRRYTRRKNRICYLQEI
FSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKK
GILQTVKVVDELVKVMGRHKPENIVIEMARENQTTQKGQKNSRERMKRIE
EGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYVVR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDSPKKKRKVG

The ATR of the present invention may, for example, comprise an amino acid sequence that has 50%, 60%, 70%, 80%, 90%, 95%, 99% or 100% identity to SEQ ID NO: 22 wherein the amino acid sequence substantially retains the natural function of the protein represented by SEQ ID NO: 22.

The ATR of the present invention may, for example, be encoded by a polynucleotide comprising a nucleic acid sequence which encodes the protein of SEQ ID NO: 22, or a protein that has 50%, 60%, 70%, 80%, 90%, 95%, 99% or 100% amino acid identity to SEQ ID NO: 22 wherein the amino acid sequence substantially retains the natural function of the protein represented by SEQ ID NO: 22.

The ATR of the present invention may, for example, comprise an amino acid sequence that has at least 50%, 60%, 70%, 80%, 90%, 95%, 99% or 100% identity to SEQ ID NO: 22 wherein the amino acid sequence substantially retains the natural function of the protein represented by SEQ ID NO: 22.

The ATR of the present invention may, for example, be encoded by a polynucleotide comprising a nucleic acid sequence which encodes the protein of SEQ ID NO: 22, or a protein that has at least 50%, 60%, 70%, 80%, 90%, 95%, 99% or 100% amino acid identity to SEQ ID NO: 22 wherein the amino acid sequence substantially retains the natural function of the protein represented by SEQ ID NO: 22.

Example sequences of genomic target sites recognised by guide RNAs (gRNAs) for use in targeting the β2-microglobulin gene include:

gRNA #1:
(SEQ ID NO: 23)
TATAAGTGGAGGCGTCGCGC
gRNA #2:
(SEQ ID NO: 24)
GCCCGAATGCTGTCAGCTTC
gRNA #3:
(SEQ ID NO: 25)
TGCGTCGCTGGCTTGGAGAC
gRNA #4:
(SEQ ID NO: 26)
CCAATCAGGACAAGGCCCGC
gRNA #5:
(SEQ ID NO: 27)
AGGGTAGGAGAGACTCACGC
gRNA #6:
(SEQ ID NO: 28)
GCGGGCCACCAAGGAGAACT
gRNA #7:
(SEQ ID NO: 29)
GCTACTCTCTCTTTCTGGCC
gRNA #8:
(SEQ ID NO: 30)
CTCCCGCTCTGCACCCTCTG
gRNA #9:
(SEQ ID NO: 31)
TTTGGCCTACGGCGACGGGA
gRNA #10:
(SEQ ID NO: 32)
GGGGCAAGTAGCGCGCGTCC
gRNA #11:
(SEQ ID NO: 33)
TAGTCCAGGGCTGGATCTCG

Example of guide RNAs (gRNAs) for use in targeting the β2-microglobulin gene include:

gRNA #1:
(SEQ ID NO: 149)
UAUAAGUGGAGGCGUCGCGC
gRNA #2:
(SEQ ID NO: 150)
GCCCGAAUGCUGUCAGCUUC
gRNA #3:
(SEQ ID NO: 151)
UGCGUCGCUGGCUUGGAGAC
gRNA #4:
(SEQ ID NO: 152)
CCAAUCAGGACAAGGCCCGC
gRNA #5:
(SEQ ID NO: 153)
AGGGUAGGAGAGACUCACGC
gRNA #6:
(SEQ ID NO: 154)
GCGGGCCACCAAGGAGAACU
gRNA #7:
(SEQ ID NO: 155)
GCUACUCUCUCUUUCUGGCC
gRNA #8:
(SEQ ID NO: 156)
CUCCCGCUCUGCACCCUCUG
gRNA #9:
(SEQ ID NO: 157)
UUUGGCCUACGGCGACGGGA
gRNA #10:
(SEQ ID NO: 158)
GGGGCAAGUAGCGCGCGUCC
gRNA #11:
(SEQ ID NO: 159)
UAGUCCAGGGCUGGAUCUCG

All the above RNAs may be fused to the RNA scaffold with the followinq sequence:

(SEQ ID NO: 160)
GUUUAAGAGCUAUGCUGGAAACAGCAUAGCAAGUUUAAAUAAGGCUAGUC
CGUUAUUCAACUUGAAAAAGUGGCACCGAGUCGGUGCU.

Example sequences of gRNAs targeting the BCL11A gene include:

gRNA #1 against CpG 105:
(SEQ ID NO: 71)
GCCUUUCUGCAGACGUUCCC
gRNA #2 against CpG 105:
(SEQ ID NO: 72)
UGGGUGUGCGCCUUGGCCGG
gRNA #3 against CpG 105:
(SEQ ID NO: 73)
CGGUGGUGAGAUGACCGCCU
gRNA #4 against CpG 105:
(SEQ ID NO: 74)
GGAAUGUGCUCACGGCGCCG
gRNA #5 against CpG 105:
(SEQ ID NO: 75)
GACUGCCCGCGCUUUGUCCU
gRNA #6 against CpG 105:
(SEQ ID NO: 76)
CCAGAGUCUGGCCCCCGGAG
gRNA #7 against CpG 105:
(SEQ ID NO: 77)
UCUGCGACCCUUAGGAGCCG
gRNA #8 against CpG 105:
(SEQ ID NO: 78)
GAGCGCCCCGCCAAGCGACU
gRNA #9 against CpG 105:
(SEQ ID NO: 79)
CAAGUCUCCAGGAGCCCGCG
gRNA #10 against CpG 105:
(SEQ ID NO: 80)
CGCGGAAUCCAGCCUAAGUU
gRNA #11 against CpG 105:
(SEQ ID NO: 81)
CCCGCUGCGGAGCUGUAACU
gRNA #1 against CpG 31:
(SEQ ID NO: 82)
CGCUCCUGAGUCCGCGGAGU
gRNA #2 against CpG 31:
(SEQ ID NO: 83)
CACGGCUCUCCCCGUCGCCG
gRNA #3 against CpG 31:
(SEQ ID NO: 84)
CCGCCUUUUGUUCCGGCCAG
gRNA #4 against CpG 31:
(SEQ ID NO: 85)
GCGCGAGGAGCCGGCACAAA
gRNA #5 against CpG 31:
(SEQ ID NO: 86)
GCCACUUUCUCACUAUUGUG
gRNA #6 against CpG 31:
(SEQ ID NO: 87)
GCUGCCUCUGAGGUUCGGUC
gRNA #7 against CpG 31:
(SEQ ID NO: 88)
AAGGGCAGGAGCUAGGGCCG
gRNA #8 against CpG 31:
(SEQ ID NO: 89)
GAGCCCGGACUGCUGCCUCC
gRNA #1 against CpG 38:
(SEQ ID NO: 90)
GUUUACAAGCACCGCGUGUG
gRNA #2 against CpG 38:
(SEQ ID NO: 91)
AACAGACAGAGGACCGAGCG
gRNA #3 against CpG 38:
(SEQ ID NO: 92)
GGCGCCGGGUGGGCGAUCCG
gRNA #4 against CpG 38:
(SEQ ID NO: 93)
GGUCGGGCAAGGCCCGGGCG
gRNA #5 against CpG 38:
(SEQ ID NO: 94)
AAGAGGUCUCGGCAUUGUGC
gRNA #6 against CpG 38:
(SEQ ID NO: 95)
GUUCCACAGCUUCGGGACCGCG
gRNA #7 against CpG 38:
(SEQ ID NO: 96)
GAAAUCGGCUGGGUGAAACU
gRNA #8 against CpG 38:
(SEQ ID NO: 97)
GCAGUGUCUCCGCGCCAGCC
gRNA #9 against CpG 38:
(SEQ ID NO: 98)
CCUCCCCUCCCCUCCGCCCUGGG
gRNA #1 against CpG 115:
(SEQ ID NO: 99)
UCCUCCUGUCCCGGGGUUAAAGG
gRNA #2 against CpG 115:
(SEQ ID NO: 100)
CAUCUUUUGGGACACUCUAGGCUGG
gRNA #3 against CpG 115:
(SEQ ID NO: 101)
AAGUCAGGCCCUUCUUCGGAAGG
gRNA #4 against CpG 115:
(SEQ ID NO: 102)
GCAGCCUGGACUGCGCGCCCCGG
gRNA #5 against CpG 115:
(SEQ ID NO: 103)
UGCCCGGCGAUUCUCGUCCG
gRNA #6 against CpG 115:
(SEQ ID NO: 104)
UGAGCCAUUCGGUCGCUAGG
gRNA #7 against CpG 115:
(SEQ ID NO: 105)
GGUGGUACUGAGGACCGGGA
gRNA #8 against CpG 115:
(SEQ ID NO: 107)
AUUUUCUGGGUGCUCAGAGG
gRNA #9 against CpG 115:
(SEQ ID NO: 108)
UGGUCUCAGCUCGCGCACGG
gRNA #10 against CpG 115:
(SEQ ID NO: 109)
ACAAAGACAUACGGGGUGAU

Example sequences of gRNAs targeting the IFNAR1 gene include:

gRNA #1:
(SEQ ID NO: 106)
AGGAACGGCGCGUGCGCGGA
gRNA #2:
(SEQ ID NO: 161)
AAGAGGCGGCGCGUGCGTAG
gRNA #3:
(SEQ ID NO: 162)
GGGCGGUGUGACUUAGGACG
gRNA #4:
(SEQ ID NO: 163)
CCAGAUGAUGGUCGUCCUCC
gRNA #5:
(SEQ ID NO: 164)
GACCCUAGUGCUCGUCGCCG
gRNA #6:
(SEQ ID NO: 165)
UGGGUGUUGUCCGCAGCCGC
gRNA #7:
(SEQ ID NO: 166)
ACGGGGGCGGCGAUGCUGUU
gRNA #8:
(SEQ ID NO: 167)
GACCGAAGGUUUCCCAGACU
gRNA #9:
(SEQ ID NO: 168)
GUCGGGUUUAAUCUUUGGCG
gRNA #10:
(SEQ ID NO: 169)
CGCUCCCGAGGACCCGUACA
gRNA #11:
(SEQ ID NO: 170)
CGGGUCCCACCCCCGUGAAA
gRNA #12:
(SEQ ID NO: 171)
UCAAACUCGACACAAAGCUC
gRNA #13:
(SEQ ID NO: 172)
GCGGAGCCGCGGUACUUUCC

Example sequences of gRNAs targeting the VEGFA gene include:

gRNA #1:
(SEQ ID NO: 173)
GGCGCGCGCGCUAGGUGGGA
gRNA #2:
(SEQ ID NO: 174)
AGAGAGGCUCACCGCCCACG
gRNA #3:
(SEQ ID NO: 175)
GUACGUGCGGUGACUCCGGU

All the above RNAs may be fused to the RNA scaffold with the following sequence:

(SEQ ID NO: 160)
GUUUAAGAGCUAUGCUGGAAACAGCAUAGCAAGUUUAAAUAAGGCUAGUC
CGUUAUUCAACUUGAAAAAGUGGCACCGAGUCGGUGCU.

### Target Gene Repression

By “silencing a target gene”, it is to be understood that the expression of the target gene is reduced to an extent sufficient to achieve a desired effect. The reduced expression may be sufficient to achieve a therapeutically relevant effect, such as the prevention or treatment of a disease. For example, a dysfunctional target gene which gives rise to a disease is preferably repressed to an extent that there is either no expression of the target gene, or the residual level of expression of the target gene is sufficiently low to ameliorate or prevent the disease state.

The reduced expression may be sufficient to enable investigations to be performed into the gene's function by studying cells reduced in or lacking that function.

Following administration of the two or more ATRs of the invention, the level of transcription or expression of the target gene may be reduced by, for example, at least 50%, 60%, 70%, 80%, 90%, 95%, 99% or 100% compared to the level of transcription or expression in the absence of the two or more ATRs.

Preferably, the two or more ATRs of the present invention have a synergistic effect in silencing a target gene. The two or more ATRs of the present invention may therefore demonstrate synergy, for example therapeutic synergy, when used as described herein.

For example, the two or more ATRs of the present invention may result in a synergistic increase in the fraction of a population of cells comprising the two or more ATRs that exhibits a silenced target gene, in comparison to a population of cells that lacks the two or more ATRs (e.g. comprises only one ATR or comprises a different combination of ATRs). Alternatively, or additionally, the two or more ATRs of the present invention may result in a synergistic increase in the duration that the target gene is silenced in a population of cells comprising the two or more ATRs, in comparison to a population of cells that lacks the two or more ATRs.

Preferably, the silencing of the target gene occurs following transient delivery or expression of the ATRs of the present invention to or in a cell.

By “transient expression”, it is to be understood that the expression of the ATR is not stable over a prolonged period of time. Preferably, the polynucleotide encoding the ATR does not integrate into the host genome. More specifically, transient expression may be expression which is substantially lost within 20 weeks following introduction of the polynucleotide encoding the ATR into the cell. Preferably, expression is substantially lost within 12, 6, 4 or 2 weeks following introduction of the polynucleotide encoding the ATR into the cell.

Similarly, by “transient delivery”, it is to be understood that the ATR substantially does not remain in the cell (i.e. is substantially lost by the cell) over a prolonged period of time. More specifically, transient delivery may result in the ATR being substantially lost by the cell within 20 weeks following introduction of the ATR into the cell. Preferably, the ATR is substantially lost within 12, 6, 4 or 2 weeks following introduction of the ATR into the cell.

Methods for determining the transcription of a gene, for example the target of an ATR, are known in the art. Suitable methods include reverse transcription PCR and Northern blot-based approaches. In addition to the methods for determining the transcription of a gene, methods for determining the expression of a gene are known in the art. Suitable additional methods include Western blot-based or flow cytometry approaches.

The effect of an ATR or combination of ATRs may be studied by comparing the transcription or expression of the target gene, for example a gene endogenous to a cell, in the presence and absence of the ATRs or combination of ATRs.

The effect of an ATR or combination of ATRs may also be studied using a model system wherein the expression of a reporter gene, for example a gene encoding a fluorescent protein, is monitored. Suitable methods for monitoring expression of such reporter genes include flow cytometry, fluorescence-activated cell sorting (FACS) and fluorescence microscopy.

For example, a population of cells may be transfected with a vector which harbours a reporter gene. The vector may be constructed such that the reporter gene is expressed when the vector transfects a cell. Suitable reporter genes include genes encoding fluorescent proteins, for example green, yellow, cherry, cyan or orange fluorescent proteins. In addition, the population of cells may be transfected with vectors encoding the ATRs of interest. Subsequently, the number of cells expressing and not-expressing the reporter gene, as well as the level of expression of the reporter gene may be quantified using a suitable technique, such as FACS. The level of reporter gene expression may then be compared in the presence and absence of the ATRs.

Preferably, the target gene is silenced permanently. By “permanent silencing” of a target gene, it is to be understood that transcription or expression of the target gene is reduced (e.g. reduced by 100%) compared to the level of transcription or expression in the absence of the two or more ATRs for at least 2 months, 6 months, 1 year, 2 year or the entire lifetime of the cell/organism. Preferably, a permanently silenced target gene remains silenced for the remainder of the cell's life.

Preferably the target gene remains silenced in the progeny of the cell to which the two or more ATRs of the invention has been administered (i.e. the silencing of the target gene is inherited by the cell's progeny). For example, the two or more ATRs of the invention may be administered to a stem cell (e.g. a haematopoietic stem cell) to silence a target gene in a stem cell and also in the stem cell's progeny, which may include cells that have differentiated from the stem cell.

A target gene may be silenced by using ATRs which bind to the target gene itself or to regulatory sequences for the target gene (e.g. promoter or enhancer sequences). Furthermore, alternative splicing of a target gene may be altered by using ATRs which bind to the splicing sites of the target gene itself. The ability to silence a target gene or to modulate its splicing variants by using ATRs which bind to regulatory sequences is not possible with certain other gene silencing technologies and is a particular advantage of the present invention.

### Use in Therapy

In another aspect, the present invention provides the products, artificial transcription repressors (ATRs), polynucleotides and cells of the present invention for use in therapy.

The use in therapy may, for example, be a use for the treatment of β-thalassemia or sickle cell anaemia.

The use in therapy may, for example, be a use for the preparation of “universally” allogeneic transplantable cells (e.g. by the silencing of β2-microglobulin, B2M). This use may, for example, be applied to the preparation of haematopoietic stem and/or progenitor cells (HSPCs), whole organ transplantation and cancer immunotherapy.

The two or more ATRs, or polynucleotides encoding therefor, may be administered simultaneously, in combination, sequentially or separately (as part of a dosing regime).

By “simultaneously”, it is to be understood that the two agents are administered concurrently, whereas the term “in combination” is used to mean they are administered, if not simultaneously, then “sequentially” within a time frame that they both are available to act therapeutically within the same time frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction there-between, and their respective half-lives.

In contrast to “in combination” or “sequentially”, “separately” is to be understood as meaning that the gap between administering one agent and the other agent is significant, i.e. the first administered agent may no longer be present in the bloodstream in a therapeutically effective amount when the second agent is administered.

### Target Gene

Preferably, the target gene gives rise to a therapeutic effect when silenced.

By way of example, the products, artificial transcription repressors (ATRs) and polynucleotides of the present invention may be used to silence β2-microglobulin (B2M), BCL11A, KLF1, globin genes, CCR5, CXCR4, TCR genes, miR126, PDL1, CTLA4, COL1A1, viral sequences and oncogenes.

Silencing of the TCR genes, PDL1 and CTLA4 may be used to improve efficacy of cancer immunotherapy approaches.

Silencing of B2M may be used to generate allogeneic HSPCs, T-cells or mesenchymal cells to be used for transplantation.

Silencing of miR126 may be used to expand the more primitive haematopoietic stem cell pool prior to or after their infusion.

By way of example, the products, artificial transcription repressors (ATRs), polynucleotides and cells of the present invention may be used in the treatment of, for example, Huntington's disease, Spinocerebellar ataxias, collagenopathies, haemaglobinopathies and diseases caused by trinucleotide expansions. Furthermore, the product of the present invention may be used in the treatment or prevention of certain infectious diseases (e.g. CCRS-tropic HIV infections) by inactivating either pathogen-associated gene products or host genes that are necessary for the pathogen life cycle.

In addition, or in the alternative, the products, artificial transcription repressors (ATRs), polynucleotides and cells of the present invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the products, artificial transcription repressors (ATRs), polynucleotides and cells of the present invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the products, artificial transcription repressors (ATRs), polynucleotides and cells of the present invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Proteins

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

### Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non - polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### Vectors

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides used in the invention may be introduced into cells using a variety of techniques known in the art, such as transfection, transformation and transduction.

Several such techniques are known in the art, for example infection with recombinant viral vectors, such as retroviral, lentiviral (e.g. integration-defective lentiviral), adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

The term “transfection” is to be understood as encompassing the delivery of polynucleotides to cells by both viral and non-viral delivery.

### Protein Transduction

As an alternative to the delivery of polynucleotides to cells, the products and artificial transcription repressors (ATRs) of the present invention may be delivered to cells by protein transduction.

Protein transduction may be via vector delivery (Cai, Y. et al. (2014) Elife 3: e01911; Maetzig, T. et al. (2012) Curr. Gene Ther. 12: 389-409). Vector delivery involves the engineering of viral particles (e.g. lentiviral particles) to comprise the proteins to be delivered to a cell. Accordingly, when the engineered viral particles enter a cell as part of their natural life cycle, the proteins comprised in the particles are carried into the cell.

Protein transduction may be via protein delivery (Gaj, T. et al. (2012) Nat. Methods 9: 805-7). Protein delivery may be achieved, for example, by utilising a vehicle (e.g. liposomes) or even by administering the protein itself directly to a cell.

### Pharmaceutical Composition

The products, artificial transcription repressors (ATRs), polynucleotides and cells of the present invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy products is preferably performed in compliance with FACT-JACIE International Standards for cellular therapy.

### Kit

In one aspect, the present invention provides a kit comprising two or more artificial transcription repressors (ATRs), or polynucleotides encoding therefor, selected from groups (a), (b) or (c): (a) an ATR comprising a DNA-binding domain operably linked to a KRAB domain or homologue thereof; (b) an ATR comprising a DNA-binding domain operably linked to a DNMT3A, DNMT3B or DNMT1 domain or homologue thereof; and (c) an ATR comprising a DNA-binding domain operably linked to a DNMT3L domain or homologue thereof, wherein at least two of the ATRs are selected from different groups (a), (b) or (c).

The two or more ATRs, or polynucleotides encoding therefor, may be provided in suitable containers.

The kit may also include instructions for use, for example instructions for the simultaneous, sequential or separate administration of the two or more ATRs, or polynucleotides encoding therefor, to a subject in need thereof.

### Method of Treatment

It is to be appreciated that all references herein to treatment include curative, palliative and prophylactic treatment; although in the context of the present invention references to preventing are more commonly associated with prophylactic treatment. The treatment of mammals, particularly humans, is preferred. Both human and veterinary treatments are within the scope of the present invention.

### EXAMPLES

### Example 1

With the aim of recapitulating the endogenous epigenetic mechanisms that permanently silence endogenous retroviruses (ERVs) during development, we employed the Kruppel-associated box (KRAB) domain of human zinc finger protein 10 (ZNF10; Szulc, J. et al. (2006) Nat. Methods 3: 109-16) and the catalytic domain of human DNA methyltransferase 3A (DNMT3A; Law, J. A. et al. (2010) Nat. Rev. Genet. 11: 204-20). The amino acid sequences of these domains are shown in Table 1.

To test the activity and stability of gene silencing induced by these two effector domains we used the tetracycline (tet) responsive system. We separately fused the two effector domains to the E. coli tetracycline-controlled Repressor (tetR) DNA-binding domain (Gossen, M. et al. (1992) Proc. Natl. Acad. Sci. USA 89: 5547-51), generating the tetR:KRAB and the tetR:DNMT3A artificial transcription repressors (ATRs, hereafter referred as tetR:K and tetR:D3A, respectively). The advantage of the tetR system is that it allows temporal control of binding of tetR to its target nucleotide sequence, the tetracycline operon (TetO), by doxycycline (doxy) administration. This allows us to investigate if the chromatin states induced by the ATRs can be maintained after the release of the ATRs from their target locus.

To rapidly assess activity of the ATRs we devised an experimental cell model in which activity of the ATRs can be easily followed over time by flow cytometry analyses (FIG. 1). Specifically, we generated single cell-derived clones of K562 cells engineered to contain within the first intron of the PPP1R12C gene (Lombardo, A. et al. (2011) Nat. Methods 8: 861-9; also known as the AAVS1 locus) homozygous insertion of an eGFP-expression cassette followed by seven tandem repeats of TetO (TetO7; FIG. 1, top schematic). Expression of the eGFP marker in this reporter construct is driven by the ubiquitously expressed human phosphoglycerate kinase (hPGK) gene promoter. This reporter cell line will hereafter be referred to as the AAVS1/TetO7 cell line.

Upon expression of the ATRs, these chimeric proteins bind to the TetO7 element through their tetR DNA binding domain, thus eventually leading to the deposition of repressive epigenetic marks over the nearby chromatin (shown as red lollipops on the hPGK promoter; FIG. 1, middle schematic). This induces transcriptional silencing of the cassette. Upon conditional release of the ATR from the TetO7 element by doxy administration, the repressive marks can be either erased or propagated to the cell progeny by the endogenous cell machinery, thereby leading to transcriptional reactivation or permanent silencing of eGFP expression, respectively (FIG. 1, bottom schematics). Major advantages in the use of such an experimental model are: i) activity of the ATRs can be rapidly and easily monitored by observing eGFP expression by flow cytometry analysis; and ii) because these clones were engineered to contain homozygous insertion of the cassette, we can study the epigenetic and transcriptional impact of silencing on the genes at and nearby the integration site without the confounding effect of the unmodified wild-type locus.

In order to assess if the new ATRs were biologically active, we delivered the tetR:K and tetR:D3A into the AAVS1/TetO7 cell line using standard integrating bidirectional lentiviral vectors (Amendola, M. et al. (2005) Nat. Biotechnol. 23: 108-16; Bid.LV; FIG. 2A). The advantage of these vectors is that they constitutively co-express the ATRs and a marker gene (either the truncated low-affinity nerve growth factor receptor -ΔLNGFR- or monomeric orange -mOrange-) from the same promoter, thus allowing us to restrict our analysis of silencing exclusively to the cells expressing the ATRs.

In summary, by virtue of the experimental setting described, we can test if constitutive binding of candidate ATRs to the TetO7 cassette is able to deposit repressive epigenetic marks over the nearby chromatin and to induce transcriptional silencing of the reporter cassette. In this case, the subsequent conditional release of ATR binding by doxy administration allows us to discern if the artificially induced repressive marks are then erased (thereby leading to transcriptional reactivation), or propagated to the cell progeny by the endogenous machinery (thus indicating that a permanently inherited epigenetic silencing state has been established).

Upon molecular characterisation, the AAVS1/TetO7 cell line was transduced with either Bid.LV-tetR:K or Bid.LV-tetR:D3A in the presence or absence of doxy and then maintained in these culture conditions for up to 200 days. During this time, the cells were periodically analysed by flow cytometry to measure the percentage of eGFP-negative (eGFP−) cells within the Bid.LV-transduced cell populations. As shown in FIG. 2B, constitutive binding of the ATRs to the TetO7 sequence (doxy- conditions) eventually led to eGFP silencing in 100% of the transduced cells, although with different kinetics between the two ATRs. Specifically, the tetR:K transduced cells rapidly became eGFP- (FIG. 2B, left histogram) and this effect was independent of the level of transduction (FIGS. 2C-2D, left flow cytometry dot blots). On the other hand, silencing induced by tetR:D3A was significantly slower (FIG. 2B, right histogram). In this case, the cells with the higher expression level of the marker gene (likely those with higher vector copy number, VCN) were the first to be silenced (FIGS. 2C-2D, right flow cytometry dot blots), indicating the requirement of a certain level of expression of the tetR:D3A to ensure faster repression. Importantly, at later time points (˜200 days), flow cytometry analyses showed that the mean fluorescence intensity (MFI) of eGFP was superimposable between silenced and wild-type (WT) K562 cells (compare MFIs in FIGS. 2C-2D), indicating complete silencing of eGFP expression. When doxy was present in the cultures (doxy+conditions), none of the transduced cells silenced eGFP, indicating that ATR binding to the target sequence is necessary to induce silencing. Overall, these data show that both ATRs are functional although they induce silencing with different kinetics.

We then assessed if release of the ATRs from the locus would result in eGFP reactivation. To this aim, we sorted the eGFP- cells at day 21 post Bid.LVs transduction and then cultured these cells in the presence or absence of doxy for an additional 170 days. Interestingly, doxy administration resulted in two opposite outcomes according to the ATR used: silencing induced by tetR:K was rapidly (within 15 days post doxy administration) and fully erased in the whole cell population (FIG. 2E, left histogram and representative flow cytometry analyses at the bottom); while silencing induced by tetR:D3A was maintained unaltered throughout the duration of the experiment (FIG. 2E, right histogram and representative flow cytometry analyses at the bottom). This clearly indicates that, opposite to tetR:K which has to be continuously active on the locus to repress it, tetR:D3A is able to establish repressive epigenetic modifications that can be permanently propagated by the endogenous cellular machinery even in the absence of the initial stimulus. This difference can be explained by the fact that in somatic cells, the KRAB-based machinery is not able to efficiently induce DNA methylation (which can be stably propagated), and deposits only reversible epigenetic marks, such as H3K9 methylation (Hathaway, N. A. et al. (2012) Cell 149: 1447-60).

Overall, these experiments clearly show that even in the absence of binding of tetR:D3A to the TetO7 element, silencing of the reporter cassette can be maintained unaltered throughout several cell generations. On the other hand, conditional release of tetR:K from the TetO7 element leads to rapid and full reactivation of eGFP expression in tetR:K transduced cells.

DNA Methylation is Involved in the Maintenance of Permanent Silencing Induced by tetR:D3A

In order to understand if DNA methylation was necessary to maintain the repressive state induced by tetR:D3A, the eGFP- cells from the doxy-conditions in FIG. 2E were treated with either 5-Aza-2′-deoxycytidine (5-Aza) or vehicle (i.e. Dimethyl Sulfoxide, DMSO) and then analysed by flow cytometry to measure eGFP expression. 5-Aza is a cytosine analogue that after becoming incorporated into DNA is recognised by DNA methyltransferase as a substrate, establishing a covalent bond that, contrary to cytosine, is not resolved, thus blocking DNMT activity (Issa, J. P. et al. (2005) Nat. Rev. Drug Discov. Suppl. S6-7). As shown in FIG. 2F, treatment with 5-Aza resulted in full reactivation of eGFP expression. As expected, DMSO treatment did not alter silencing of eGFP, with eGFP+ cells in the culture representing contaminant cells from the cell sorting procedure.

Contrary to tetR:K, tetR:D3A-Induced Repression is Confined to the Target Locus

One of the requisites necessary for a safe epigenetic therapy approach is that silencing should not spread into the genes surrounding the desired target gene. Note that the site-specific integration of the reporter cassette into the AAVS1 locus allows us to easily analyse the impact of our silencing platform on the expression of genes embedded near the reporter cassette integration site. Thus, we compared the expression levels of the genes at and nearby the AAVS1 integration site (FIG. 3A) between the eGFP-cells from FIGS. 2A-2F and the untreated AAVS1/TetO7 cell line.

eGFP-cells transduced with tetR:K significantly down-regulated all the analysed genes (FIG. 3B, left histogram; data are represented as mean±SEM, n=3), indicating that this ATR deposits repressive marks able to spread for at least 340 kb (˜170 kb on both sides of the ATR binding site). This finding is consistent with previous studies performed in other somatic cell lines and showing that tetR:K can silence promoters located several tens of kilobases away from the ATR binding sites through the long-range spreading of H3K9me3 (Groner, A. C. et al. (2010) PLoS Genet. 6: e1000869). Importantly, when analysing the eGFP-cells transduced with tetR:D3A and grown with doxy, only eGFP and, to a lesser extent, the PPP1R12C gene (hosting the reporter cassette in its first intron) showed a significant down-regulation (FIG. 3B, right histogram; data are represented as mean±SEM, n=3; ***p<0.0001 and **p<0.001, one-way anova and Bonferroni post-test), indicating a very localised epigenetic repression.

Overall, these experiments show that tetR:K induces a rapid and robust transcriptional repression, capable of long-range spreading, which however is reversible upon ATR release from the locus. On the other hand, tetR:D3A induces silencing with slower kinetics, but this transcriptional repression is sharply confined to the target locus and permanently maintained even in the absence of the initial stimulus

Synergistic Activity of the ATRs Upon their Transient Co-Delivery

We then asked if the transient co-delivery of these two ATRs was sufficient to induce rapid (as tetR:K) and permanent (as tetR:D3A) epigenetic silencing. To answer to this question we transfected the AAVS1/TetO7 cell line with plasmids encoding for the ATRs, either alone or in combination, and then followed eGFP expression in these cells by time course flow cytometry analysis. Representative examples of these experiments are shown in FIG. 4A, in which we report the kinetics of silencing of the eGFP-expression cassette (% of eGFP-cells; data are represented as mean±SEM, n=3) in cells transfected with the plasmids encoding for the indicated ATRs, and the corresponding flow cytometry dot plot analyses performed at termination of the experiments. From these analyses we found that: i) none of the cells transfected with the plasmid encoding for either the tetR:K or tetR:D3A became eGFP negative, although transient delivery of the tetR:K was associated with a short wave of repression that rapidly returned to control levels by day 10 post-transfection (this latter data indicates transient deposition of H3K9me3 followed by its disappearance concomitantly with the mitotic dilution of the tetR:K encoding plasmid); and ii) remarkably, up to 20% of the cells co-transfected with the plasmids encoding for the tetR:K and tetR:D3A became stably silenced. These data revealed a significant degree of synergy between the DNMT3A- and the KRAB-based repressors, and represent the first demonstration of permanent epigenetic silencing upon transient co-delivery of ATRs.

We then asked whether the silencing induced by the tetR:K/tetR:D3A combination was limited to the reporter cassette or instead spread along the AAVS1 locus, thus also affecting the genes nearby the insertion site of the reporter cassette. To answer to this question, we compared the expression profile of the genes at and nearby the AAVS1 integration site (a schematic of the locus is shown in FIG. 3A) between the eGFP-negative and the eGFP-positive populations sorted from the tetR:K/tetR:D3A treated conditions. In these analyses we found that the treatment resulted in significant silencing only of the reporter transgene (FIG. 4B; data are represented as mean±SEM, n=3; **p<0.001, one-way anova and Bonferroni post-test). These important data indicate that the tetR:K/tetR:D3A combination deposited punctuated epigenetic silencing only at the intended target gene, highlighting the safety our approach. Finally, treatment of the eGFP-negative sorted cells from tetR:K/tetR:D3A conditions with 5-Aza completely reactivated eGFP expression in these cells (FIG. 4C; data are represented as mean±SEM, n=3; ***p<0.0001, two-tailed unpaired t test), thus indicating that DNA methylation plays an important role in the maintenance of these epigenetic states of repression. Similar results were also found by transfecting the AAVS1/TetO7 reporter cell lines with in vitro transcribed mRNAs encoding for the ATRs (FIG. 4D). Remarkably, the extent of silencing measured in these experiments was ˜2-fold higher than that measured upon plasmid transfection, likely reflecting the better tolerability and the higher expression levels achieved by mRNA transfection. Gene expression analyses showed that only eGFP and the gene lodging the eGFP-reporter cassette (i.e. PPP1R12C) were down-regulated by the treatment (FIG. 4E; data are represented as mean±SEM, n=3; ***p<0.0001 and *p<0.01, one-way anova and Bonferroni post-test).

Silencing with ATR Combinations is Locus and Cell-Type Independent

Having shown that the two ATRs are capable of inducing permanent silencing even when transiently delivered to the cells, we then asked if this effect was locus independent. Indeed, the efficacy of an epigenetic therapy approach might depend on the chromatin environment in which the target locus is embedded, with theoretically some loci being more refractory to a specific repressive mechanism than others. For example, some published evidence suggests that loci enriched in H3K4 methylation may be protected from DNA methylation (Ooi, S.K. et al. (2007) Nature 448: 714-7). In line with this, endogenous epigenetic factors naturally present at the target locus or in its neighbouring regions may counteract the activity of the ATRs or restore the original physiological epigenetic profile of the target gene.

To address this question, we inserted the TetO7 sequence upstream of the hPGK promoter of an eGFP expression cassette, and then delivered this construct semi-randomly in the genome of the K562 cells by standard lentiviral vector transduction (a schematic of the provirus used is shown in FIG. 5A). We then sorted to purity the eGFP-expressing cells (hereafter referred as the TetO7. LV-reporter cell line) and transfected them with in vitro transcribed mRNAs encoding for the tetR:K or the tetR:D3A, either alone or in combination. Time-course flow cytometry analyses of these cells (FIG. 5B; data are represented as mean±SEM, n=3; ***p<0.0001, two-way anova and Bonferroni post-test) showed that: i) up to 32% of the cells transfected with the mRNA encoding for tetR:D3A progressively became eGFP-negative, reaching a plateau of repression in two weeks after transfection; ii) up to 80% of the cells transfected with the plasmid encoding for tetR:K rapidly became eGFP-negative but soon after most of these cells reactivated eGFP expression (contrary to the experiment performed with the AAVS1/TetO7 cell line, up to 19% of the cells remained eGFP negative); and iii) remarkably, up to 80% of the cells co-transfected with mRNAs encoding for the tetR:K/tetR:D3A became permanently silenced. Interestingly, even if we measured comparable efficiencies of silencing between the tetR:K and the tetR:K/tetR:D3A conditions at short term post-transfection, only the combination of the two factors resulted in high levels of permanent epigenetic silencing. Similar results were also found in U937 cells with random insertion of the TetO7/eGFP cassette (FIG. 5C). Here, however, the efficiencies of silencing for all treatment conditions were lower than those obtained in K562 cells, although the overall efficiencies of transfection between these two cell types were comparable. Unexpectedly, when we performed a similar experiment in B-lymphoblastoid cells containing random insertion of the TetO7/eGFP cassette, long-term stable silencing was observed only in the conditions treated with the tetR:D3A (FIG. 5D; data are represented as mean±SEM, n=3; ***p<0.0001, two-way anova and Bonferroni post-test). Contrary to the results of all the above experiments, silencing induced by the combination of the ATRs was transient, displaying kinetics that were superimposable to those measured under the tetR:K-treated conditions.

Overall, these results clearly demonstrate that the two ATRs cooperate in the establishment of stable states of epigenetic repression also when their target sites are randomly distributed throughout the lentiviral vector accessible genome of different cell types, thus indicating that the silencing mechanism might be locus-independent. Yet, these studies suggest that that several cell-intrinsic factors can modulate the in vivo activity of these proteins.

### Identification of Novel ATRs Able to Increase the Silencing Efficiency of Our Platform

While the above data provide the first demonstration to our knowledge of permanent epigenetic silencing upon transient expression of ATRs, they also indicate that several cell-intrinsic factors can modulate the in vivo activity of these proteins. For instance, the lower level of silencing observed in the U937 cell line and the unexpected lack of silencing activity of the ATR combination found in B-lymphoblastoid cells might be explained by the absence of a cofactor(s) involved in the silencing process or by the presence of a cell-type specific repressor(s). Because of this reason, the inclusion of another ATR in our cocktail might be useful to increase the efficiency of silencing of the KRAB/DNMT3A combination, either by obviating to the absence of a cofactor, or by allowing proper function of the repressive complex even when the ATRs are present at low concentrations. Thus, we investigated whether any alternative effector domains (or combination thereof) from chromatin remodelling enzymes involved in the establishment of permanent states of epigenetic repression could be used to increase the silencing efficiency of our ATRs. To this end, by mining the literature for known interactors of the DNMT3A or KRAB-ZFPs proteins (Chen, T. et al. (2014) Nat. Rev. Genet. 15: 93-106) and, more broadly, for molecules involved in the transcriptional control of cell fate specification and development (Schwartz, Y. B. et al. (2013) Nat. Rev. Genet. 14: 853-64), we identified the following candidates:
Euchromatic histone-lysine N-methyltransferase 2 (EHMT2 also known as G9a): a histone methyltransferase that catalyses dimethylation of histone H3 lysine-9 and recruits several histone deacetylases;
SET domain bifurcated 1 (SETDB1): a histone methyltransferase that deposits histone H3 lysine-9 di- and tri-methylation (two histone marks associated with transcriptional repression);
Chromobox protein homolog 5 (CBX5, also known as HP1α): a component of heterochromatin that recognises and binds histone H3K9me, leading to epigenetic repression;
DNA (cytosine-5)-methyltransferase 3-like (DNMT3L): a catalytically inactive DNA methyltransferase that activates DNMT3A by binding to its catalytic domain;
Enhancer of Zeste homolog 2 (EZH2): the catalytic subunit of the polycomb repressive complex 2, which methylates lysine-9 and lysine-27 of histone H3, thus creating binding sites for the canonical polycomb repressive complex 1;
Suppressor of variegation 4-20 homolog 2 (SUV420H2): a histone methyltransferase that specifically trimethylates lysine-20 of histone H4 (a specific histone mark associated with transcriptional repression at pericentric heterochromatin); and
Transducin-like enhancer protein 1 (TLE1): a chromatin-associated transcriptional co-repressor that binds to and inhibits the activity of a number of transcription factors.

We generated new ATRs containing the effector domains of these proteins and the DNA binding domain of tetR. Hereafter, the new ATRs will be referred as: tetR:SET (SETDB1); tetR:H (HP1-α); tetR:T (TLE1); tetR:GS or tetR:GL (according to the length of the effector domain cloned from G9a); tetR:ES or tetR:EL (according to length of the effector domain cloned from EZH2); tetR:D3L (DNMT3L); and tetR:SUV (SUV420H2). The amino acid sequences of the effector domains are listed in Table 1.

We initially tested the activity of these novel ATRs in the LV/TetO7 K562 reporter cell line by using standard integrating Bid.LV and found that, among the new ATRs, tetR:SET, tetR:GS and tetR:H efficiently induced silencing when individually and stably expressed (FIG. 6A; data are represented as mean±SEM, n=3). However, none of these ATRs reached the silencing efficiency of tetR:K and tetR:D3A. Unlike tetR:GS, tetR:GL was not efficient in this experimental setting, suggesting that inclusion of the ankyrin repeats in this longer version of G9A was negatively impacting the silencing efficiency. The inefficiency of tetR:T, tetR:SUV, tetR:ES and tetR:EL in this experimental setting may be due to the intrinsic biological inactivity of the chosen domains or to the absence in this cell line of endogenous interactors necessary for the activity of these proteins. Furthermore, we noticed a decrease in the percentage of transduced cells over time for some of the ATRs used (FIG. 6B; data are represented as mean±SEM, n=3). This data indicates a growth disadvantage of cells stably expressing the ATRs, thus strengthening the rationale of using transient delivery approaches to safely express the ATRs.

We then assessed if silencing induced by the new ATRs could be maintained even in the absence of the ATRs on the target locus. To this aim, 18 days after Bid.LV-ATR transduction, we treated the samples with doxy, and monitored eGFP expression by flow cytometry analysis (FIG. 6C; data are represented as mean±SEM, n=3). As expected, tetR:D3A-induced silencing was maintained after doxy administration. Considering the other samples, silencing was maintained only in a fraction of the originally repressed cells, which varied among the different ATRs. Particularly, tetR:K-induced silencing resulted to be more stable than the others, being maintained in up to 45.8% of the originally repressed cells. This is in contrast with that observed using the AAVS1/TetO7 K562 reporter cell line, in which 7 days post doxy administration eGFP was fully reactivated in all the transduced cells. This data indicate a role in that positioning of the TetO7 relative to the hPGK promoter and/or the epigenetic environments in which the cassette is integrated might play an important role in the maintenance of the tetR:K-induced repressive state.

We then tested the efficiency of the ATRs upon their transient delivery in the same LV/TetO7 K562 reporter cell line. Particularly, we tested the ATRs either individually (FIG. 7A) or in combination with tetR:D3A (FIG. 7B), tetR:K (FIG. 7C) or with the tetR:K+tetR:D3A combination (FIG. 7D). To better appreciate any eventual increase in the silencing efficiency above the levels measured in positive controls, these experiments were performed using non-saturating doses of the ATR-expressing plasmids. tetR:T was not tested in this experiment, since it was not available in the same plasmid backbone as the other ATRs at the time of the experiment.

By following eGFP expression in the treated cells over time by flow cytometry, we found that when individually expressed, none of the ATRs efficiently induced silencing, with tetR:K, tetR:D3A and tetR:SET repressing only up to 1% of the cell (FIG. 7A; data are represented as mean±SEM, n=3). When combined to tetR:D3A (FIG. 7B; data are represented as mean±SEM, n=3), all the new ATRs conferred a gain in silencing efficiency. However, an efficiency similar to that measured with the tetR:K+tetR:D3A condition was achieved only when tetR:D3A was combined with either tetR:SET or tetR:D3L. Furthermore, when co-delivered with tetR:K (FIG. 7C; data are represented as mean±SEM, n=3), only tetR:D3L among the new ATRs synergised better than tetR:D3A. Finally, when adding one of the new ATRs to the tetR:K+tetR:D3A combination (FIG. 7D; data are represented as mean±SEM, n=3), most of the ATRs increased silencing efficiency, thus indicating a biological activity also for those ATRs that were not working when stably, but individually, delivered (FIG. 6A). Importantly, this experiment identified the tetR:K+tetR:D3A+tetR:D3L combination as the best-performing combination, showing a striking efficiency considering the low plasmid doses employed in these experiments. Specifically, the tetR:K+tetR:D3A+tetR:D3L combination resulted in a 4.1-fold increase in silencing efficiency compared to the tetR:K+tetR:D3A combination (FIG. 7E; data are represented as mean±SEM, n=3; ***p<0.0001, one-way anova and Bonferroni post-test). Given the increment in silencing efficiency compared to both the tetR:D3A+tetR:D3L, tetR:K+tetR:D3A and tetR:D3L+tetR:K combinations, all the three ATRs play a relevant role in the tetR:K+tetR:D3A+tetR:D3L cocktail. Interestingly, starting from the evidence that tetR:SET was able to synergise with both tetR:D3A and tetR:D3A+tetR:K (see FIG. 7E), we reloaded a similar experiment at even lower ATR doses, and found that tetR:SET was also able to significantly synergise with the tetR:D3A+tetR:D3L combination (FIG. 7F; tetR:D3L shown as tetR:L). This data indicates that the tetR:D3A+tetR:D3L+tetR:SET combination can be a valid alternative to the tetR:D3A+tetR:D3L+tetR:K combination, even if with lower silencing efficiency.

Inclusion of tetR:D3L to the tetR:K+tetR:D3A Combination Allows Rescue of Silencing Efficiency in Refractory Cell Types

We then asked if the use of the tetR:K+tetR:D3A+tetR:D3L combination was able to overcome the block observed in B-lymphoblastoid cells (see FIG. 5D). To address this question, the TetO7. LV-reporter B-lymphoblastoid cell line was transfected with in vitro transcribed mRNAs encoding for the three ATRs, either alone or in different combinations (FIG. 7G; tetR:D3A shown as tetR:D; tetR:D3L shown as tetR:L; data are represented as mean±SEM, n=3). As expected from previous experiments, tetR:K+tetR:D3A co-delivery resulted in a transient wave of silencing that was completely erased after dilution of the transfected mRNAs, resulting in the absence of eGFP negative cells. However, both tetR:D3A+tetR:D3L and tetR:D3L+tetR:K were capable of inducing high levels of silencing (50% and 60%, respectively). These levels are substantially higher than those observed in conditions in which the ATRs were delivered alone (14% for tetR:D3A, and levels comparable to untreated samples for tetR:K and tetR:D3L transfected cells). Strikingly, when the three ATRs were delivered together, most of the cells became eGFP negative (up to 80%), clearly demonstrating that the addition of one single factor to the tetR:D3A/tetR:K mix was sufficient to restore silencing induction and maintenance in previously refractory cell lines. Based on these promising results, we also asked if our silencing platform could be effective in experimentally relevant cell types derived from other organisms, such as mice. To answer this question, we first transduced the murine NIH/3T3 cell line with the TetO7. LV, sorted the cells to obtain a pure eGFP-positive population (hosting on average 1 copy of vector per cell), and finally transfected them with mRNAs encoding for the tetR-based ATRs, which were delivered individually or in combination. Remarkably, flow cytometry analysis of the treated cells showed effective and long-term silencing also in this cell model: a single administration of the tetR:D3A+tetR:D3L or of the triple ATR combination led to 45% or 80% gene silencing efficiency, respectively (FIG. 7H). On the other hand, the tetR:D3A+tetR:K combination did not work, as previously observed in B-lymphoblastoid cells.

Effective Silencing by Transient Co-Delivery of ATRs Equipped with Custom-Made DNA Binding Domains

The main objective of this project was to develop an epigenetic therapy platform that can be used to silence expression of any gene of interest. Although we have already identified effector domains that when fused to the tetR synergistically cooperate to silence the promoter nearby the TetO7 element, the artificial nature of the prokaryotic TetO7/tetR system hinders therapeutic application of this technology. Moreover, the TetO7 element can accommodate with high avidity 7 tetR dimers, thus leading to stochastic homo- or hetero-dimerisation of the ATRs on this element. This occurrence may favour mutual positive interactions between repressors. For these reasons, several questions remain to be addressed in order to translate the findings obtained with the TetO7/tetR system to a situation in which each of the ATRs has single and yet independent binding site on the target gene. In particular, it is unknown whether one element (defined as a given genomic sequence containing the binding site for each of the repressors, hereafter referred as the “Silencing Element”) would be sufficient to silence a gene of interest. Furthermore, the relative order and the orientation in which the two repressors are arranged on the Silencing Element, and the distance between their binding sites might represent important determinants for the activity of the repressive complex. Of note, it is impossible to define these determinants based on the literature or by empirically testing them on an endogenous gene, as it would require designing several different ATRs each with its own binding site and affinity.

To address these questions, we developed an ad hoc engineered cell model that easily reports the silencing activity of ATRs containing transcription-activator like effector (TALE; Gaj, T. et al. (2013) Trends Biotechnol. 31: 397-405) DNA-binding domains. In this set of experiments we initially tested ATRs corresponding to the tetR:K+tetR:D3A combination.

Briefly, we fused the KRAB and DNMT3A domains to the DNA binding domains of two TALEs that recognise two different genomic target sites with high efficiency (the amino acid sequences of the two TALEs are listed in Table 2). Using this approach we obtained two TALE:KRAB fusion proteins (hereafter referred as TALE:K) and two TALE:DNMT3A fusion proteins (hereafter referred as TALE:D3A) corresponding to each of the two genomic target sites. In parallel, we inserted the two TALE target sites, spaced by progressively longer nucleotide sequences (5, 10, 15, 20, 25 and 30 bp), upstream of the hPGK promoter of an eGFP expression cassette, and then delivered these constructs semi-randomly in the genome of the K562 cell line by standard lentiviral vector transduction. Of note, the target sites for the two TALEs were placed in such a way that binding of the TALE-repressors occurs in head-to-tail (H-T) configuration. A schematic representation of these vectors is shown in FIG. 8A (on the left is depicted the vector containing the binding sites for the TALE:K→TALE:D3A configuration; on the right is depicted the vector containing the binding sites for the TALE:D3A→TALE:K configuration. We then sorted to purity the eGFP expressing cells and transfected these lines with in vitro transcribed mRNAs encoding for the TALE:K or the TALE:D3A, either alone or in combination. The cells were then analysed by time-course flow cytometry to measure the extent and duration of silencing. Representative examples of these analyses can be seen in FIGS. 8A-8C, in which we report the silencing efficiencies (% of eGFP-negative cells) of the indicated ATRs with respect to the length of the spacers (FIG. 8B; data are represented as mean±SEM, n=3), and the kinetics of silencing of the eGFP-expression cassette measured in the cell line with the 25 bp spacer (FIG. 8C; data are represented as mean±SEM, n=3; ***p<0.0001 and **p<0.001, two-way anova and Bonferroni post-test).

From these experiments we found that: i) co-delivery of TALE:D3A and TALE:K resulted in full silencing of eGFP-expression cassette in up to 25% of the treated cells; ii) the relative order of binding of the two ATRs on the target locus impacted on the overall silencing efficiency, with the TALE:D3A→TALE:K configuration performing from 2.2 to 5.4-fold better than the opposite one; iii) among the spacer lengths tested, the 25 and the 30 bp performed better than the others; and iv) individual delivery of TALE:K or TALE:D3A resulted in low (3%) or absent silencing of the eGFP-expression cassette, respectively.

Considering the significant impact of structural variables (such as spacer length and the relative order of binding of the two ATRs on the target sequence) on the silencing efficiency, we then asked if moving to a head-to-head (H-H) configuration in which the C-termini of the two ATRs face each other could be beneficial for our strategy. To move from the head-to-tail to the head-to-head configuration, starting from the reporter cassette described in FIGS. 8A-8C we maintained the 5′ TALE binding site unaltered, while we changed the orientation of the 3′ TALE binding site. This simple change allowed us to use the same four ATRs employed in the previous experiments. We also generated six eGFP reporter cassettes differing in spacer length between the two TALE target sites (5, 10, 15, 20, 25 and 30 bp) and we delivered these constructs to K562 cells via lentiviral vector transduction (a schematic of these vectors is shown in FIG. 9A). Transduced cells were then sorted to obtain pure eGFP+ populations and electroporated with plasmids encoding for TALE:K or TALE:D3A, either alone or in combination. Treated cells were then analysed by time-course flow cytometry to measure the extent and duration of silencing. To stringently compare the head-to-head to the head-to-tail configuration, we included the cell line containing the 25 bp spacer and the H-T, TALE:D3A→TALE:K configuration described in FIG. 8C in this experiment. The results of these experiments indicated that: i) co-delivery of TALE:K and TALE:D3A resulted in evident synergy even in the H-H configuration, allowing long-term silencing of the reporter cassette in up to 34.7% of the treated cells (FIG. 9B; data are represented as mean±SEM, n=3); ii) individual delivery of TALE:K or TALE:D3A resulted in low (up to 7.1%) or absent permanent silencing, respectively; and iii) the relative order of binding of the two ATRs on the target locus impacted on the overall silencing efficiency, with the TALE:D3A→TALE:K configuration performing from 1.3 to 1.7 fold better than the opposite one (FIG. 9C; data are represented as mean±SEM, n=3). However, the relative order of binding seems to have a greater impact on silencing efficiency in the head-to-tail configuration than in the head-to-head configuration (compare FIGS. 8A-8C with FIGS. 9A-9C). A bell-shaped trend seems to describe the impact of the tested spacer lengths on silencing efficiency of the H-H configuration, with the 15 bp spacer performing best both in the TALE:D3A→TALE:K and in the TALE:K→TALE:D3A configurations (even outperforming the 25 bp spacer in the head-to-tail experiment). However, the difference between the 15 bp head-to-head configuration and the 25 bp head-to-tail configuration was minimal (34.7% versus 26.8% long-term eGFP-cells, respectively, i.e. a 1.3-fold increase).

Overall, these data show for the first time to our knowledge the feasibility of achieving permanent epigenetic silencing of a desired target gene upon transient delivery of a combination of ATRs equipped with custom-made DNA binding domains. Moreover, from these studies we were able to define rules for the selection of TALE binding sites that can be used for the identification of Silencing Elements on a desired target gene. By targeting multiple Silencing Elements on the regulatory sequence of this gene we should be able to increase the efficiency of silencing.

In parallel to these studies we developed bipartite ATRs by coupling two effector domains on the same TALE, i.e. the KRAB domain at the N terminus and the DNMT3A domain at the C terminus of the TALEs (FIG. 10A). Even if transient transfection of the individual proteins was not sufficient to induce appreciable levels of gene silencing, their combination was sufficient to silence eGFP in up to 7% of the treated cells (FIG. 10B; data are represented as mean±SEM, n=3). The advantages provided by such an approach are that multiple effector domains can be delivered to the same target site while reducing the number of different mRNAs required to be produced and transfected.

### Permanent Epigenetic Silencing in Human HSPCs by Using Different Combinations of ATRs

Primary haematopoietic stem cells (HSPCs) are a clinically relevant human cell type for most of the ex vivo gene therapy applications (Biffi, A. et al. (2013) Science 341: 1233158; Aiuti, A. et al. (2013) Science 341: 1233151; Aiuti, A. et al. (2009) N. Engl. J. Med. 360, 447-458; Cartier, N. et al. (2009) Science 326: 818-23; Hacein-Bey-Abina, S. et al. (2010) N. Engl. J. Med. 363: 355-64; Cavazzana-Calvo, M. et al. (2010) Nature 467: 318-22) due to their life-long self-renewal capacity and multilineage differentiation potential. HSPC differentiation is accompanied by global chromatin remodelling, which results in a progressive transition from an open chromatin configuration to a more compacted and repressive one. As such, this cell type represents the most appropriate and stringent model to test efficacy and prove stability of our epigenetic platform. To assess if the delivery of various ATR combinations was sufficient to induce significant levels of silencing in human HSPCs, we transduced human cord blood-derived CD34+ cells from healthy individuals with the TetO7/eGFP-reporter LV described in FIG. 5A. We then transfected the cells with in vitro transcribed mRNAs encoding for tetR:D3A, tetR:K or tetR:D3L, either alone or in combinations. Transfected and un-transfected cells were then grown in liquid culture for 2 weeks in myeloid-differentiation conditions or plated in semi-solid media for a Colony Forming Unit-Cells (CFU-C) assay (for the layout of these experiments refer to FIG. 11A).

Flow cytometry analyses of the cells grown in liquid culture showed that treatment with the tetR:K resulted in a transient wave of eGFP repression that was then maintained in up to 20% of the treated cells until the end of the experiment (FIG. 11B; data are represented as mean±SEM, n=3). A similar phenotype was observed in CD34+ cells transfected with mRNA encoding for tetR:D3A and tetR:D3L. Treatment with tetR:K/tetR:D3A combination or with tetR:D3A/tetR:D3L combination resulted in a cooperative effect, showing that up to 40% of the treated cells fully silenced eGFP expression. Strikingly, by combining tetR:D3L/tetR:K or tetR:D3L/tetR:K/tetR:D3A we reached up to 90% of silencing of the reporter gene. Importantly, similar levels of silencing were observed in erythroid and myeloid cells originating in the CFU-C assay (FIG. 11C; data are represented as mean±SEM, n=3), thus indicating that silencing was maintained even upon HSPC differentiation.

### Permanent Epigenetic Silencing in Human T Lymphocytes Using Different Combinations of ATRs

To assess if the delivery of various ATR combinations was sufficient to induce significant levels of silencing in human T lymphocytes, a clinically relevant cell type for many cell-based gene therapy applications including cancer immunotherapy, we transduced human T cells from healthy individuals with the TetO7/eGFP-reporter LV described in FIG. 5A. We then transfected the cells with in vitro transcribed mRNAs encoding for tetR:D3A, tetR:K or tetR:D3L, either alone or in various combinations. Transfected and un-transfected cells were then kept in liquid culture for 3 weeks in media enriched with IL-15 and IL-7 before reactivation (for the layout of these experiments, refer to FIG. 12A).

Flow cytometry analyses of the cells showed that treatment with individual ATRs and tetR:D3A/tetR:K resulted in no or transient eGFP repression. On the other hand treatment with all the other possible ATR combinations resulted in permanent silencing of the reporter gene. Importantly, the levels of silencing measured during the initial phase of cell proliferation and in the resting phase were super-imposable, indicating that silencing is maintained even after the transcriptional and metabolic states of the cells have changed (FIG. 12B; data are represented as mean±SEM, n=3).

### Permanent Epigenetic Silencing of a Human Endogenous Gene Using Custom-Made ATRs

In order to assess if the results obtained with the eGFP reporter system could also be translated to an endogenous gene embedded in its natural epigenetic context, we generated custom-made TALEs targeting the promoter region of the 32-Microglobulin (B2M) gene (the amino acid sequences of these TALEs and the nucleotide sequences of their corresponding binding sites are listed in Table 3), and fused these TALEs to the KRAB, DNMT3A and DNMT3L effector domains (for a schematic of the system refer to FIG. 13A). The spacer length between the first and the second, or between the second and the third TALE is 1 or 20 bp, respectively. We then co-transfected HEK-293T cells with the plasmids encoding for these novel ATRs and analysed the cells by flow cytometry for B2M expression.

At 50 days post-transfection, when the percentage of B2M-negative cells was stable, we measured a significant fraction of B2M-negative cells only in the conditions treated with the TALE:D3A+TALE:D3L and the TALE:D3A+TALE:D3L+TALE:K combinations (FIG. 13B; data are represented as mean±SEM, n=3; ***p<0.0001, one-way anova and Bonferroni post-test). Remarkably, up to 80% of the cells treated with all the ATRs permanently lost B2M surface expression. In parallel experiments, we sorted the B2M-negative and the B2M-positive cells, and analysed them for surface expression of MHC-I molecules, which require B2M to be presented on the plasma membrane. We found that, contrary to the B2M-positive cells, nearly all the B2M-negative cells were also negative for MHC-I expression (FIG. 13C). We also performed on the B2M-negative and B2M-positive sorted cells gene expression analysis and found that the negative cells expressed ˜100-fold less B2M that the positive cells (FIG. 13D; data are represented as mean±SEM, n=3). Then we assessed if the three effector domains were capable of inducing permanent epigenetic silencing also when targeted to the B2M gene via the RNA-guided CRISPR/Cas9 system. To this aim, we fused in frame to a catalytically dead Cas9 (D10A+H840A; dCas9; amino acid sequence listed in Table 4) the KRAB, the DNMT3A or the DNMT3L effector domains (FIG. 13E; top drawings), and designed 11 guide RNAs (gRNAs; nucleotide sequences listed in Table 4) targeting the promoter region of the B2M gene (FIG. 13E; bottom schematic, arrows indicate the location of the CRISPR/dCas9 target sites). We then co-transfected HEK-293T cells with plasmids expressing the 11 B2M gRNAs together with all the possible combinations of the plasmids encoding for the dCas9 fusion proteins. Flow cytometry analysis of the treated HEK-293T cells 33 days after transfection showed that only the dCas9:K+dCas9:D3L, dCas9:D3A+dCas9:D3L and dCas9:K+dCas9:D3a+dCas9:D3L combinations were able to induce silencing of the B2M gene (FIG. 13F; data are represented as mean±SEM, n=3). We then assessed if B2M silencing was resistant to IFN-γ treatment, a potent inducer of B2M expression (Vraetz, T. et al. (1999) Nephrol. Dial. Transplant. 14: 2137-43; Gobin, S. J. et al. (2003) Blood 101: 3058-64). For this experiment, we used wild-type and B2M-negative cells, the latter being sorted from the triple ATR treated conditions described in FIG. 13B and FIG. 13F. As expected, IFN-γ treatment caused a significant upregulation in the expression of the 2′-5′-oligoadenylate synthetase 1 (OAS1) gene (>100-fold) in all cell types tested (FIG. 13G). On the other hand, while wild-type cells significantly upregulated B2M expression upon IFN-γ treatment both at the transcriptional and at the protein level, no increase in the expression of this gene was measured in the B2M-negative cells (FIG. 13G and FIG. 13H, respectively).

In order to assess if silencing induced by our ATRs was associated with the deposition of repressive epigenetic marks on the targeted gene, we analysed the epigenetic state of the B2M gene in wild-type and silenced cells. To this aim, we sorted to purity the cells treated with plasmid encoding for the triple TALE:ATR combination in order to obtain a pure population of silenced cells (FIG. 14A, showing representative FACS dot plot). Chromatin Immunoprecipitation (ChIP) followed by quantitative PCR analysis for the RNA polymerase II (RNA Polll) on the promoter region and the gene body of B2M showed complete absence of this protein in silenced cells, while it was highly enriched at the promoter region of untreated cells (the PPP1R12C and the CCR5 gene were used as positive or negative controls for these experiments, respectively; FIG. 14B). We also performed bisulfite analysis of B2M CpG island and found that in untreated cells the promoter region was almost deprived of 5mC at the level of the CpGs (less than 1%), while the same region in silenced cells was highly decorated with de novo DNA methylation (more than 80% on average) (FIG. 14C). DNA methylation was also responsible for silencing maintenance as AZA treatment was associated to re-expression of the B2M gene in previously silenced cells (FIG. 14D). Finally, in order to address if silencing was confined to the B2M gene, we performed transcriptional analysis of the B2M locus by RT-qPCR (FIG. 14E top schematic), and found that the only gene that was down-regulated upon transient delivery of the triple ATR combination was B2M, while expression of its neighbouring gene was unaffected (FIG. 14E).

In parallel to these experiments, we also tested silencing of the B2M gene in K-562 cells. Because this cell line does not express the MHC-I, which is strictly required for B2M surface expression, we targeted the coding sequence of the fluorescent marker tdTomato into the first intron of the B2M gene in order to faithfully report for the B2M transcriptional state (FIG. 15A). After gene targeting by CRISPR/Cas9, the tdTomato positive cells were sorted and then electroporated with plasmids encoding for either TALE- or CRISPR/dCas9-based ATRs against the B2M promoter/enhancer (the target sequences of these ATRs are the same as those of FIG. 13). Concerning the TALE-based ATRs, we found that both the TALE:D3L+TALE:K and the TALE:D3A+TALE:D3L+TALE:K combinations were able to stably silence B2M expression, with the triple ATR combination being the best performing (FIG. 15B). Condon-optimisation of the effector domains of the TALE-based ATRs improved silencing efficiency of the above-mentioned combinations (FIG. 15B; compare the red versus the green bars). Concerning the silencing activity of the CRISPR/dCas9-based ATRs, we found that all but the dCas9:D3A+dCas9:K combination was able to induce high silencing efficiency of the B2M gene (up to 55% of stably silenced cells; FIG. 15C). Finally, targeting of dCas9 fused to the catalytic domain of the TET1 enzyme (which is known to demethylate DNA; Maeder, M. L. et al. (2013) Nat. Biotechnol. 31: 1137-42) to the B2M promoter/enhancer of sorted silenced cells resulted in reactivation of the expression of this gene (FIG. 15D), further corroborating the notion that silencing induced by the triple ATR combination is dependent on DNA methylation.

To assess if B2M silencing could also be effective in primary human T lymphocytes, we electroporated human T cells from a healthy donor with in vitro transcribed mRNAs encoding for the TALE:K+TALE:D3A+TALE:D3L ATRs described above. Transfected and un-transfected cells were then kept in liquid culture for 2 weeks in media enriched with IL-15 and IL-7 (experimental scheme in FIG. 16A). Flow cytometry analyses of the cells showed that treatment with the TALE:K+TALE:D3A+TALE:D3L ATRs resulted in silencing of the B2M gene (kinetic of silencing in FIG. 16B, FACS plots in FIG. 16C).

Intriguingly, functional deconvolution of 7 gRNAs into quartets until individual singlets showed that even one gRNA was sufficient to drive efficient silencing of B2M with both the triple and the dCas9:D3A+dCas9:D3L combination (FIGS. 17A-17B). Unexpectedly, some of the single gRNA were able to induce silencing efficiencies that were comparable to those measured in the 7 gRNA pool. Furthermore, in several instances, we observed that the triple ATR combination was performing better than the dCas9:D3A+dCas9:D3L combination. Altogether, these data indicates that even one well properly positioned gRNA tethering the three ATRs on the target gene can induce its efficient silencing.

Similarly, we also investigated if a single TALE protein was sufficient to induce efficient and permanent epigenetic silencing. To this aim we generated four TALE proteins, to each of which we fused the three different effector domains, namely KRAB, DNMT3A and DNMT3L (FIG. 17B; schematic on the left). As a model we used the TALE proteins targeting B2M gene and the K562 B2M tdTomato reporter cell line previously described (FIG. 13A and FIG. 15A, respectively). Unexpectedly, also in the conditions in which the repressive domains were competing for the same binding site on the B2M gene, we obtained efficient and permanent gene silencing of the B2M gene (FIG. 17B, grey bars in the histogram). The different degree of efficiency was most likely reflecting the different binding affinity of the TALEs, with some of these working as efficiently as the control condition in which each effector domain was fused to a different DNA-binding domain (FIG. 17B; dark blue bar in the histogram).

Overall, these data show for the first time to our knowledge permanent silencing of an endogenous gene in human cells using custom made ATRs. Importantly, silencing was fully resistant to external stimuli impinging on the B2M promoter/enhancer, thus providing another line of evidence of the stability of the epigenetic modifications deposed by the triple ATRs combination. Moreover, we provide evidence of the broad applicability of our strategy by tethering the repressor domains to the endogenous gene by means of two different DNA binding technologies, namely TALE and CRISPR/Cas9.

### Transient Expression of an Un-Targeted DNMT3L Improves and Rescues Silencing Efficiency of the DNMT3A+KRAB Based ATRs in Refractory Cell Types

In order to reduce the number of different ATRs to design and construct, we investigated if at least one of the effector domains can be delivered to the cells without a DNA binding domain, and still be able to effectively cooperate with the other two ATRs targeted on the desired gene of interest. To assess if delivery of an un-targeted DNMT3L (hereafter referred to as D3L) might be as effective as its targeted counterpart in cooperating with the other two effector domains (specifically DNMT3A and KRAB), we initially took advantage of the TetO7/tetR system. We thus transfected the TetO7. LV-reporter B-lymphoblastoid cells with in vitro transcribed mRNAs encoding for the tetR-based ATRs and for the un-targeted D3L, and measured by time-course flow cytometry analysis the percentage of eGFP-negative cells in the different transfection conditions (FIG. 18A; data are represented as mean±range, n=2). At 27 days post-transfection we found little if any silencing in cells treated with either the individual ATRs or the tetR:K+tetR:D3A combination. Instead, up to 70% of the cells treated with the combination of the 3 ATRs become eGFP-negative. The targeted tetR:D3L synergised also with tetR:K or tetR:D3A, although the levels of silencing measured in these two experimental conditions were 3.5-fold lower (˜20% eGFP-negative cells) than those measured with the triple ATR combination (FIG. 18A; compare the plus tetR:D3L conditions). These data are in line with those previously found with the TetO7. LV-reporter B-lymphoblastoid cell line, in which the unexpected drop in the silencing efficiency of the tetR:D3A+tetR:K combination was completely rescued by inclusion of the tetR:D3L to the cocktail (see FIG. 7G for comparison). When the un-targeted D3L was delivered either alone or in combination with the tetR:K, no eGFP-negative cells were found. On the other hand, D3L was able to effectively synergise with both the tetR:D3A and the tetR:D3A+tetR:K combination (FIG. 18A; see the plus D3L conditions). Importantly, the levels of silencing measured in these two experimental conditions were comparable to those found by co-tethering DNMT3L and DNMT3A; or DNMT3L, DNMT3A and KRAB to the TetO7 sequence. These data indicate that the un-targeted D3L can effectively synergise with the KRAB+DNMT3A combination.

We then assessed if these findings also held true with ATRs based on custom-made DNA binding domains. To this end, we selected 4 different TALE binding sites in the B2M promoter region and constructed the corresponding TALE DNA binding domains (a schematic of the B2M locus showing the different TALEs binding sites is depicted in FIG. 18B; the amino acid sequences of the TALE A and the nucleotide sequences of its corresponding binding sites is listed in Table 5; TALEs B, C and D were described previously and correspond to TALE #1, #2 and #3 of Table 3). Each of these TALEs were equipped with KRAB or DNMT3A. The 4 different TALE binding sites constitute two independent silencing modules (Module 1: site A plus site B; Module 2: site C plus site D), at which the TALE:D3A and TALE:K can bind in two different orders (siteA:K-siteB:D3A or siteA:D3A-siteB:K). We then transfected HEK-293T cells with plasmids encoding for the TALE-based ATRs and for the untargeted D3L, and measured by time-course flow cytometry analysis the percentage of double B2M/MHCI-negative cells in the different transfection conditions (FIG. 18C). At 12 days post-transfection we measured a low fraction of B2M/MHCI-negative cells in all the conditions treated with the TALE:D3A+TALE:K combination (upper plots in FIG. 18C). On the other hand, co-treatment of the cells with the combination of the two ATRs plus the untargeted D3L resulted on average in a 5-fold increase in the efficiency of silencing (lower plots FIG. 18C) over the levels measured in the absence of D3L. This increase was independent of the relative order of binding of the TALE proteins on the B2M promoter and was confirmed for both of the silencing modules.

Finally, we performed similar experiments using ATRs based on the CRISPR/Cas9 system (FIG. 18D; data are represented as mean±SEM, n=3). Here, we found that transient expression of D3L in HEK-293T cells transfected with the dCas9:K+dCas9:D3A ATRs plus the B2M gRNAs (those used in FIG. 13E) resulted in levels of gene silencing comparable to those obtained with the triple combination of the dCas9-based ATRs plus the B2M gRNAs (FIG. 18D). Similar results were obtained by delivering D3L with the DNMT3A ATR.

Overall, these data clearly show that the un-targeted DNMT3L can effectively replace its targeted counterpart in our cocktail of ATRs.

### Transient Expression of an Untargeted DNMT3B Rescues Silencing Efficiency of the DNMT3A+KRAB Based ATRs in Refractory Cell Types

Considering the role of the DNMT3B in the establishment of de novo DNA methylation, we asked if the endogenous DNMT3B could cooperate with our ATRs. To answer this question, we performed a genetic knock-out of DNMT3B by CRISPR/Cas9 in the TetO7. LV K562 reporter cell line. To do this, we transduced the cells with two lentiviral vectors, one encoding for a doxycycline-inducible Cas9 nuclease (Wang, T. et al. (2014) Science 343: 80-4) and another encoding for both a gRNA against the exon 2 of the DNMT3B gene and the ΔLNGFR marker (schematic of the vectors in FIG. 19A; middle FACS plot for the double transduced cells). Upon Cas9 activation by doxycycline administration, we electroporated the cells with plasmids encoding for the different combinations of the ATRs, and then measured by flow cytometry the efficiency of silencing in the ΔLNGFR-positive and -negative cells. By comparing these numbers, we can appreciate if inactivation of the DNMT3B gene improves or not the efficiency of silencing of the different ATR combination. Here, we observed that the subpopulation expressing the gRNAs anti-DNMT3B (i.e. ΔLNGFR-positive cells) was less permissive than wild-type cells (i.e. those negative for ΔLNGFR) to silencing by tetRK+tetR:D3A combination (FIGS. 19B-19D; left FACS plot of FIG. 19C), thus indicating that the endogenous DNMT3B is a relevant partner of these two ATRs. Remarkably, genetic knock-out of DNMT3B increased silencing efficiency of the tetR:K+tetR:D3A+tetR:D3L combination, thus suggesting that in this case DNMT3B is acting as a decoy for these ATRs (FIGS. 19B-19D; right FACS plot of FIG. 19C). For all the other ATR combinations and the individual ATR, inactivation of DNMT3B did not cause any significant difference in the silencing efficiency as compared to wild-type cells. Furthermore, considering that, in contrast to K562 cells, the B-lymphoblastoid cell line described above lacks DNMT3B expression (as measured by RT-qPCR analysis), we asked if DNMT3B overexpression could increase ATRs silencing efficiency in this cell line refractory to the DNMT3A+KRAB combination. In particular, we transiently transfected the TetO7. LV B-lymphoblastoid reporter cell line with an mRNA encoding for the full-length DNMT3B (without fusing it to the tetR DNA binding domain; the amino acid sequence of the DNMT3B is in Table 1) with or without the two ATRs. Remarkably, DNMT3B overexpression significantly rescued activity of the tetR:K+tetR:D3A combination, enabling stable eGFP silencing in 52% of the treated cells, with a 65-fold increase compared to tetR:K+tetR:D3A alone (FIG. 19D). Of note, DNMT3B overexpression generated also a 2.9-fold increase in the silencing efficiency of the tetR:D3A condition (FIG. 19E).

Overall, these data clearly show that the un-targeted DNMT3B can effectively rescue activity of the DNMT3A+KRAB combination in refractory cell types.

### Silencing of the BCL11A Gene Using Both CRISPR/dCas9—and TALE-Based ATRs.

We then exploited the ATR combination to silence BCL11A, a gene whose repression has been proposed as a potential therapeutic intervention for β-Thalassemia and Sickle Cell Anaemia. To easily assess activity of the ATRs on the BCL11A gene, we targeted the tdTomato transgene within the third exon of the gene in human B-lymphoblastoid cells by means of gene targeting with CRISPR/Cas-based technology (FIG. 20A). Such targeting strategy allows expressing the tdTomato transgene from the regulatory sequences of the BCL 11A gene, thus faithfully reporting the expression level of this gene. We then enriched to near purity the tdTomato-positive cells by cell sorting, and targeted 4 CpG islands in the promoter/enhancer region of this gene with CRISPR/dCas9-based ATRs containing DNMT3A or DNMT3L. Each of the 4 CpG island was individually interrogated using a separate pool of gRNAs (also known as CRISPR; the nucleotide sequences of the gRNAs are reported in Table 6). By comparing tdTomato expression between treated and untreated controls, we were able to measure the relative contribution of each island to the expression of BCL11A (FIG. 20B). When compared to control-treated cells, silencing of each CpG island was associated with long-term stable repression of BCL11A expression (shown here as % of tdTomato-negative cells), although the extent of silencing of this gene varied according to the CpG island targeted by the ATRs. We then selected the CpG island 31 and 38 for further studies aiming at assessing the activity of the triple ATR combination. In these studies we also included all possible double-ATR combinations and the single KRAB-based ATRs. Remarkably, all conditions tested were able to induce significant levels of gene silencing, with epigenetic editing of CpG 38 (the best responsive island in the previous experiments) with the triple ATR combination resulting in up to 55% gene silencing (FIG. 20C). Finally, we designed 17 different TALE-based ATRs targeting the CpG island 31 and 38 (7 and 10 TALE protein, respectively; the amino acid sequences of these TALEs and their cognate target sequences are listed in Table 7; FIG. 20D top schematics), and tested their silencing activity either as a triple-ATR combination or as KRAB-based ATR. Silencing of both CpG island with all triple ATR combination resulted in effective and long-term silencing of BCL11A (reaching up to 55% of tdTomato-negative cells), while silencing with TALE:KRAB was associated with different degrees of gene silencing, some being as efficient as the triple ATR combination, while others being completely inactive. Overall, these data show the feasibility of permanently silencing the human BCL1A gene.

### Silencing of Additional Human Endogenous Genes Using CRISPR/dCas9-Based ATRs.

We finally challenged our epigenetic silencing technology against two additional human endogenous genes, that are the Interferon (alpha, beta and omega) Receptor 1 (IFNAR1) gene and the Vascular Endothelial Growth Factor A (VEGFA) gene. Both genes show a CpG island at the gene promoter/enhancer region. Therefore, we designed 13 gRNAs against the IFNAR1 CpG island (FIG. 20E, Top) 3 gRNAs against the VEGFA CpG island (FIG. 20F, Top) (the nucleotide sequences of the gRNAs are reported in Table 6). Interestingly, by electroporating K562 cells with plasmids encoding for the pool of 13 gRNAs against the IFNAR1 gene plus the triple dCas9-based ATRs combination, we achieved long-term downregulation of the IFNAR1 transcript level (0.22 fold change) in treated cells compared to the untreated sample (FIG. 20E, Bottom). Furthermore, by electroporating K562 cells with plasmids encoding for the pool of 3 gRNAs against the VEGFA gene plus the triple dCas9-based ATRs combination, we achieved long-term downregulation of the VEGFA transcript level (0.57 fold change) in treated cells compared to the untreated sample (FIG. 20F, Bottom). Overall, these data show the feasibility of silencing various human endogenous genes by CRISPR/dCas9-based ATRs.

### Material and Methods

### Lentiviral Vectors and ATR Constructions

The ATR-reporter Lentiviral Vectors (LV) containing the TetO7 sequence or the TALE binding sites, and the DNMT3B gRNA-expressing LV were generated from the self-inactivating transfer construct pCCLsin.cPPT.hPGK.eGFP.Wpre (Follenzi, A. et al. (2000) Nat. Genet. 25: 217-22), while ATR-expressing Bid.LVs were generated from the transfer construct pCCLsin.cPPT.dLNGFR.mhCMV.hPGK.GFP.Wpre (Gentner, B. et al. (2010) Sci. Transl. Med. 2: 58ra84). The doxycycline-inducible Cas9 expressing vector was obtained from Addgene (pCW-Cas9; #50661; Wang, T. et al. (2014) Science 343: 80-4). LV stocks were prepared as previously described (Follenzi, A. et al. (2002) Methods Mol. Med. 69:259-74). Briefly, HEK293T cells were cotransfected by calcium phosphate precipitation with the transfer construct plasmid, the pMD. Lg/pRRE packaging plasmid, the pMD2.VSV-G envelope-encoding plasmid and pRSV-Rev in the following amounts: 35/12.5/9/6.25 μg DNA per 15 cm dish, respectively. Vector particles were concentrated 300-fold by ultracentrifugation and titred by serial dilution on HEK293T cells as previously described (Cantore, A. et al. (2015) Sci. Transl. Med. 7: 277ra28). All other tetR-based ATRs were generated by replacing the KRAB domain in tetR:KRAB (which is itself discussed in Szulc, J. et al. (2006) Nat. Methods 3: 109-16) with the relevant other effector domains. TALE-based ATRs were generated using a modified version of the Golden Gate TALEN Kit 2.0a (Addgene, Kit #1000000024; Cermak, T. et al. (2011) Nucleic Acids Res. 39: e82) containing the following architectural changes: the Golden Gate TALE C- and N-terminal subregions were replaced with the +163 and a +63 terminal deletions, respectively. These constructs were adapted to accommodate in frame the effector domains. The Cas9-based ATRs were generated by replacing the VP160 transactivator from the plasmid pAC154-dual-dCas9VP160-sgExpression (Addgene #48240; Cheng, A. W. et al. (2013) Cell Res. 23: 1163-71) with the effector domains or with the catalytic domain of TET1.

### Cell Culture Conditions and Engineering

Human Epstein-Barr Virus-immortalised B lymphocytes (B-lymphoblastoid cells) and U-937 cells were maintained in RPMI-1640 (Sigma); HEK293T and K-562 in IMDM (Sigma); NIH/3T3 in DMEM (Sigma). All media were supplemented with 10% FBS (Foetal Bovine Serum; EuroClone), L-glutamine (EuroClone) and 1% Penicillin/Streptomycin (100 U/mL final concentration; EuroClone). Cells were cultured at 37° C. in a 5% CO₂ humidified incubator. The reporter cell lines were generated by transducing the cells with the indicated ATR-reporter LVs at a Multiplicity of Infection (MOI) of 0.1, and then enriched for eGFP expression using a MoFlo XDP Cell Sorter (Beckman Coulter). The reporter cell lines with targeted integration were generated as follows: i) for the insertion of the eGFP-cassette into the AAVS1 locus, we co-transfected a donor construct (containing the TetO7 sequence downstream or upstream of the cassette; 1.5 μg of donor plasmid) and the previously described AAVS1-ZFNs in forms or mRNAs (0.5 μg each ZFN; Lombardo, A. et al. (2011) Nat. Methods 8: 861-9). Single-cell derived clones were then obtained by limiting dilution plating, and analysed by Southern blot to confirm targeted integration of the cassette as previously described (Lombardo, A. et al. (2011) Nat. Methods 8: 861-9); ii) for the insertion of the tdTomato cassette within the third exon of BCL11A, we co-transfected a donor construct containing the tdTomato transgene fused to 2A self-catalytic peptide (2 μg), together with a plasmid encoding for Cas9 (1 μg) and another expressing a gRNA targeting exon 3 (125 ng; sequence of the gRNA: 5′-GGAGCTCTAATCCCCACGCCTGG-3′, SEQ ID NO: 110); iii) a similar targeting strategy to that used for BCL11A was used to insert a splice acceptor-IRES-tdTomato cassette into intron 1 of B2M (sequence of the gRNA: 5′-AGGCTACTAGCCCCATCAAGAGG-3′. SEQ ID NO: 111). Both the tdTomato cell lines were generated by FACS-sorting of the positive cells.

To test activity of the ATRs, the reporter cell lines were transduced with the ATR-expressing Bid.LV at a MOI of 10, or transfected with plasmids or in vitro transcribed mRNAs expressing the ATRs (4D-Nucleofector™ System; Lonza) according to the manufacturer's instruction for K-562, U937 and NIH/3T3, or using the pulse program EW-113 and the SF solution for B-lymphoblastoid cells. We routinely transfected 2 μg of nucleic acid (both plasmid and in vitro transcribed mRNA) for each tetR- or TALE-based ATR, except for experiments conducted in non-saturating conditions in which we used 500 ng of plasmid encoding for each of the ATRs. On the other hand, we electroporated 1-2 μg of plasmid encoding for the dCas9-based ATRs and 125-250 ng of plasmids expressing for the gRNAs. In vitro transcribed mRNAs were produced as previously described (Genovese, P. et al. (2014) Nature 510: 235-40). When indicated, cells were treated with 1 μM of 5-Aza-2-deoxycytidine (AZA, Sigma) or with 12 μg/mL of doxycycline (Sigma). The AZA-containing media was replaced every day, and the cells were analysed by flow cytometry at day 4 and 7 after treatment. When indicated, cells were treated with 500 U/mL of Recombinant Human IFN-γ (R&D Systems). The IFN-γ-containing media was replaced every day, and the cells were analysed by flow cytometry at day 2 and 4 after treatment. Cord-blood derived CD34+ cells from healthy donors were purchased from Lonza. 10⁶ CD34+ cells/mL were stimulated overnight in serum-free StemSpan medium (StemCell Technologies) supplemented with penicillin, streptomycin and the following human early-acting cytokines: Stem Cell Factor (SCF) 50 ng/mL, Flt3 ligand (Flt3-L) 50 ng/mL, thrombopoietin (TPO) 50 ng/mL, and interleukin 6 (IL-6) 50 ng/mL (all purchased from Peprotech). The cells were then transduced with the TetO7-reporter LV at MOI of 30-50. After 48 hours, the cells were electroporated with 2 μg of the ATR-encoding mRNAs (P3 Primary Cell 4D-Nucleofector X Kit, program EO-100; Lonza). 1 μM of SR1 (BioVision Inc.) was added at every medium change. After one week in stimulating media, cells were grown in liquid culture in IMDM 10% FBS. For CFC assays, 800 cells/plate were seeded one day after electroporation in methylcellulose-based medium (MethoCult H4434, StemCell Technologies). Two weeks after plating, colonies were counted and identified according to morphological criteria and analysed by flow cytometry.

Resting T-lymphocytes were isolated from Peripheral Blood Mononuclear Cells (PBMCs) of healthy donors by leukapheresis and Ficoll-Hypaque gradient separation. The cells were activated and sorted using magnetic beads conjugated to antibodies to CD3 and CD28 (ClinExVivo CD3/CD28; Invitrogen), following the manufacturer instructions, and grown at a concentration of 1×10⁶ cells per mL in RPMI (Sigma) supplemented with penicillin, streptomycin, 10% FBS and 5 ng/mL of IL-7 and IL-15 (PeproTech) as previously described (Kaneko, S. et al. (2009) Blood 113: 1006-15). After three days in culture, the cells were transduced with the TetO7-reporter the LV at the MOI of 10. Three days after transduction, the cells were washed and electroplated with 2 μg of mRNA encoding for the ATRs. To test silencing resistance to polyclonal TCR stimulation, we co-cultured the bulk-treated T-lymphocytes with a pool of 6000 rad irradiated PMBCs from unrelated donors and 10000 rad irradiated JY cells in presence of anti-CD3 antibody (OKT3) 30 ng/mL (Orthoclone, Milan, Italy) and human recombinant IL-2 50 U/mL (PrepoTech). Regarding silencing of B2M in primary T-lymphocytes, these cells were isolated from PBMCs of a healthy donor by leukapheresis, Ficoll-Hypaque gradient separation and final selection with the Pan T Cell Isolation Kit (Miltenyi Biotec). The T-cells were then activated with magnetic beads conjugated to antibodies to CD3 and CD28 (ClinExVivo CD3/CD28; Invitrogen), following the manufacturer's instructions, and grown at a concentration of 1×10⁶ cells per mL in RPMI (Sigma) supplemented with penicillin, streptomycin, 10% FBS and 5 ng/mL of IL-7 and IL-15 (PeproTech) as previously described (Kaneko, S. et al. (2009) Blood 113: 1006-15). After three days in culture, the cells were electroporated with in vitro transcribed mRNA encoding for TALE-based ATRs and kept in culture for further 2 weeks, with beads removal 4 days post electroporation. The use of human CB-derived CD34+ cells and of primary T-lymphocytes was approved by the San Raffaele Hospital Bioethical Committee.

### Flow Cytometry and Gene Expression Analyses

For immunophenotypic analysis of Bid.LV transduced cells, CD34+ cells and their progeny, and T lymphocytes (performed by FACSCanto II; BD Pharmingen) we used the following antibodies.

| Antibody | Conjugated | Company |
|---|---|---|
| anti-human | PE | Miltenyi Biotec |
| CD133/2 | | |
| anti-human CD34 | PECy7 | BD Pharmingen |
| anti-human CD90 | APC | BD Pharmingen |
| anti-human CD45 | PB | BioLegend |
| anti-human CD3 | PE | BD Pharmingen |
| anti-human CD13 | APC | BD Pharmingen |
| anti-human CD33 | PeCy7 | BD Pharmingen |
| anti-human CD235a | PE, APC | BD Pharmingen |
| anti-human B2M | PE | Biolegend |
| anti-human MHC-I | APC | Santa Cruz Biotechnology, Inc |
| anti-human CD271 | Alexa Fluor 647 | BD Pharmingen |

Aminoactinomicin D (7-AAD) positive, nonviable cells were excluded from the analysis, and 1-5×10⁵ viable cells were scored per analysis. Single stained and FMO stained cells were used as controls.

For the gene expression analyses, total RNA extracted from 2-6×10⁶ cells (RNeasy Mini kit; Qiagen) was reverse-transcribed using random examers according to the SuperScript III First-Strand Synthesis System (Invitrogen) manufacturer's protocol. We analysed 15-100 ng of cDNA from K-562 and HE K293T cells in triplicate with TaqMan Gene Expression assays (Applied Biosystems).

| Catalog number | Gene Name [ID] |
|---|---|
| Hs00215284_m1 | NLR family, pyrin domain containing 2 [NLRP2] |
| Hs00212574_m1 | Glycoprotein VI (platelet) [GP6] |
| Hs00293416_m1 | Retinol dehydrogenase 13 (all-trans/9-cis) [RDH13] |
| Hs00373719_m1 | EPS8-like 1 [EPS8L1] |
| Hs01085949_m1 | Protein phosphatase 1, regulatory (inhibitor) subunit |
| | 12C [PPP1R12C] |
| Hs00165957_m1 | Troponin T type 1 (skeletal, slow) [TNNT1] |
| Hs00162848_m1 | Troponin I type 3 (cardiac) [TNNI3] |
| Hs00332766_m1 | Chromosome 19 open reading frame 51 [C19orf51] |
| Hs00162516_m1 | Synaptotagmin V [SYT5] |
| Hs00936202_m1 | Protein tyrosine phosphatase, receptor type, |
| | H [PTPRH] |
| Hs00382401_m1 | Transmembrane protein 86B [TMEM86B] |
| Hs00208777_m1 | SAPS domain family, member 1 [SAPS1] |
| Hs99999907_m1 | Beta-2-microglobulin [B2M] |
| Hs02758991_g1 | Glyceraldehyde-3-phosphate dehydrogenase [GAPDH] |
| Hs01060665_g1 | Actin beta [ACTB] |
| Hs99999909_m1 | Hypoxanthine phosphoribosyltransferase 1 [HPRT1] |
| Hs00973637_m1 | 2′-5′-oligoadenylate synthetase 1 [OAS1] |
| Hs00276752_m1 | Spastic paraplegia 11 [SPG11] |
| Hs01388797_m1 | Protein associated with topoisomerase II |
| | homolog 2 [PATL2] |
| Hs04399718_m1 | Tripartite motif containing 69 [TRIM69] |
| Hs00900055_m1 | Vascular Endothelial Growth Factor A [VEGFA] |
| Hs01066116_m1 | Interferon (alpha, beta and omega) Receptor 1 |
| | [IFNAR1] |

The gene expression assay used to detect the eGFP transcript was previously described (Lombardo, A. et al. (2011) Nat. Methods 8: 861-9). Real-time PCRs were performed with a ViiA 7 Real-Time PCR System (Applied Biosystems) and dedicated software was used to extract raw data (Ct and raw fluorescence). Genes with a Ct value≥37 were excluded from the analyses. The relative expression level of each gene was calculated by the ΔΔCt method, normalised to HPRT or B2M expression (housekeeping gene controls), and represented as fold change relative to the mock-treated samples (calibrator).

### Molecular Analyses

For bisulfite sequencing, genomic DNA was extracted with DNeasy Blood & Tissue Kit or QIAamp DNA Mini Kit (QIAGEN) and then treated with EpiTect Bisulfite kit (Qiagen) according to manufacturer's instructions. The converted products were then used to FO R amplify the B2M-promoter region using the primers listed below. FOR fragments were purified and cloned into pCRII-TOPO TA (Invitrogen), and five to ten clones for each sample were verified by sequencing using the M13 universal primer.

| | |
|---|---|
| BIS B2M #1 F | GTTGTGTTTTTTGGGGAAGTTAG |
| | (SEQ ID NO: 112) |
| BIS B2M #1 R | AAAATTCCTCCCTATATCCTTA |
| | (SEQ ID NO: 113) |
| BIS B2M #2 F | AAGAATGGAGAAATTTTGTAGGGAATT |
| | (SEQ ID NO: 114) |
| BIS B2M #2 R | ACCACCAAAAAAAACTTAAAAAAAA |
| | (SEQ ID NO: 115) |
| BIS B2M #3 F | TTTTTTTGGTTTGGAGGTTATTTAG |
| | (SEQ ID NO: 116) |
| BIS B2M #3 R | CAAAACACATAAAATCCTTAACACA |
| | (SEQ ID NO: 117) |
| BIS B2M #4 F | TTTTAGATTGGAGAGTTGTGGATTT |
| | (SEQ ID NO: 118) |
| BIS B2M #4 R | AATTTTACAACTCCCCTAACTAACA |
| | (SEQ ID NO: 119) |

Chromatin immunoprecipitation (ChIP) analysis was performed as previously described (Lombardo, A. et al. (2011) Nat. Methods 8: 861-9) using 5-10 μg of ChIP-grade antibodies (Abcam) raised against the human H3 or the RNA Polymerase II CTD repeat YSPTSPS. IgG isotypes were also used as controls. The primers used for these studies are listed below. The percentage of enrichment of RNA PolII for each investigated site was calculated by the ΔCt method using the Input as normaliser.

| | |
|---|---|
| B2M −241 F | GCAAGTCACTTAGCATCTCTGGG |
| | (SEQ ID NO: 120) |
| B2M −241 R | TTGCTGTCTGTACATCGGCG |
| | (SEQ ID NO: 121) |
| B2M +158 F | TCTCTCGCTCCGTGACTTCC |
| | (SEQ ID NO: 122) |
| B2M +158 R | CGCTTCCCCGAGATCCAGCCC |
| | (SEQ ID NO: 123) |
| B2M +315 F | AGGGGAGACCTTTGGCCTAC |
| | (SEQ ID NO: 124) |
| B2M +315 R | CTCTGACGCTTATCGACGCC |
| | (SEQ ID NO: 125) |
| B2M +580 F | AGACTGGAGAGCTGTGGACTTCG |
| | (SEQ ID NO: 126) |
| B2M +580 R | GCCAAGCATTCTACAAACGTCG |
| | (SEQ ID NO: 127) |
| B2M +1529 F | CAGTCAGGGGAGCTGTAAAACC |
| | (SEQ ID NO: 128) |
| B2M +1529 R | TTGCCAGGTACTTAGAAAGTGC |
| | (SEQ ID NO: 129) |
| B2M +3307 F | CCTTGGGTTGATCCACTTAGG |
| | (SEQ ID NO: 130) |
| B2M +3307 R | TAGTAGAGTGCCTGGGACATAGC |
| | (SEQ ID NO: 131) |
| B2M +3969 F | GTGTCTGGGTTTCATCCATCCG |
| | (SEQ ID NO: 132) |
| B2M +3969 R | GCTGAAAGACAAGTCTGAATGC |
| | (SEQ ID NO: 133) |
| B2M +5082 F | AGGATAAAGGCAGGTGGTTACC |
| | (SEQ ID NO: 134) |
| B2M +5082 R | AGATGTCCAATGTGGAAATGGC |
| | (SEQ ID NO: 135) |
| CCR5 −305 F | AGTCTGACTACAGAGGCCACTGG |
| | (SEQ ID NO: 136) |
| CCR5 R −255 | AGGCAAATGAGACCCCAAACAGC |
| | (SEQ ID NO: 137) |
| PPP1R12C −861 F | TAAGAACCGAGGACAAGTAGTGC |
| | (SEQ ID NO: 138) |
| PPP1R12C −768R | TGCTGGGATGACGAGCGTAAGC |
| | (SEQ ID NO: 139) |

### Statistical Analysis

One-way ANOVA test with Bonferroni's multiple comparison post-test was used to assess statistical significance of differences in gene expression among all samples (P<0.05).

TABLE 1
ZNF10
ALSPQHSAVTQGSIIKNKEGMDAKSLTAWSRTLVTFKDVFVDFTREEWKLLDTAQQIVYRNVMLENYK
NLVSLGYQLTKPDVILRLEKGEEPWLVEREIHQETHPDSETAFEIKSSV (SEQ ID NO: 1)
DNMT3A
TYGLLRRREDWPSRLQMFFANNHDQEFDPPKVYPPVPAEKRKPIRVLSLFDGIATGLLVLKDLGIQVD
RYIASEVCEDSITVGMVRHQGKIMYVGDVRSVTQKHIQEWGPFDLVIGGSPCNDLSIVNPARKGLYEG
TGRLFFEFYRLLHDARPKEGDDRPFFWLFENVVAMGVSDKRDISRFLESNPVMIDAKEVSAAHRARYF
WGNLPGMNRPLASTVNDKLELQECLEHGRIAKFSKVRTITTRSNSIKQGKDQHFPVFMNEKEDILWCT
EMERVFGFPVHYTDVSNMSRLARQRLLGRSWSVPVIRHLFAPLKEYFACV (SEQ ID NO: 8)
EZH2 (short variant)
NVSCKNCSIQRGSKKHLLLAPSDVAGWGIFIKDPVQKNEFISEYCGEIISQDEADRRGKVYDKYMCSF
LFNLNNDFVVDATRKGNKIRFANHSVNPNCYAKVMMVNGDHRIGIFAKRAIQTGEELFFDYRYSQADA
LKYVGIEREMEIP (SEQ ID NO: 140)
EZH2 (long variant)
RLWAAHCRKIQLKKDGSSNHVYNYQPCDHPRQPCDSSCPCVIAQNFCEKFCQCSSECQNRFPGCRCKA
QCNTKQCPCYLAVRECDPDLCLTCGAADHWDSKNVSCKNCSIQRGSKKHLLLAPSDVAGWGIFIKDPV
QKNEFISEYCGEIISQDEADRRGKVYDKYMCSFLFNLNNDFVVDATRKGNKIRFANHSVNPNCYAKVM
MVNGDHRIGIFAKRAIQTGEELFFDYRYSQADALKYVGIEREMEIP (SEQ ID NO: 141)
TLE1
MFPQSRHPTPHQAAGQPFKFTIPESLDRIKEEFQFLQAQYHSLKLECEKLASEKTEMQRHYVMYYEMS
YGLNIEMHKQTEIAKRLNTICAQVIPFLSQEHQQQVAQAVERAKQVTMAELNAIIGQQQLQAQHLSHG
(SEQ ID NO: 142)
G9A (short variant)
LNRKLRLGVGNRAIRTEKIICRDVARGYENVPIPCVNGVDGEPCPEDYKYISENCETSTMNIDRNITH
LQHCTCVDDCSSSNCLCGQLSIRCWYDKDGRLLQEFNKIEPPLIFECNQACSCWRNCKNRVVQSGIKV
RLQLYRTAKMGWGVRALQTIPQGTFICEYVGELISDAEADVREDDSYLFDLDNKDGEVYCIDARYYGN
ISRFINHLCDPNIIPVRVFMLHQDLRFPRIAFFSSRDIRTGEELGFDYGDRFWDIKSKYFTCQCGSEK
CKHSAEAIALEQSRLARLDPHPELLPELGSLPPVNT (SEQ ID NO: 143)
G9A (long variant)
LEKALVIQESERRKKLRFHPRQLYLSVKQGELQKVILMLLDNLDPNFQSDQQSKRTPLHAAAQKGSVE
ICHVLLQAGANINAVDKQQRTPLMEAVVNNHLEVARYMVQRGGCVYSKEEDGSTCLHHAAKIGNLEMV
SLLLSTGQVDVNAQDSGGWTPIIWAAEHKHIEVIRMLLTRGADVTLTDNEENICLHWASFTGSAAIAE
VLLNARCDLHAVNYHGDTPLHIAARESYHDCVLLFLSRGANPELRNKEGDTAWDLTPERSDVWFALQL
NRKLRLGVGNRAIRTEKIICRDVARGYENVPIPCVNGVDGEPCPEDYKYISENCETSTMNIDRNITHL
QHCTCVDDCSSSNCLCGQLSIRCWYDKDGRLLQEFNKIEPPLIFECNQACSCWRNCKNRVVQSGIKVR
LQLYRTAKMGWGVRALQTIPQGTFICEYVGELISDAEADVREDDSYLFDLDNKDGEVYCIDARYYGNI
SRFINHLCDPNIIPVRVFMLHQDLRFPRIAFFSSRDIRTGEELGFDYGDRFWDIKSKYFTCQCGSEKC
KHSAEAIALEQSRLARLDPHPELLPELGSLPPVNT (SEQ ID NO: 144)
SETDB1
MSSLPGCIGLDAATATVESEEIAELQQAVVEELGISMEELRHFIDEELEKMDCVQQRKKQLAELETWV
IQKESEVAHVDQLFDDASRAVTNCESLVKDFYSKLGLQYRDSSSEDESSRPTEIIEIPDEDDDVLSID
SGDAGSRTPKDQKLREAMAALRKSAQDVQKFMDAVNKKSSSQDLHKGTLSQMSGELSKDGDLIVSMRI
LGKKRTKTWHKGTLIAIQTVGPGKKYKVKFDNKGKSLLSGNHIAYDYHPPADKLYVGSRVVAKYKDGN
QVWLYAGIVAETPNVKNKLRFLIFFDDGYASYVTQSELYPICRPLKKTWEDIEDISCRDFIEEYVTAY
PNRPMVLLKSGQLIKTEWEGTWWKSRVEEVDGSLVRILFLDDKRCEWIYRGSTRLEPMFSMKTSSASA
LEKKQGQLRTRPNMGAVRSKGPVVQYTQDLTGTGTQFKPVEPPQPTAPPAPPFPPAPPLSPQAGDSDL
ESQLAQSRKQVAKKSTSFRPGSVGSGHSSPTSPALSENVSGGKPGINQTYRSPLGSTASAPAPSALPA
PPAPPVFHGMLERAPAEPSYRAPMEKLFYLPHVCSYTCLSRVRPMRNEQYRGKNPLLVPLLYDFRRMT
ARRRVNRKMGFHVIYKTPCGLCLRTMQEIERYLFETGCDFLFLEMFCLDPYVLVDRKFQPYKPFYYIL
DITYGKEDVPLSCVNEIDTTPPPQVAYSKERIPGKGVFINTGPEFLVGCDCKDGCRDKSKCACHQLTI
QATACTPGGQINPNSGYQYKRLEECLPTGVYECNKRCKCDPNMCTNRLVQHGLQVRLQLFKTQNKGWG
IRCLDDIAKGSFVCIYAGKILTDDFADKEGLEMGDEYFANLDHIESVENFKEGYESDAPCSSDSSGVD
LKDQEDGNSGTEDPEESNDDSSDDNFCKDEDFSTSSVWRSYATRRQTRGQKENGLSETTSKDSHPPDL
GPPHIPVPPSIPVGGCNPPSSEETPKNKVASWLSCNSVSEGGFADSDSHSSFKTNEGGEGRAGGSRME
AEKASTSGLGIKDEGDIKQAKKEDTDDRNKMSVVTESSRNYGYNPSPVKPEGLRRPPSKTSMHQSRRL
MASAQSNPDDVLTLSSSTESEGESGTSRKPTAGQTSATAVDSDDIQTISSGSEGDDFEDKKNMTGPMK
RQVAVKSTRGFALKSTHGIAIKSTNMASVDKGESAPVRKNTRQFYDGEESCYIIDAKLEGNLGRYLNH
SCSPNLFVQNVFVDTHDLRFPWVAFFASKRIRAGTELTWDYNYEVGSVEGKELLCCCGAIECRGRLL
(SEQ ID NO: 12)
SUV420H2
MGPDRVTARELCENDDLATSLVLDPYLGFRTHKMNVSPVPPLRRQQHLRSALETFLRQRDLEAAYRAL
TLGGWTARYFQSRGPRQEAALKTHVYRYLRAFLPESGFTILPCTRYSMETNGAKIVSTRAWKKNEKLE
LLVGCIAELREADEGLLRAGENDFSIMYSTRKRSAQLWLGPAAFINHDCKPNCKFVPADGNAACVKVL
RDIEPGDEVTCFYGEGFFGEKNEHCECHTCERKGEGAFRTRPREPALPPRPLDKYQLRETKRRLQQGL
DSGSRQG (SEQ ID NO: 145)
HP1-α
MGKKTKRTADSSSSEDEEEYVVEKVLDRRVVKGQVEYLLKWKGFSEEHNTWEPEKNLDCPELISEFMK
KYKKMKEGENNKPREKSESNKRKSNFSNSADDIKSKKKREQSNDIARGFERGLEPEKIIGATDSCGDL
MFLMKWKDTDEADLVLAKEANVKCPQIVIAFYEERLTWHAYPEDAENKEKETAKS
(SEQ ID NO: 146)
DNMT3L
MAAIPALDPEAEPSMDVILVGSSELSSSVSPGTGRDLIAYEVKANQRNIEDICICCGSLQVHTQHPLF
EGGICAPCKDKFLDALFLYDDDGYQSYCSICCSGETLLICGNPDCTRCYCFECVDSLVGPGTSGKVHA
MSNWVCYLCLPSSRSGLLQRRRKWRSQLKAFYDRESENPLEMFETVPVWRRQPVRVLSLFEDIKKELT
SLGFLESGSDPGQLKHVVDVTDTVRKDVEEWGPFDLVYGATPPLGHTCDRPPSWYLFQFHRLLQYARP
KPGSPRPFFWMFVDNLVLNKEDLDVASRFLEMEPVTIPDVHGGSLQNAVRVWSNIPAIRSRHWALVSE
EELSLLAQNKQSSKLAAKWPTKLVKNCFLPLREYFKYFSTELTSSL (SEQ ID NO: 11)
DNMT3B
MVAELISEEDLEFMKGDTRHLNGEEDAGGREDSILVNGACSDQSSDSPPILEAIRTPEIRGRRSSSRL
SKREVSSLLSYTQDLTGDGDGEDGDGSDTPVMPKLFRETRTRSESPAVRTRNNNSVSSRERHRPSPRS
TRGRQGRNHVDESPVEFPATRSLRRRATASAGTPWPSPPSSYLTIDLTDDTEDTHGTPQSSSTPYARL
AQDSQQGGMESPQVEADSGDGDSSEYQDGKEFGIGDLVWGKIKGFSWWPAMVVSWKATSKRQAMSGMR
WVQWFGDGKFSEVSADKLVALGLFSQHFNLATFNKLVSYRKAMYHALEKARVRAGKTFPSSPGDSLED
QLKPMLEWAHGGFKPTGIEGLKPNNTQPENKTRRRTADDSATSDYCPAPKRLKTNCYNNGKDRGDEDQ
SREQMASDVANNKSSLEDGCLSCGRKNPVSFHPLFEGGLCQTCRDRFLELFYMYDDDGYQSYCTVCCE
GRELLLCSNTSCCRCFCVECLEVLVGTGTAAEAKLQEPWSCYMCLPQRCHGVLRRRKDWNVRLQAFFT
SDTGLEYEAPKLYPAIPAARRRPIRVLSLFDGIATGYLVLKELGIKVGKYVASEVCEESIAVGTVKHE
GNIKYVNDVRNITKKNIEEWGPFDLVIGGSPCNDLSNVNPARKGLYEGTGRLFFEFYHLLNYSRPKEG
DDRPFFWMFENVVAMKVGDKRDISRFLECNPVMIDAIKVSAAHRARYFWGNLPGMNRPVIASKNDKLE
LQDCLEYNRIAKLKKVQTITTKSNSIKOGKNQLFPVVMNGKEDVLWCTELERIFGFPVHYTDVSNMGR
GARQKLLGRSWSVPVIRHLFAPLKDYFACE (SEQ ID NO: 36)

TABLE 2
TALE forward
MGKPIPNPLLGLDSTGGMAPKKKRKVDGGVDLRTLGYSQOQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPEQVVAIASNIGGKQALETVQRLLPVLCQAHGL
TPEQVVAIASHDGGKQALETVQRLLPVLCQAHGLTPEQVVAIASHDGGKQ
ALETVQRLLPVLCQAHGLTPEQVVAIASHDGGKQALETVQRLLPVLCQAH
GLTPEQVVAIASNIGGKQALETVQRLLPVLCQAHGLTPEQVVAIASNNGG
KQALETVQRLLPVLCQAHGLTPEQVVAIASNIGGKQALETVQRLLPVLCQ
AHGLTPEQVVAIASNGGGKQALETVQRLLPVLCQAHGLTPEQVVAIASNG
GGKQALETVQRLLPVLCQAHGLTPEQVVAIASNNGGKQALETVQRLLPVL
CQAHGLTPEQVVAIASNNGGKQALETVQRLLPVLCQAHGLTPEQVVAIAS
HDGGKQALETVQRLLPVLCQAHGLTPEQVVAIASHDGGKQALETVQRLLP
VLCQAHGLTPEQVVAIASHDGGKQALETVQRLLPVLCQAHGLTPEQVVAI
ASHDGGKQALETVQRLLPVLCQAHGLTPEQVVAIASNIGGKQALETVQRL
LPVLCQAHGLTPEQVVAIASHDGGKQALETVQRLLPVLCQAHGLTPQQVV
AIASNGGGRPALESIVAQLSRPDPALAALTNDHLVALACLGGRPALDAVK
KGLPHAPALIKRTNRRIPERTSHRVA (SEQ ID NO: 14)
TALE reverse
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPEQVVAIASNIGGKQALETVQRLLPVLCQAHGL
TPEQVVAIASHDGGKQALETVQRLLPVLCQAHGLTPEQVVAIASHDGGKQ
ALETVQRLLPVLCQAHGLTPEQVVAIASNGGGKQALETVQRLLPVLCQAH
GLTPEQVVAIASNIGGKQALETVQRLLPVLCQAHGLTPEQVVAIASNNGG
KQALETVQRLLPVLCQAHGLTPEQVVAIASNIGGKQALETVQRLLPVLCQ
AHGLTPEQVVAIASNNGGKQALETVQRLLPVLCQAHGLTPEQVVAIASNN
GGKQALETVQRLLPVLCQAHGLTPEQVVAIASNIGGKQALETVQRLLPVL
CQAHGLTPEQVVAIASNNGGKQALETVQRLLPVLCQAHGLTPEQVVAIAS
NIGGKQALETVQRLLPVLCQAHGLTPEQVVAIASNIGGKQALETVQRLLP
VLCQAHGLTPEQVVAIASNIGGKQALETVQRLLPVLCQAHGLTPEQVVAI
ASNNGGKQALETVQRLLPVLCQAHGLTPEQVVAIASNNGGKQALETVQRL
LPVLCQAHGLTPEQVVAIASNGGGKQALETVQRLLPVLCQAHGLTPQQVV
AIASNGGGRPALESIVAQLSRPDPALAALTNDHLVALACLGGRPALDAVK
KGLPHAPALIKRTNRRIPERTSHRVA (SEQ ID NO: 15)
Nucleotide sequences of the corresponding
TALE binding sites
TALE forward
5′-TACCCAGATTGGCCCCACT-3′ (SEQ ID NO: 34)
TALE reverse
5′-TACCTAGAGGAGAAAGGTT-3′ (SEQ ID NO: 35)

TABLE 3
Amino acid sequences of the TALEs
targeting the B2M promoter region
TALE #1
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASHDGGKQALETVQRLLPVLCQDHGL
TPDQVVAIASNGGGKQALETVQRLLPVLCQDHGLTPDQVVAIASHDGGKQ
ALETVQRLLPVLCQDHGLTPDQVVAIASNGGGKQALETVQRLLPVLCQDH
GLTPDQVVAIASHDGGKQALETVQRLLPVLCQDHGLTPDQVVAIASHDGG
KQALETVQRLLPVLCQDHGLTPDQVVAIASNGGGKQALETVQRLLPVLCQ
DHGLTPDQVVAIASNIGGKQALETVQRLLPVLCQDHGLTPDQVVAIASHD
GGKQALETVQRLLPVLCQDHGLTPDQVVAIASHDGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASNHGGKQALETVQRL
LPVLCQDHGLTPDQVVAIASHDGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG (SEQ ID NO: 16)
TALE #2
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASNNGGKQALETVQRLLPVLCQDHGL
TPDQVVAIASNNGGKQALETVQRLLPVLCQDHGLTPDQVVAIASNGGGKQ
ALETVQRLLPVLCQDHGLTPDQVVAIASHDGGKQALETVQRLLPVLCQDH
GLTPDQVVAIASHDGGKQALETVQRLLPVLCQDHGLTPDQVVAIASNGGG
KQALETVQRLLPVLCQDHGLTPDQVVAIASNGGGKQALETVQRLLPVLCQ
DHGLTPDQVVAIASHDGGKQALETVQRLLPVLCQDHGLTPDQVVAIASHD
GGKQALETVQRLLPVLCQDHGLTPDQVVAIASNGGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASHDGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG (SEQ ID NO: 18)
TALE #3
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASHDGGKQALETVQRLLPVLCQDHGL
TPDQVVAIASNNGGKQALETVQRLLPVLCQDHGLTPDQVVAIASHDGGKQ
ALETVQRLLPVLCQDHGLTPDQVVAIASNGGGKQALETVQRLLPVLCQDH
GLTPDQVVAIASHDGGKQALETVQRLLPVLCQDHGLTPDQVVAIASHDGG
KQALETVQRLLPVLCQDHGLTPDQVVAIASNNGGKQALETVQRLLPVLCQ
DHGLTPDQVVAIASNGGGKQALETVQRLLPVLCQDHGLTPDQVVAIASNN
GGKQALETVQRLLPVLCQDHGLTPDQVVAIASNIGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASNGGGKQALETVQRLLP
VLCQDHGLTPDQVVAIASHDGGKQALETVQRLLPVLCQDHGLTPDQVVAI
ASHDGGKQALETVQRLLPVLCQDHGLTPDQVVAIASHDGGKQALETVQRL
LPVLCQDHGLTPDQVVAIASNGGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG (SEQ ID NO: 20)
Nucleotide sequences of the corresponding
TALE binding sites
TALE #1
5′-TCTCTCCTACCCTCCCGCT-3′ (SEQ ID NO: 17)
TALE #2
5′-TGGTCCTTCCTCTCCCGCT-3′ (SEQ ID NO: 19)
TALE #3
5′-TCGCTCCGTGACTTCCCTT-3′ (SEQ ID NO: 21)

TABLE 4
Catalytically inactive Cas9 (dCas9)
MGGRRVRWEVYISRALWLTREPTAYWLIEINTTHYRETQATGATMYPYDV
PDYASPKKKRKVEASDKKYSIGLAIGTNSVGWAVITDEYKVPSKKFKVLG
NTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEE
GIKELGSQILKEHPVENTQLQNEKLYLYYLQNGRDMYVDQELDINRLSDY
DVDAIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILD
SRMNTKYDENDKLIREVKVITLKSKLVSDFRKDFQFYKVREINNYHHAHD
AYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFL
YLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRY
TSTKEVLDATLIHQSITGLYETRIDLSQLGGDSPKKKRKVG
(SEQ ID NO: 22)
Nucleotide sequences of the target sites of
the B2M gRNAs
gRNA #1
TATAAGTGGAGGCGTCGCGC (SEQ ID NO: 23)
gRNA #2
GCCCGAATGCTGTCAGCTTC (SEQ ID NO: 24)
gRNA #3
TGCGTCGCTGGCTTGGAGAC (SEQ ID NO: 25)
gRNA #4
CCAATCAGGACAAGGCCCGC (SEQ ID NO: 26)
gRNA #5
AGGGTAGGAGAGACTCACGC (SEQ ID NO: 27)
gRNA #6
GCGGGCCACCAAGGAGAACT (SEQ ID NO: 28)
gRNA #7
GCTACTCTCTCTTTCTGGCC (SEQ ID NO: 29)
gRNA #8
CTCCCGCTCTGCACCCTCTG (SEQ ID NO: 30)
gRNA #9
TTTGGCCTACGGCGACGGGA (SEQ ID NO: 31)
gRNA #10
GGGGCAAGTAGCGCGCGTCC (SEQ ID NO: 32)
gRNA #11
TAGTCCAGGGCTGGATCTCG (SEQ ID NO: 33)
Nucleotide sequences of the B2M gRNAs

| gRNA #1: | UAUAAGUGGAGGCGUCGCGC (SEQ ID NO: 149) |
|---|---|
| gRNA #2: | GCCCGAAUGCUGUCAGCUUC (SEQ ID NO: 150) |
| gRNA #3: | UGCGUCGCUGGCUUGGAGAC (SEQ ID NO: 151) |
| gRNA #4: | CCAAUCAGGACAAGGCCCGC (SEQ ID NO: 152) |
| gRNA #5: | AGGGUAGGAGAGACUCACGC (SEQ ID NO: 153) |
| gRNA #6: | GCGGGCCACCAAGGAGAACU (SEQ ID NO: 154) |
| gRNA #7: | GCUACUCUCUCUUUCUGGCC (SEQ ID NO: 155) |
| gRNA #8: | CUCCCGCUCUGCACCCUCUG (SEQ ID NO: 156) |
| gRNA #9: | UUUGGCCUACGGCGACGGGA (SEQ ID NO: 157) |
| gRNA | GGGGCAAGUAGCGCGCGUCC (SEQ ID NO: 158) |
| #10: | |
| gRNA | UAGUCCAGGGCUGGAUCUCG (SEQ ID NO: 159) |
| #11: | |

TABLE 5
Amino acid sequence of the TALE A targeting
the B2M promoter region
TALE A
MGKPIPNPLLGLDSTGGMAPKKKRKVDGGVDLRTLGYSQQQQEKIKPKVR
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLNLTPDQVVAIASNNGGKQALETVQRLLPVLCQDHGL
TPDQVVAIASHDGGKQALETVQRLLPVLCQDHGLTPDOVVAIASNGGGKQ
ALETVQRLLPVLCQDHGLTPDQVVAIASHDGGKQALETVQRLLPVLCQDH
GLTPDQVVAIASNNGGKQALETVQRLLPVLCQDHGLTPDQVVAIASHDGG
KQALETVQRLLPVLCQDHGLTPDQVVAIASNNGGKQALETVQRLLPVLCQ
DHGLTPDQVVAIASHDGGKQALETVQRLLPVLCQDHGLTPDQVVAIASNG
GGKQALETVQRLLPVLCQDHGLTPDQVVAIASNIGGKQALETVQRLLPVL
CQDHGLTPDQVVAIASHDGGKQALETVQRLLPVLCQDHGLTPDQVVAIAS
NGGGKQALETVQRLLPVLCQDHGLTPDQVVAIASHDGGKQALETVQRLLP
VLCQDHGLTPDQVVAIASNGGGKQALETVQRLLPVLCQDHGLTPDQVVAI
ASHDGGKQALETVQRLLPVLCQDHGLTPDQVVAIASNGGGKQALETVQRL
LPVLCQDHGLTPDQVVAIASHDGGKQALETVQRLLPVLCQDHGLTPDQVV
AIASNGGGKQALESIVAQLSRPDPALAALTNDHLVALACLGGRPALDAVK
KGLPHAPALIKRTNRRIPERTSHRVAGSGGG (SEQ ID NO: 147)
Nucleotide sequence of the corresponding
TALE binding site
TALE A
5′-TGCTCGCGCTACTCTCTCT-3′ (SEQ ID NO: 148)

TABLE 6
Nucleotide sequences of the gRNAs
targeting BCL11A

| | |
|---|---|
| gRNA #1 against CpG 105: | GCCUUUCUGCAGACGUUCCC |
| | (SEQ ID NO: 71) |
| gRNA #2 against CpG 105: | UGGGUGUGCGCCUUGGCCGG |
| | (SEQ ID NO: 72) |
| gRNA #3 against CpG 105: | CGGUGGUGAGAUGACCGCCU |
| | (SEQ ID NO: 73) |
| gRNA #4 against CpG 105: | GGAAUGUGCUCACGGCGCCG |
| | (SEQ ID NO: 74) |
| gRNA #5 against CpG 105: | GACUGCCCGCGCUUUGUCCU |
| | (SEQ ID NO: 75) |
| gRNA #6 against CpG 105: | CCAGAGUCUGGCCCCCGGAG |
| | (SEQ ID NO: 76) |
| gRNA #7 against CpG 105: | UCUGCGACCCUUAGGAGCCG |
| | (SEQ ID NO: 77) |
| gRNA #8 against CpG 105: | GAGCGCCCCGCCAAGCGACU |
| | (SEQ ID NO: 78) |
| gRNA #9 against CpG 105: | CAAGUCUCCAGGAGCCCGCG |
| | (SEQ ID NO: 79) |
| gRNA #10 against CpG 105: | CGCGGAAUCCAGCCUAAGUU |
| | (SEQ ID NO: 80) |
| gRNA #11 against CpG 105: | CCCGCUGCGGAGCUGUAACU |
| | (SEQ ID NO: 81) |
| gRNA #1 against CpG 31: | CGCUCCUGAGUCCGCGGAGU |
| | (SEQ ID NO: 82) |
| gRNA #2 against CpG 31: | CACGGCUCUCCCCGUCGCCG |
| | (SEQ ID NO: 83) |
| gRNA #3 against CpG 31: | CCGCCUUUUGUUCCGGCCAG |
| | (SEQ ID NO: 84) |
| gRNA #4 against CpG 31: | GCGCGAGGAGCCGGCACAAA |
| | (SEQ ID NO: 85) |
| gRNA #5 against CpG 31: | GCCACUUUCUCACUAUUGUG |
| | (SEQ ID NO: 86) |
| gRNA #6 against CpG 31: | GCUGCCUCUGAGGUUCGGUC |
| | (SEQ ID NO: 87) |
| gRNA #7 against CpG 31: | AAGGGCAGGAGCUAGGGCCG |
| | (SEQ ID NO: 88) |
| gRNA #8 against CpG 31: | GAGCCCGGACUGCUGCCUCC |
| | (SEQ ID NO: 89) |
| gRNA #1 against CpG 38: | GUUUACAAGCACCGCGUGUG |
| | (SEQ ID NO: 90) |
| gRNA #2 against CpG 38: | AACAGACAGAGGACCGAGCG |
| | (SEQ ID NO: 91) |
| gRNA #3 against CpG 38: | GGCGCCGGGUGGGCGAUCCG |
| | (SEQ ID NO: 92) |
| gRNA #4 against CpG 38: | GGUCGGGCAAGGCCCGGGCG |
| | (SEQ ID NO: 93) |
| gRNA #5 against CpG 38: | AAGAGGUCUCGGCAUUGUGC |
| | (SEQ ID NO: 94) |
| gRNA #6 against CpG 38: | GUUCCACAGCUUCGGGACCG |
| | (SEQ ID NO: 95) |
| gRNA #7 against CpG 38: | GAAAUCGGCUGGGUGAAACU |
| | (SEQ ID NO: 96) |
| gRNA #8 against CpG 38: | GCAGUGUCUCCGCGCCAGCC |
| | (SEQ ID NO: 97) |
| gRNA #9 against CpG 38: | CCUCCCCUCCCCUCCGCCCU |
| | (SEQ ID NO: 98) |
| gRNA #1 against CpG 115: | UCCUCCUGUCCCGGGGUUAA |
| | (SEQ ID NO: 99) |
| gRNA #2 against CpG 115: | CAUCUUUUGGGACACUCUAGG |
| | (SEQ ID NO: 100) |
| gRNA #3 against CpG 115: | AAGUCAGGCCCUUCUUCGGAA |
| | (SEQ ID NO: 101) |
| gRNA #4 against CpG 115: | GCAGCCUGGACUGCGCGCCC |
| | (SEQ ID NO: 102) |
| gRNA #5 against CpG 115: | UGCCCGGCGAUUCUCGUCCG |
| | (SEQ ID NO: 103) |
| gRNA #6 against CpG 115: | UGAGCCAUUCGGUCGCUAGG |
| | (SEQ ID NO: 104) |
| gRNA #7 against CpG 115: | GGUGGUACUGAGGACCGGGA |
| | (SEQ ID NO: 105) |
| gRNA #8 against CpG 115: | AUUUUCUGGGUGCUCAGAGG |
| | (SEQ ID NO: 107) |
| gRNA #9 against CpG 115: | UGGUCUCAGCUCGCGCACGG |
| | (SEQ ID NO: 108) |
| gRNA #10 against CpG 115: | ACAAAGACAUACGGGGUGAU |
| | (SEQ ID NO: 109) |

Nucleotide sequences of the gRNAs
targeting IFNAR1

| | |
|---|---|
| gRNA #1: | AGGAACGGCGCGUGCGCGGA |
| | (SEQ ID NO: 106) |
| gRNA #2: | AAGAGGCGGCGCGUGCGUAG |
| | (SEQ ID NO: 161) |
| gRNA #3: | GGGCGGUGUGACUUAGGACG |
| | (SEQ ID NO: 162) |
| gRNA #4: | CCAGAUGAUGGUCGUCCUCC |
| | (SEQ ID NO: 163) |
| gRNA #5: | GACCCUAGUGCUCGUCGCCG |
| | (SEQ ID NO: 164) |
| gRNA #6: | UGGGUGUUGUCCGCAGCCGC |
| | (SEQ ID NO: 165) |
| gRNA #7: | ACGGGGGCGGCGAUGCUGUU |
| | (SEQ ID NO: 166) |
| gRNA #8: | GACCGAAGGUUUCCCAGACU |
| | (SEQ ID NO: 167) |
| gRNA #9: | GUCGGGUUUAAUCUUUGGCG |
| | (SEQ ID NO: 168) |
| gRNA #10: | CGCUCCCGAGGACCCGUACA |
| | (SEQ ID NO: 169) |
| gRNA #11: | CGGGUCCCACCCCCGUGAAA |
| | (SEQ ID NO: 170) |
| gRNA #12: | UCAAACUCGACACAAAGCUC |
| | (SEQ ID NO: 171) |
| gRNA #13: | GCGGAGCCGCGGUACUUUCC |
| | (SEQ ID NO: 172) |

Nucleotide sequences of the gRNAs
targeting VEGFA

| | |
|---|---|
| gRNA #1: | GGCGCGCGCGCUAGGUGGGA |
| | (SEQ ID NO: 173) |
| gRNA #2: | AGAGAGGCUCACCGCCCACG |
| | (SEQ ID NO: 174) |
| gRNA #3: | GUACGUGCGGUGACUCCGGU |
| | (SEQ ID NO: 175) |

TABLE 7
Amino acid sequence of the TALEs targeting the BCL11A gene
TALE BCL11A #1
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASNNGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNNGGKQALETVQRLLPVLCODHGLTPDQVVAIASHDGGKQALETVQRLLPVLCQDHGLTPDQ
VVAIASNIGGKQALESIVAQLSRPDPALAALTNDHLVALACLGGRPALDAVKKGLPHAPALIKRTNRR
IPERTSHRVAGSGGG (SEQ ID NO: 37)
Nucleotide sequence of the corresponding TALE binding site
5′-TCCAAAAGCCAGTCTCACC-3′ (SEQ ID NO: 38)
TALE BCL11A #2
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNNGGKQALETVQRLLPVLCQDHGLTPDQVVAIASNIGGKQALETVQRLLPVLCQDHGLTPDQ
VVAIASNNGGKQALETVQRLLPVLCQDHGLTPDQVVAIASNIGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASNIGGKQALESIVAQLSRPDPALAALTNDHLVALACLGGRPALDAVKKGLPHAPALIKRTNRR
IPERTSHRVAGSGGG (SEQ ID NO: 39)
5′-TCTCCCCGGGAATCGTTTT-3′ (SEQ ID NO: 40)
TALE BCL11A #3
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASNNGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNHGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 41)
5′-TCCTCCCGCTGCACACTTG-3′ (SEQ ID NO: 42)
TALE BCL11A #4
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASNNGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASNIGGKQALETVQRLLPVLCQDHGLTPDQVVAIASNNGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASHDGGKQALESIVAQLSRPDPALAALTNDHLVALACLGGRPALDAVKKGLPHAPALIKRTNRR
IPERTSHRVAGSGGG (SEQ ID NO: 43)
5′-TAGTCATCCCCACAATAGT-3′ (SEQ ID NO: 44)
TALE BCL11A #5
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASNIGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 45)
5′-TCCCGCTGCCTTTTGTGCC-3′ (SEQ ID NO: 46)
TALE BCL11A #6
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCODHGLTPDQ
VVAIASNNGGKQALETVQRLLPVLCQDHGLTPDQVVAIASNNGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 47)
5′-TCCTCGCGCTTGCCCTCCC-3′ (SEQ ID NO: 48)
TALE BCL11A #7
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASNHGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASNNGGKQALESIVAQLSRPDPALAALTNDHLVALACLGGRPALDAVKKGLPHAPALIKRTNRR
IPERTSHRVAGSGGG (SEQ ID NO: 49)
5′-TCCCCCGGCCCTAGCTCCT-3′ (SEQ ID NO: 50)
TALE BCL11A #8
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNNGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNHGGKQALETVQRLLPVLCQDHGLTPDQ
VVAIASHDGGKQALETVQRLLPVLCQDHGLTPDQVVAIASNHGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 51)
5′-TCCTGGTCCGCCCCCAGCA-3′ (SEQ ID NO: 52)
TALE BCL11A #9
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 53)
5′-TGCCGAGACCTCTTCTCGA-3′ (SEQ ID NO: 54)
TALE BCL11A #10
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASNNGGKQALETVKRLLPVLCQDHGLTPDQ
VVAIASNNGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 55)
5′-TCGGCTTTGCAAAGCATTT-3′ (SEQ ID NO: 56)
TALE BCL11A #11
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASNNGGKQALETVQRLLPVLCQDHGLTPDQVVAIASNNGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 57)
5′-TGCAAAGCCGAGTTTCACC-3′ (SEQ ID NO: 58)
TALE BCL11A #12
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNHGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASNNGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASHDGGKQALESIVAQLSRPDPALAALTNDHLVALACLGGRPALDAVKKGLPHAPALIKRTNRR
IPERTSHRVAGSGGG (SEQ ID NO: 59)
5′-TACAGTTGCCCTGCAAAAT-3′ (SEQ ID NO: 60)
TALE BCL11A #13
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNIGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNNGGKQALESIVAQLSRPDPALAALTNDHLVALACLGGRPALDAVKKGLPHAPALIKRTNRR
IPERTSHRVAGSGGG (SEQ ID NO: 61)
5′-TCCGCCCTGGGTACTTTCT-3′ (SEQ ID NO: 62)
TALE BCL11A #14
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASNIGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASHDGGKQALETVQRLLPVLCQDHGLTPDOVVAIASNIGGKQALETVQRLLPVLCQDHGLTPDQ
VVAIASNGGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASHDGGKQALETVQRLLPVLCQDHGLTPDQVVAIASNIGGKQALETVQRLLPVLCQDHGLTPDQ
VVAIASNIGGKQALETVQRLLPVLCQDHGLTPDQVVAIASNGGGKQALETVQRLLPVLCQDHGLTPDQ
VVAIASNIGGKQALETVQRLLPVLCQDHGLTPDQVVAIASNNGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 63)
5′-TCTCTTGTCCACAGCTCGG-3′ (SEQ ID NO: 64)
TALE BCL11A #15
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASNGGGKQALETVQRLLPVLCQDHGLTPDQ
VVAIASNNGGKQALETVQRLLPVLCQDHGLTPDQVVAIASHDGGKQALETVQRLLPVLCQDHGLTPDQ
VVAIASHDGGKQALESIVAQLSRPDPALAALTNDHLVALACLGGRPALDAVKKGLPHAPALIKRTNRR
IPERTSHRVAGSGGG (SEQ ID NO: 65)
5′-TCTCCCGCTGACTGCGCCT-3′ (SEQ ID NO: 66)
TALE BCL11A #16
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNGGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALESIVAQLSRPDPALAALTNDHLVALACLGGRPALDAVKKGLPHAPALIKRTNRR
IPERTSHRVAGSGGG (SEQ ID NO: 67)
5′-TCCCTTGCTGCCAAACTTT-3′ (SEQ ID NO: 68)
TALE BCL11A #17
MGKPIPNPLLGLDSTGGMAPKKKRKVDGGVDLRTLGYSQQQQEKIKPKVRSTVAQHHEALVGHGFTHA
HIVALSQHPAALGTVAVKYQDMIAALPEATHEAIVGVGKQWSGARALEALLTVAGELRGPPLQLDTGQ
LLKIAKRGGVTAVEAVHAWRNALTGAPLNLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNNGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASNIGGKQALETVQRLLPVLCQDHGLTPDQVVAIASNNGGKQALETVQRLLPVLCQDHGLTPDQ
VVAIASHDGGKQALETVQRLLPVLCQDHGLTPDQVVAIASNGGGKQALETVQRLLPVLCQDHGLTPDQ
VVAIASHDGGKQALETVQRLLPVLCQDHGLTPDQVVAIASHDGGKQALETVQRLLPVLCQDHGLTPDQ
VVAIASNGGGKQALESIVAQLSRPDPALAALTNDHLVALACLGGRPALDAVKKGLPHAPALIKRTNRR
IPERTSHRVAGSGGG (SEQ ID NO: 69)
5′-TGGGCCCTCACGCCTTTCT-3′ (SEQ ID NO: 70)

All publications mentioned in the above specification are herein incorporated by reference. Various modifications and variations of the described products, uses, methods and kits of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1-44. (canceled)

45. An artificial transcription repressor (ATR) comprising a DNA-binding domain operably linked to two or more domains selected from groups (a), (b), or (c); wherein the two or more domains comprise: (a) and (b), or (a) and (c); wherein:
(a) is a Krüppel-associated box (KRAB) domain or homologue thereof;
(b) is a DNA methyltransferase 3A (DNMT3A), DNA methyltransferase 3B (DNMT3B) or DNA methyltransferase 1 (DNMT1) domain or homologue thereof; and
(c) is a DNA (cytosine-5)-methyltransferase 3-like (DNMT3L) domain or homologue thereof;
optionally wherein the DNA-binding domain comprises a zinc finger domain or a Clustered Regularly Interspaced Short Palindromic Repeats (CRISPR)/Cas system.

46. The ATR of claim 45, wherein the DNA-binding domain comprises a zinc finger domain.

47. The ATR of claim 45, wherein the DNA-binding domain comprises a CRISPR/Cas system.

48. The ATR of claim 47, wherein the CRISPR/Cas system comprises Cas9.

49. The ATR of claim 48, wherein the Cas9 is derived from S. pyogenes.

50. The ATR of claim 48, wherein the Cas9 is catalytically active.

51. The ATR of claim 48, wherein the Cas9 is catalytically dead.

52. A polynucleotide encoding the ATR of claim 47.

53. A polynucleotide encoding the ATR of claim 48.

54. A polynucleotide encoding the ATR of claim 49.

55. A polynucleotide encoding the ATR of claim 50.

56. A polynucleotide encoding the ATR of claim 51.

57. A method of silencing a target gene comprising the step of administering the ATR of claim 45 to a cell.

58. A method of gene therapy comprising administering the ATR of claim 45 to a subject.

59. A method of silencing a target gene comprising the step of administering the polynucleotide of claim 52 to a cell.

60. A method of gene therapy comprising administering the polynucleotide of claim 52 to a subject.

61. A cell comprising the ATR of claim 45.

62. A cell wherein said cell is a descendant of the cell of claim 61.

63. The ATR of claim 47, wherein the ATR comprises a DNA-binding domain operably linked to a KRAB domain or homologue thereof, a DNTM3A domain or a homologue thereof, and a DNMT3L domain or homologue thereof.

64. The ATR of claim 47, wherein the ATR comprises a DNA-binding domain operably linked to a KRAB domain or homologue thereof, and a DNMT3L domain or homologue thereof.
